(19) United States
(12) Patent Application Publication
(10) Pub. No.: US 2011/0183954 A1
(43) Pub. Date: Jul. 28, 2011

(54) TRICYCLIC 2,4-DIAMINO-L,3,5-TRIAZINE DERIVATIVES USEFUL FOR THE TREATMENT OF CANCER AND MYELOPROLIFERATIVE DISORDERS

(75) Inventors: Lynsie Almeida, Waltham, MA (US); Claudio Edmundo Chuaqui, Waltham, MA (US); Stephanos Loannidis, Waltham, MA (US); Bo Peng, Waltham, MA (US); Mei Su, Waltham, MA (US)

(73) Assignee: ASTRAZENECA AB, Sodertalje (SE)

(21) Appl. No.: 12/997,054

(22) PCT Filed: Dec. 17, 2009
(86) PCT No.: PCT/GB09/50655

§ 371 Date: Mar. 9, 2011

(30) Foreign Application Priority Data
Jun. 11, 2008 (US) 61060784

(51) Int. Cl. A61K 31/397; C07D 413/14; A61K 31/5377; C07D 401/14; A61K 31/53; A61P 35/00

(52) U.S. Cl. 514/210.2; 544/113; 514/236.2; 544/216; 514/245

(57) ABSTRACT

The present invention relates to compounds of Formula (I): (I) and to their salts, pharmaceutical compositions, methods of use, and methods for their preparation. These compounds provide a treatment for myeloproliferative disorders and cancer.

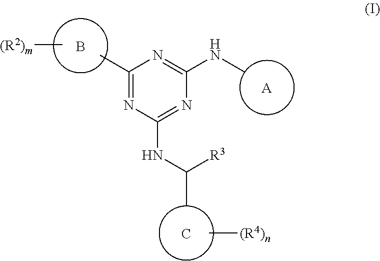

### FIELD OF THE INVENTION

The present invention relates to novel compounds, their pharmaceutical compositions, methods for producing them, and their methods of use. In addition, the present invention relates to therapeutic methods for the treatment and prevention of cancers and to the use of this compound in the manufacture of medicaments for use in the treatment and prevention of myeloproliferative disorders and cancers.

### BACKGROUND OF THE INVENTION

The JAK (Janus-associated kinase)/STAT (signal transducers and activators of transcription) signalling pathway is involved in a variety of hyperproliferative and cancer related processes including cell-cycle progression, apoptosis, angiogenesis, invasion, metastasis and evasion of the immune system (Haura et al., Nature Clinical Practice Oncology, 2005, 2(6), 315-324; Verna et al., Cancer and Metastasis Reviews, 2003, 22, 423-434).

The JAK family consists of four non-receptor tyrosine kinases Tyk2, JAK1, JAK2, and JAK3, which play a critical role in cytokine- and growth factor mediated signal transduction. Cytokine and/or growth factor binding to cell-surface receptor(s), promotes receptor dimerization and facilitates activation of receptor-associated JAK by autophosphorylation. Activated JAK phosphorylates the receptor, creating docking sites for SH2 domain-containing signalling proteins, in particular the STAT family of proteins (STAT1, 2, 3, 4, 5a, 5b and 6). Receptor-bound STATs are themselves phosphorylated by JAKs, promoting their dissociation from the receptor, and subsequent dimerization and translocation to the nucleus. Once in the nucleus, the STATs bind DNA and cooperate with other transcription factors to regulate expression of a number of genes including, but not limited to, genes encoding apoptosis inhibitors (e.g. Bcl-XL, Mcl-1) and cell cycle regulators (e.g. Cyclin D1/D2, c-myc) (Haura et al., Nature Clinical Practice Oncology, 2005, 2(6), 315-324; Verna et al., Cancer and Metastasis Reviews, 2003, 22, 423-434).

Over the past decade, a considerable amount of scientific literature linking constitutive JAK and/or STAT signalling with hyperproliferative disorders and cancer has been published. Constitutive activation of the STAT family, in particular STAT3 and STATS, has been detected in a wide range of cancers and hyperproliferative disorders (Haura et al., Nature Clinical Practice Oncology, 2005, 2(6), 315-324). Furthermore, aberrant activation of the JAK/STAT pathway provides an important proliferative and/or anti-apoptotic drive downstream of many kinases (e.g. Flt3, EGFR) whose constitutive activation have been implicated as key drivers in a variety of cancers and hyperproliferative disorders (Tibes et al., Annu Rev Pharmacol Toxicol 2550, 45, 357-384; Choudhary et al., International Journal of Hematology 2005, 82(2), 93-99; Sordella et al., Science 2004, 305, 1163-1167). In addition, impairment of negative regulatory proteins, such as the suppressors of cytokine signalling (SOCS) proteins, can also influence the activation status of the JAK/STAT signalling pathway in disease (J C Tan and Rabkin R, Pediatric Nephrology 2005, 20, 567-575).

Several mutated forms of JAK2 have been identified in a variety of disease settings. For example, translocations resulting in the fusion of the JAK2 kinase domain with an oligomerization domain, TEL-JAK2, Bcr-JAK2 and PCM1-JAK2, have been implicated in the pathogenesis of various hematologic malignancies (S D Turner and Alesander D R, Leukemia, 2006, 20, 572-582). More recently, a unique acquired mutation encoding a valine-to-phenylalanine (V617F) substitution in JAK2 was detected in a significant number of polycythemia vera, essential thrombocythemia and idiopathic myelofibrosis patients and to a lesser extent in several other diseases. The mutant JAK2 protein is able to activate downstream signalling in the absence of cytokine stimulation, resulting in autonomous growth and/or hypersensitivity to cytokines and is believed to play a critical role in driving these diseases (M J Percy and McMullin M F, Hematological Oncology 2005, 23(3-4), 91-93).

JAKs (in particular JAK3) play an important biological roles in the immunosuppressive field and there are reports of using JAK kinase inhibitors as tools to prevent organ transplant rejections (Changelian, P. S. et al, Science, 2003, 302, 875-878). Merck (Thompson, J. E. et al Bioorg. Med. Chem. Lett. 2002, 12, 1219-1223) and Incyte (WO2005/105814) reported imidazole based JAK2/3 inhibitors with enzyme potency at single nM levels. Publications including Vertex PCT publications have described azaindoles as JAK inhibitors (WO2005/95400).

### SUMMARY OF THE INVENTION

In accordance with the present invention, the applicants have hereby discovered novel compounds of Formula (I):

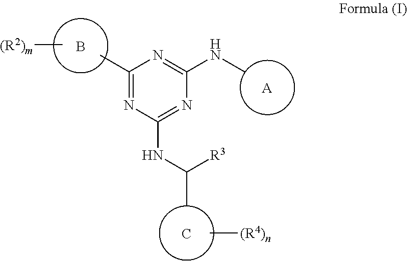

and pharmaceutically acceptable salts thereof.

It is believed that the compounds of Formula (I), or pharmaceutically acceptable salts thereof, possess beneficial efficacious, metabolic, and/or pharmacodynamic properties.

The compounds of Formula (I), or pharmaceutically acceptable salts thereof, are believed to possess JAK kinase inhibitory activity and are accordingly useful for their anti-proliferation and/or pro-apoptotic activity and in methods of treatment of the human or animal body. The invention also relates to processes for the manufacture of said compounds, or pharmaceutically acceptable salts thereof, to pharmaceutical compositions containing them and to their use in the manufacture of medicaments for use in the production of an anti-proliferation and/or pro-apoptotic effect in warm-blooded animals such as man. Also in accordance with the present invention the applicants provide methods of using said compounds, or pharmaceutically acceptable salts thereof, in the treatment of myeloproliferative disorders, myelodysplastic syndrome, and cancer.

The properties of the compounds of Formula (I), or pharmaceutically acceptable salts thereof, are expected to be of value in the treatment of myeloproliferative disorders, myelodysplastic syndrome, and cancer by inhibiting the tyrosine kinases, particularly the JAK family and more particularly JAK1 and JAK2. Methods of treatment target tyrosine kinase activity, particularly the JAK family activity and more particularly JAK2 activity, which is involved in a variety of myeloproliferative disorders, myelodysplastic syndrome and cancer related processes. Thus, inhibitors of tyrosine kinases, particularly the JAK family and more particularly JAK2, are expected to be active against myeloproliferative disorders such as chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and neoplastic disease such as carcinoma of the breast, ovary, lung, colon, prostate or other tissues, as well as leukemias, myelomas and lymphomas, tumors of the central and peripheral nervous system, and other tumor types such as melanoma, fibrosarcoma and osteosarcoma. Tyrosine kinase inhibitors, particularly the JAK family inhibitors and more particularly JAK1 and JAK2 inhibitors are also expected to be useful for the treatment other proliferative diseases including but not limited to autoimmune, inflammatory, neurological, and cardiovascular diseases.

Furthermore, the compounds of Formula (I), or pharmaceutically acceptable salts thereof, are expected to be of value in the treatment or prophylaxis of against myeloproliferative disorders selected from chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and cancers selected from oesophageal cancer, myeloma, hepatocellular, pancreatic, cervical cancer, Ewings sarcoma, neuroblastoma, Kaposi's sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, mesothelioma, renal cancer, lymphoma and leukaemia; particularly myeloma, leukemia, ovarian cancer, breast cancer and prostate cancer.

### DETAILED DESCRIPTION OF THE INVENTION

The present invention relates to compounds of Formula (I):

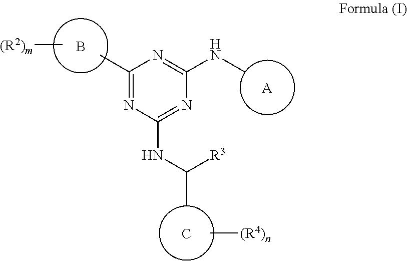

and pharmaceutically acceptable salts thereof, wherein:
Ring A is selected from:

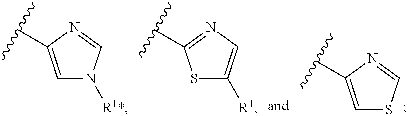

Ring B is 4- to 8-membered saturated heterocyclyl;
Ring C is selected from phenyl and 6-membered heteroaryl;
R¹ is selected from H, halo, —CN, C₁₋₆alkyl, C₂₋₆alkenyl, C₂₋₆alkynyl, carbocyclyl, heterocyclyl, —OR^{1a}, —SR^{1a}, —N(R^{1a})₂, —N(R^{1a})C(O)R^{1b}, —N(R^{1a})N(R^{1a})₂, —NO₂, —N(R^{1a})OR^{1a}, —ON(R^{1a})₂, —C(O)H, —C(O)R^{1b}, —C(O)₂R^{1a}, —C(O)N(R^{1a})₂, —C(O)N(R^{1a})(OR^{1a}), —OC(O)N(R^{1a})₂, —N(R^{1a})C(O)₂R^{1a}, —N(R^{1a})C(O)N(R^{1a})₂, —OC(O)R^{1b}, —S(O)R^{1b}, —S(O)₂R^{1b}, —S(O)₂, —N(R^{1a})₂, —N(R^{1a})S(O)₂R^{1b}, —C(R^{1a})═N(R^{1a}), and —C(R^{1a})═N(OR^{1a}), wherein said C₁₋₆alkyl, C₂₋₆alkenyl, C₂₋₆alkynyl, carbocyclyl, and heterocyclyl are optionally substituted on carbon with one or more R¹⁰, and wherein any —NH— moiety of said heterocyclyl is optionally substituted with R¹⁰*;
R^{1a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
R^{1b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R¹⁰, and wherein any —NH— moiety of said heterocyclyl is optionally substituted with R¹⁰*;
R^{1c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
R¹* is selected from H, —CNC₁₋₆alkyl, carbocyclyl, heterocyclyl, —OR^{1a}, —C(O)H, —C(O)R^{1b}, —C(O)₂R^{1c}, —C(O)N(R^{1a})₂, —S(O)R^{1b}, —S(O)₂R^{1b}, —S(O)₂N(R^{1a})₂, —C(R^{10a})═N(R^{1a}), and —C(R^{1a})═N(OR^{1a}), wherein said C₁₋₆alkyl, carbocyclyl, and heterocyclyl are optionally substituted on carbon with one or more R¹⁰, and wherein any —NH— moiety of said heterocyclyl is optionally substituted with R¹⁰*;
R² in each occurrence is independently selected from halo, —CN, C₁₋₆alkyl, C₂₋₆alkenyl, C₂₋₆alkynyl, carbocyclyl, heterocyclyl, —OR^{2a}, —SR^{2a}, —N(R^{2a})₂, —N(R^{2a})C(O)R^{2b}, —N(R^{2a})N(R^{2a})₂, —NO₂, —N(R^{2a})OR^{2a}, —ON(R^{2a})₂, —C(O)H, —C(O)R^{2b}, —C(O)₂R^{2a}, —C(O)N(R^{2a})₂, —C(O)N(R^{2a})(OR^{2a})—OC(O)N(R^{2a})₂, —N(R^{2a})C(O)₂R^{2a}, —N(R^{2a})C(O)N(R^{2a})₂, —OC(O)R^{2b}, —S(O)R^{2b}, —S(O)₂R^{2b}, —S(O)₂N(R^{2a})₂, —N(R^{2a})S(O)₂R^{2b}, —C(R^{2a})═N(R^{2a}), and —C(R^{2a})═N(OR^{2a}), wherein said C₁₋₆alkyl, C₂₋₆alkenyl, C₂₋₆alkynyl, carbocyclyl, and heterocyclyl in each occurrence are independently and optionally substituted on carbon with one or more R²⁰, and wherein any —NH— moiety of said heterocyclyl is optionally substituted with R²⁰*;
R^{2a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R²⁰, and wherein any —NH— moiety of said heterocyclyl is optionally substituted with R²⁰*;
R^{2b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R²⁰, and wherein any —NH— moiety of said heterocyclyl is optionally substituted with R²⁰*;
R³ is selected from H, halo, —CN, C₁₋₆alkyl, C₂₋₆alkenyl, C₂₋₆alkynyl, carbocyclyl, heterocyclyl, —OR^{3a}, —SR^{3a}, —N(R^{3a})₂, —N(R^{3a})C(O)R^{3b}, —N(R^{3a})N(R^{3a})₂, —NO₂, —N(R^{3a})—OR^{3a}, —O—N(R^{3a})₂, —C(O)H, —C(O)R^{3b}, —C(O)₂R^{3a}, —C(O)N(R^{3a})₂, —C(O)N(R^{3a})(OR^{3a}), —OC(O)N(R^{3a})₂, —N(R^{3a})C(O)₂R³, —N(R^{3a})C(O)N(R^{3a})₂, —OC(O)R^{3b}, —S(O)R^{3b}, —S(O)₂R^{3b}, —S(O)₂N(R^{3a})₂, —N(R^{3a})S(O)₂R^{3b}, —C(R^{3a})═N(R^{3a}), and —C(R^{3a})═N(OR^{3a}), wherein said C₁₋₆alkyl, C₂₋₆alkenyl, C₂₋₆alkynyl, carbocyclyl, and heterocyclyl are optionally substituted on carbon with one or more R³⁰, and wherein any —NH— moiety of said heterocyclyl is optionally substituted with R³⁰*;
R^{1a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R³⁰, and wherein any —NH— moiety of said heterocyclyl is optionally substituted with R³⁰*;
R^{3b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R³⁰, and wherein any —NH— moiety of said heterocyclyl is optionally substituted with R³⁰*;
R⁴ in each occurrence is independently selected from halo, —CN, C₁₋₆alkyl, C₂₋₆alkenyl, C₂₋₆alkynyl, carbocyclyl, heterocyclyl, —OR^{4a}, —SR^{4a}, —N(R^{4a})₂, —N(R^{4a})C(O)R^{4b}, —N(R^{4a})N(R^{4a})₂, —NO₂, —N(R^{4a})—OR^{4a}, —O—N(R^{4a})₂, —C(O)H, —C(O)R^{4b}, —C(O)₂R^{4a}, —C(O)N(R^{4a})₂, —C(O)N(R^{4a})(OR^{4a})—OC(O)N(R^{4a})₂, —N(R^{4a})C(O)₂R^{4a}, —N(R^{4a})C(O)N(R^{4a})₂, —OC(O)R^{4b}, —S(O)R^{4b}, —S(O)₂R^{4b}, —S(O)₂N(R^{4a})₂, —N(R^{4a})S(O)₂R^{4b}, —C(R^{4a})═N(R^{4a}), and —C(R^{4a})═N(OR^{4a}), wherein said C₁₋₆alkyl, C₂₋₆alkenyl, C₂₋₆alkynyl, carbocyclyl, and heterocyclyl in each occurrence are optionally and independently substituted on carbon with one or more R⁴⁰, and wherein any —NH— moiety of said heterocyclyl is optionally substituted with R⁴⁰*;
R^{4a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁴⁰, and wherein any —NH— moiety of said heterocyclyl is optionally substituted with R⁴⁰*;
R^{4b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁴⁰, and wherein any —NH— moiety of said heterocyclyl is optionally substituted with R⁴⁰*;
R¹⁰ in each occurrence is independently selected from halo, —CN, C₁₋₆alkyl, C₂₋₆alkenyl, C₂₋₆alkynyl, carbocyclyl, heterocyclyl, —OR^{10a}, —SR^{10a}, —N(R^{10a})₂, —N(R^{10a})C(O)R^{10b}, —N(R^{10a})N(R^{10a})₂, —NO₂, —N(R^{10a})—OR^{10a}, —O—N(R^{10a})₂, —C(O)H, —C(O)R^{10b}, —C(O)₂R^{10a}, —C(O)N(R^{10a})₂, —C(O)N(R^{10a})(OR^{10a}), —OC(O)N(R^{10a})₂, —N(R^{10a})C(O)₂R^{10a}, —N(R^{10a})C(O)N(R^{10a})₂, —OC(O)R^{10b}, —S(O)R^{10b}, —S(O)₂R^{10b}, —S(O)₂N(R^{10a})₂, —N(R^{10a})S(O)₂R^{10b}, —C(R^{10a})═N(R^{10a}), and —C(R^{10a})═N(OR^{10a}) wherein said C₁₋₆alkyl, C₂₋₆alkenyl, C₂₋₆alkynyl, carbocyclyl, and heterocyclyl in each occurrence are optionally and independently substituted on carbon with one or more R^{a}, and wherein any —NH— moiety of said heterocyclyl is optionally substituted with R^{a}*;
R¹⁰* in each occurrence is independently selected from C₁₋₆alkyl, carbocyclyl, heterocyclyl, —C(O)H, —C(O)R^{10b}, —C(O)₂R^{10c}, —C(O)N(R^{10a})₂, —S(O)R^{10b}, —S(O)₂R^{10b}, —S(O)₂N(R^{10a})₂, —C(R^{10a})═N(R^{10a}), and —C(R^{10a})═N(OR^{10a}) wherein said C₁₋₆alkyl, carbocyclyl, and heterocyclyl in each occurrence are optionally and independently substituted on carbon with one or more R^{a}, and wherein any —NH— moiety of said heterocyclyl is optionally substituted with R^{a}*;
R^{10a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{a}, and wherein any —NH— moiety of said heterocyclyl is optionally substituted with R^{a}*;
R^{10b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{a}, and wherein any —NH— moiety of said heterocyclyl is optionally substituted with R^{a}*;
R^{10c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{a}, and wherein any —NH— moiety of said heterocyclyl is optionally substituted with R^{a}*;
R²⁰ in each occurrence is independently selected from halo, —CN, C₁₋₆alkyl, C₂₋₆alkenyl, C₂₋₆alkynyl, carbocyclyl, heterocyclyl, —OR^{20a}, —SR^{20a}, —N(R^{20a})₂, —N(R^{20a})C(O)R^{20b}, —N(R^{20a})N(R^{20a})₂, —NO₂, —N(R^{20a})—OR^{20a}, —O—N(R^{20a})₂, —C(O)H, —C(O)R^{20b}, —C(O)₂R^{20a}, —C(O)N(R^{20a})₂, —C(O)N(R^{20a})(OR^{20a}), —OC(O)N(R^{20a})₂, —N(R^{20a})C(O)₂R^{20a}, —N(R^{20a})C(O)N(R^{20a})₂, —OC(O)R^{20b}, —S(O)R^{20b}, —S(O)₂R^{20b}, —S(O)₂N(R^{20a})₂, —N(R^{20a})S(O)₂R^{20b}, —C(R^{20a})═N(R^{20a}), and —C(R^{20a})═N(OR^{20a}), wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein any —NH— moiety of said heterocyclyl is optionally substituted with R^{b}*;
R²⁰* in each occurrence is independently selected from C₁₋₆alkyl, carbocyclyl, heterocyclyl, —C(O)H, —C(O)R^{20b}, —C(O)₂R^{20c}, —C(O)N(R^{20a})₂, —S(O)R^{20b}, —S(O)₂R^{20b}, —S(O)₂N(R^{20a})₂, —C(R^{20a})═N(R^{20a}) and —C(R^{20a})═N(OR^{20a}), wherein said C₁₋₆alkyl, carbocyclyl, and heterocyclyl in each occurrence are optionally and independently substituted on carbon with one or more R^{b}, and wherein any —NH— moiety of said heterocyclyl is optionally substituted with R^{b}*;
R^{20a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{b}, and wherein any —NH— moiety of said heterocyclyl is optionally substituted with R^{b}*;
R^{20b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein any —NH— moiety of said heterocyclyl is optionally substituted with R^{b}*;

R^{20c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{b}, and wherein any —NH— moiety of said heterocyclyl is optionally substituted with R^{b}*;

R³⁰ in each occurrence is independently selected from halo, —CN, C₁₋₆alkyl, C₂₋₆alkenyl, C₂₋₆alkynyl, carbocyclyl, heterocyclyl, —OR^{30a}, —SR^{30a}, —N(R^{30a})₂, —N(R^{30a})C(O)R^{30b}, —N(R^{30a})N(R^{30a})₂, —NO₂, —N(R^{30a})—OR^{30a}, —O—N(R^{30a})₂, —C(O)H, —C(O)R^{30b}, —C(O)₂R^{30a}, —C(O)N(R^{30a})₂, —C(O)N(R^{30a})(OR^{30a}), —OC(O)N(R^{30a})₂, —N(R^{30a})C(O)₂R^{30a}, —N(R^{30a})C(O)N(R^{30a})₂, —OC(O)R^{30b}, —S(O)R^{30b}, —S(O)₂R^{30b}, —S(O)₂N(R^{30a})₂, —N(R^{30a})S(O)₂R^{30b}, —C(R^{30a})═N(R^{30a}), and —C(R^{30a})═N(OR^{30a}), wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein any —NH— moiety of said heterocyclyl is optionally substituted with R^{c}*;
R³⁰* in each occurrence is independently selected from C₁₋₆alkyl, carbocyclyl, heterocyclyl, —C(O)H, —C(O)R^{30b}, —C(O)₂R^{30c}, —C(O)N(R^{30a})₂, —S(O)R^{30b}, —S(O)₂R^{30b}, —S(O)₂N(R^{30a})₂, —C(R^{30a})═N(R^{30a}), and —C(R^{30a})═N(OR^{30a}), wherein said C₁₋₆alkyl, carbocyclyl, and heterocyclyl in each occurrence are optionally and independently substituted on carbon with one or more R^{c}, and wherein any —NH— moiety of said heterocyclyl is optionally substituted with R^{c}*;
R^{30a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{c}, and wherein any —NH— moiety of said heterocyclyl is optionally substituted with R^{c}*;
R^{30b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein any —NH— moiety of said heterocyclyl is optionally substituted with R^{c}*;
R^{30c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{c}, and wherein any —NH— moiety of said heterocyclyl is optionally substituted with R^{c}*;
R⁴⁰ in each occurrence is independently selected from halo, —CN, C₁₋₆alkyl, C₂₋₆alkenyl, C₂₋₆alkynyl, carbocyclyl, heterocyclyl, —OR^{40a}, —SR^{40a}, —N(R^{40a})₂, —N(R^{40a})C(O)R^{40b}, —N(R^{40a})N(R^{40a})₂, —NO₂, —N(R^{40a})—OR^{40a}, —O—N(R^{40a})₂, —C(O)H, —C(O)R^{40b}, —C(O)₂R^{40a}, —C(O)N(R^{40a})₂, —C(O)N(R^{40a})(OR^{40a}), —OC(O)N(R^{40a})₂, —N(R^{40a})C(O)₂R^{40a}, —N(R^{40a})C(O)N(R^{40a})₂, —OC(O)R^{40b}, —S(O)R^{40b}, —S(O)₂R^{40b}, —S(O)₂N(R^{40a})₂, —N(R^{40a})S(O)₂R^{40b}, —C(R^{40a})═N(R^{40a}), and —C(R^{40a})═N(OR^{40a}), wherein said C₁₋₆alkyl, C₂₋₆alkenyl, C₂₋₆alkynyl, carbocyclyl, and heterocyclyl in each occurrence are optionally and independently substituted on carbon with one or more R^{d}, and wherein any —NH— moiety of said heterocyclyl is optionally substituted with R^{d}*;
R⁴⁰* in each occurrence is independently selected from C₁₋₆alkyl, carbocyclyl, heterocyclyl, —C(O)H, —C(O)R^{40b}, —C(O)₂R^{40c}—C(O)N(R^{40a})₂, —S(O)R^{40b}, —S(O)₂R^{40b}, —S(O)₂N(R^{40a})₂, —C(R^{40a})═N(R^{40a}), and —C(R^{40a})═N(OR^{40a}), wherein said C₁₋₆alkyl, carbocyclyl, and heterocyclyl in each occurrence are optionally and independently substituted on carbon with one or more R^{d}, and wherein any —NH— moiety of said heterocyclyl is optionally substituted with R^{d}*;
R^{40a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{d}, and wherein any —NH— moiety of said heterocyclyl is optionally substituted with R^{d}*;
R^{40b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{d}, and wherein any —NH— moiety of said heterocyclyl is optionally substituted with R^{d}*;
R^{40c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{d}, and wherein any —NH— moiety of said heterocyclyl is optionally substituted with R^{d}*;
R^{a}, R^{b}, R^{c}, and R^{d} in each occurrence are independently selected from halo, —CN, C₁₋₆alkyl, C₂₋₆alkenyl, C₂₋₆alkynyl, carbocyclyl, heterocyclyl, —OR^{m}, —SR^{m}, —N(R^{m})₂, —N(R^{m})C(O)Rⁿ, —N(R^{m})N(R^{m})₂, —NO₂, —N(R^{m})—OR^{m}, —O—N(R^{m})₂, —C(O)H, —C(O)Rⁿ, —C(O)₂R^{m}, —C(O)N(R^{m})₂, —C(O)N(R^{m})(OR^{m}), —OC(O)N(R^{m})₂, —N(R^{m})C(O)₂R^{m}, —N(R^{m})C(O)N(R^{m})₂, —OC(O)Rⁿ, —S(O)Rⁿ, —S(O)₂Rⁿ, —S(O)₂N(R^{m})₂, —N(R^{m})S(O)₂Rⁿ, —C(R^{m})═N(R^{m}), and —C(R^{m})═N(OR^{m});
R^{a}*, R^{b}*, R^{c}*, and R^{d}* in each occurrence are independently selected from C₁₋₆alkyl, carbocyclyl, heterocyclyl, —C(O)H, —C(O)Rⁿ, —C(O)₂R^{o}, —C(O)N(R^{m})₂, —S(O)Rⁿ, —S(O)₂Rⁿ, —S(O)₂N(R^{m})₂, —C(R^{m})═N(R^{m}), and —C(R^{m})═N(OR^{m});
R^{m} in each occurrence is independently selected from H, C₁₋₆alkyl, carbocyclyl, and heterocyclyl;
Rⁿ in each occurrence is independently selected from C₁₋₆alkyl, C₂₋₆alkenyl, C₂₋₆alkynyl, carbocyclyl, and heterocyclyl;
R^{o} in each occurrence is independently selected from C₁₋₆alkyl, carbocyclyl, and heterocyclyl; and
m is selected from 0, 1, 2, 3, 4, 5, and 6; and
n is selected from 1, 2, 3, and 4.

In this specification the prefix C_{x-y} as used in terms such as C_{x-y}alkyl and the like (where x and y are integers) indicates the numerical range of carbon atoms that are present in the group; for example, C₁₋₄alkyl includes C₁alkyl (methyl), C₂alkyl (ethyl), C₃alkyl (propyl and isopropyl), C₁alkyl (butyl, 1-methylpropyl, 2-methylpropyl, and t-butyl), and C₁₋₃alkyl.

Alkyl—As used herein the term “alkyl” refers to both straight and branched chain saturated hydrocarbon radicals having the specified number of carbon atoms. References to individual alkyl groups such as “propyl” are specific for the straight chain version only and references to individual branched chain alkyl groups such as ‘isopropyl’ are specific for the branched chain version only. In one aspect, “C₁₋₆alkyl” may be C₁₋₃alkyl. In another aspect, “C₁₋₆alkyl” may be methyl.

Alkenyl—As used herein, the term “alkenyl” refers to both straight and branched chain hydrocarbon radicals having the specified number of carbon atoms and containing at least one carbon-carbon double bond. For example, “C₂₋₆alkenyl” includes groups such as C₂₋₆alkenyl, C₂₋₄alkenyl, ethenyl, 2-propenyl, 2-methyl-2-propenyl, 3-butenyl, 4-pentenyl, and 5-hexenyl.

Alkynyl—As used herein, the term “alkynyl” refers to both straight and branched chain hydrocarbon radicals having the specified number of carbon atoms and containing at least one carbon-carbon triple bond. For example, “C₂₋₆alkynyl” includes groups such as C₂₋₆alkynyl, C₂₋₄alkynyl, ethynyl, 2-propynyl, 2-methyl-2-propynyl, 3-butynyl, 4-pentynyl, and 5-hexynyl.

Carbocyclyl—As used herein, the term “carbocyclyl” refers to a saturated, partially saturated, or unsaturated, mono or bicyclic carbon ring that contains 3 to 12 ring atoms, of which one or more —CH₂— groups may be optionally replaced with a corresponding number of —C(O)— groups. Illustrative examples of “carbocyclyl” include, but are not limited to, adamantyl, cyclopropyl, cyclobutyl, cyclopentyl, cyclopentenyl, cyclohexyl, cyclohexenyl, indanyl, naphthyl, oxocyclopentyl, 1-oxoindanyl, phenyl, and tetralinyl. In one aspect, “carbocyclyl” may be cyclopropyl. In another aspect, “carbocyclyl” may be phenyl.

3- to 6-Membered Carbocyclyl—In one aspect, “carbocyclyl” may be “3- to 6-membered carbocyclyl.” The term “3- to 6-membered carbocyclyl” refers to a saturated, partially saturated, or unsaturated monocyclic carbon ring containing 3 to 6 ring atoms, of which one or more —CH₂— groups may be optionally replaced with a corresponding number of —C(O)— groups. Illustrative examples of “3- to 6-membered carbocyclyl” include, but are not limited to, cyclopropyl, cyclobutyl, cyclopentyl, oxocyclopentyl, cyclopentenyl, cyclohexyl, and phenyl. In one aspect, “carboclyl” may be cyclopropyl. In another aspect, cyclopropyl may be phenyl.

Halo—As used herein, the term “halo” refers to fluoro, chloro, bromo and iodo. In one aspect, the term “halo” may refer to fluoro, chloro, and bromo. In another aspect, the term “halo” may refer to fluoro and chloro. In still another aspect, the term “halo” may refer to fluoro.

Heterocyclyl—As used herein, the term “heterocyclyl” refers to a saturated, partially saturated, or unsaturated, mono or bicyclic ring containing 4 to 12 ring atoms of which at least one ring atom is selected from nitrogen, sulfur, and oxygen, and which may, unless otherwise specified, be carbon or nitrogen linked, and of which a —CH₂— group can optionally be replaced by a —C(O)—. Ring sulfur atoms may be optionally oxidized to form S-oxides. Ring nitrogen atoms may be optionally oxidized to form N-oxides. Illustrative examples of the term “heterocyclyl” include, but are not limited to, azetidinyl, 1,1-dioxidothiomorpholinyl, 1,3-benzodioxolyl, 3,5-dioxopiperidinyl, furanyl, imidazolyl, indolyl, isoquinolinyl, isothiazolyl, isoxazolyl, morpholinyl, 2-oxa-5-azabicyclo[2.2.1]hept-5-yl, oxazolyl, oxetanyl, oxopiperazinyl, 2-oxopyrrolidinyl, oxo-1,3-thiazolidinyl, piperazinyl, piperidyl, 2H-pyranyl, pyrazolyl, pyridinyl, pyrrolyl, pyrrolidinyl, pyrimidinyl, pyrazinyl, pyridazinyl, 4-pyridonyl, quinolyl, tetrahydrofuranyl, tetrahydropyranyl, thiazolyl, thiadiazolyl, thiazolidinyl, thiomorpholinyl, thiophenyl, pyridine-N-oxidyl and quinoline-N-oxidyl.

4- to 6-Membered Heterocyclyl—In one aspect, “heterocycl” may be “4- to 6-membered heterocyclyl.” The term “4- to 6-membered heterocyclyl” refers to a saturated, partially saturated, or unsaturated, monocyclic ring containing 4 to 6 ring atoms, of which at least one ring atom is selected from nitrogen, sulfur, and oxygen, and of which a —CH₂— group may be optionally replaced by a —C(O)— group. Unless otherwise specified, “4- to 6-membered heterocyclyl” groups may be carbon or nitrogen linked. Ring nitrogen atoms may be optionally oxidized to form an N-oxide. Ring sulfur atoms may be optionally oxidized to form S-oxides. Illustrative examples of “4- to 6-membered heterocyclyl” include, but are not limited to, azetidin-1-yl, dioxidotetrahydrothiophenyl, 2,4-dioxoimidazolidinyl, 3,5-dioxopiperidinyl, furanyl, imidazolyl, isothiazolyl, isoxazolyl, morpholinyl, oxazolyl, oxetanyl, oxoimidazolidinyl, 3-oxo-1-piperazinyl, 2-oxopyrrolidinyl, 2-oxotetrahydrofuranyl, oxo-1,3-thiazolidinyl, piperazinyl, piperidyl, 2H-pyranyl, pyrazolyl, pyridinyl, pyrrolyl, pyrrolidinyl, pyrimidinyl, pyrazinyl, pyrazolyl, pyridazinyl, 4-pyridonyl, tetrahydrofuranyl, tetrahydropyranyl, thiazolyl, 1,3,4-thiadiazolyl, thiazolidinyl, thiomorpholinyl, thiophenyl, 4H-1,2,4-triazolyl, and pyridine-N-oxidyl.

6-Membered Heteroaryl—In one aspect, “heterocyclyl” and “4- to 6-membered heterocyclyl” may be “6-membered heteroaryl.” The term “6-membered heteroaryl” is intended to refer to a monocyclic, aromatic heterocyclyl ring containing 6 ring atoms. Unless otherwise specified, “6-membered heteroaryl” groups may be carbon or nitrogen linked. Ring nitrogen atoms may be optionally oxidized to form an N-oxide. Ring sulfur atoms may be optionally oxidized to form S-oxides. Illustrative examples of the term “6-membered heteroaryl” include, but are not limited to, pyrazinyl, pyridazinyl, pyrimidinyl, and pyridinyl.

4- to 8-Membered Saturated Heterocyclyl—In one aspect, “heterocyclyl” may be “4- to 8-membered saturated heterocyclyl.” The term “4 to 8-membered saturated heterocyclyl” is intended to refer to a monocyclic or bicyclic saturated ring containing 4 to 8 ring atoms of which at least one ring atom is selected from nitrogen, sulfur, and oxygen, and which may, unless otherwise specified, be carbon or nitrogen linked, and of which a —CH₂— group can optionally be replaced by a —C(O)—. Ring sulfur atoms may be optionally oxidized to form S-oxides. Ring nitrogen atoms may be optionally oxidized to form N-oxides. Illustrative examples of the term “heterocyclyl” include, but are not limited to, azetidinyl, 1,1-dioxidothiomorpholinyl, morpholinyl, 2-oxa-5-azabicyclo[2.2.1]hept-5-yl, oxetanyl, oxopiperazinyl, 2-oxopyrrolidinyl, oxo-1,3-thiazolidinyl, piperazinyl, piperidyl, pyrrolidinyl, tetrahydrofuranyl, tetrahydropyranyl, thiazolidinyl, and thiomorpholinyl.

4- to 6-Membered Saturated Heterocyclyl—In one aspect, “heterocyclyl” and “4- to 8-membered saturated heterocyclyl” may be “4 to 6-membered saturated heterocyclyl.” The term “4- to 6-membered saturated heterocyclyl” refers to a saturated, monocyclic ring containing 4 to 6 ring atoms, of which at least one ring atom is selected from nitrogen, sulfur, and oxygen, and of which a —CH₂— group may be optionally replaced by a —C(O)— group. Unless otherwise specified, “4- to 6-membered saturated heterocyclyl” groups may be carbon or nitrogen linked. Ring nitrogen atoms may be optionally oxidized to form an N-oxide. Ring sulfur atoms may be optionally oxidized to form S-oxides. Illustrative examples of “4- to 6-membered saturated heterocyclyl” include, but are not limited to, azetidinyl, 1,1-dioxidothiomorpholinyl, morpholinyl, oxetanyl, oxopiperazinyl, 2-oxopyrrolidinyl, oxo-1,3-thiazolidinyl, piperazinyl, piperidyl, pyrrolidinyl, tetrahydrofuranyl, tetrahydropyranyl, thiazolidinyl, and thiomorpholinyl.

6-Membered Saturated Heterocyclyl—In one aspect, “heterocyclyl,” “4- to 8-membered saturated heterocyclyl,” and “4 to 6-membered saturated heterocyclyl” may be “6-membered saturated heterocyclyl.” The term “6-membered saturated heterocyclyl” refers to a saturated, monocyclic ring containing 6 ring atoms, of which at least one ring atom is selected from nitrogen, sulfur, and oxygen, and of which a —CH₂— group may be optionally replaced by a —C(O)— group. Unless otherwise specified, “6-membered saturated heterocyclyl” groups may be carbon or nitrogen linked. Ring nitrogen atoms may be optionally oxidized to form an N-oxide. Ring sulfur atoms may be optionally oxidized to form S-oxides. Illustrative examples of “6-membered saturated heterocyclyl” include, but are not limited to, 1,1-dioxidothiomorpholinyl, morpholinyl, oxopiperazinyl, piperazinyl, piperidyl, tetrahydropyranyl, and thiomorpholinyl.

Where a particular R group (e.g. R^{1a}, R¹⁰, etc.) is present in a compound of Formula (I) more than once, it is intended that each selection for that R group is independent at each occurrence of any selection at any other occurrence. For example, a group designated as —N(R²⁵)₂ group is intended to encompass: 1) those —N(R²⁵)₂ groups in which both R²⁵ substituents are the same, such as those in which both R²⁵ substituents are, for example, C₁₋₆alkyl; and 2) those —N(R²⁵)₂ groups in which each R²⁵ substituent is different, such as those in which one R²⁵ substituent is, for example, H, and the other R²⁵ substituent is, for example, carbocyclyl.

Unless specifically stated, the bonding atom of a group may be any suitable atom of that group; for example, propyl includes prop-1-yl and prop-2-yl.

Effective Amount—As used herein, the phrase “effective amount” means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician.

In particular, an effective amount of a compound of Formula (I) for use in the treatment of cancer is an amount sufficient to symptomatically relieve in a warm-blooded animal such as man, the symptoms of cancer and myeloproliferative diseases, to slow the progression of cancer and myeloproliferative diseases, or to reduce in patients with symptoms of cancer and myeloproliferative diseases the risk of getting worse.

Leaving Group—As used herein, the phrase “leaving group” is intended to refer to groups readily displaceable by a nucleophile such as an amine nucleophile, and alcohol nucleophile, or a thiol nucleophile. Examples of suitable leaving groups include halo, such as chloro and bromo, and sulfonyloxy group, such as methanesulfonyloxy and toluene-4-sulfonyloxy.

Optionally substituted—As used herein, the phrase “optionally substituted,” indicates that substitution is optional and therefore it is possible for the designated group to be either substituted or unsubstituted. In the event a substitution is desired, any number of hydrogens on the designated group may be replaced with a selection from the indicated substituents, provided that the normal valency of the atoms on a particular substituent is not exceeded, and that the substitution results in a stable compound.

In one aspect, when a particular group is designated as being optionally substituted with “one or more” substituents, the particular may be unsubstituted. In another aspect, the particular group may bear one substituent. In another aspect, the particular substituent may bear two substituents. In still another aspect, the particular group may bear three substituents. In yet another aspect, the particular group may bear four substituents. In a further aspect, the particular group may bear one or two substituents. In still a further aspect, the particular group may be unsubstituted, or may bear one or two substituents.

Pharmaceutically Acceptable—As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Protecting Group—As used herein, the term “protecting group” is intended to refer to those groups used to prevent selected reactive groups (such as carboxy, amino, hydroxy, and mercapto groups) from undergoing undesired reactions.

Illustrative examples of suitable protecting groups for a hydroxy group include acyl groups; alkanoyl groups such as acetyl; aroyl groups, such as benzoyl; silyl groups, such as trimethylsilyl; and arylmethyl groups, such as benzyl. The deprotection conditions for the above hydroxy protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

Illustrative examples of suitable protecting groups for an amino group include acyl groups; alkanoyl groups such as acetyl; alkoxycarbonyl groups, such as methoxycarbonyl, ethoxycarbonyl, and t-butoxycarbonyl; arylmethoxycarbonyl groups, such as benzyloxycarbonyl; and aroyl groups, such benzoyl. The deprotection conditions for the above amino protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric, phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid, for example boron trichloride). A suitable alternative protecting group for a primary amino group is, for example, a phthaloyl group, which may be removed by treatment with an alkylamine, for example dimethylaminopropylamine or 2-hydroxyethylamine, or with hydrazine. Another suitable protecting group for an amine is, for example, a cyclic ether such as tetrahydrofuran, which may be removed by treatment with a suitable acid such as trifluoroacetic acid.

The protecting groups may be removed at any convenient stage in the synthesis using conventional techniques well known in the chemical art, or they may be removed during a later reaction step or work-up.

The compounds of Formula (I), and of any of the examples or embodiments disclosed herein, are intended to encompass all isotopes of the atoms included therein. For example, H (or hydrogen) includes any isotopic form of hydrogen including ¹H, ²H (Deuterium), and ³H (Tritium); C includes any isotopic form of carbon including ¹²C, ¹³C, and ¹⁴C; O includes any isotopic form of oxygen including ¹⁶O, ¹⁷O and ¹⁸O; N includes any isotopic form of nitrogen including ¹³N, ¹⁴N and ¹⁵N; P includes any isotopic form of phosphorous including ³¹P and ³²P; S includes any isotopic form of sulfur including ³²S and ³⁵S; F includes any isotopic form of fluorine including ¹⁹F and ¹⁸F; Cl includes any isotopic form of chlorine including ³⁵Cl, ³⁷Cl and ³⁶Cl; and the like. It is to be understood that the invention encompasses all such isotopic forms that are useful for inhibiting JAK1 and/or JAK2 tyrosine kinases.

With reference to substituent R¹ for illustrative purposes, the following substituent definitions refer to the indicated structures:

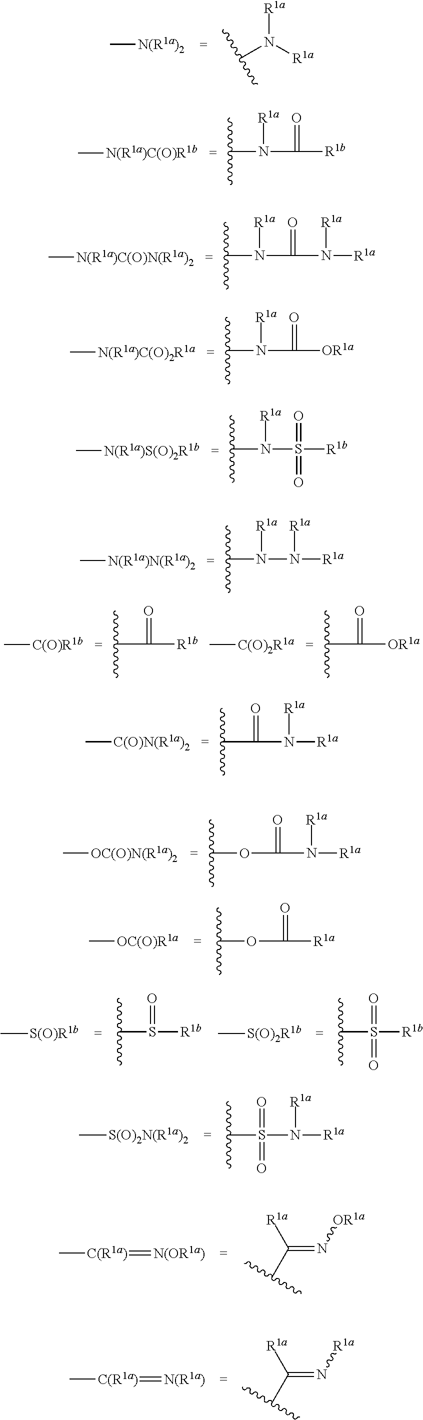

The compounds discussed herein in many instances were named or checked with ACD/Name® (Product version 10.04) by ACD/Labs®.

Compounds of Formula (I) may form stable pharmaceutically acceptable acid or base salts, and in such cases administration of a compound as a salt may be appropriate. Examples of acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Examples of base salts include ammonium salts; alkali metal salts such as sodium, lithium and potassium salts; alkaline earth metal salts such as aluminum, calcium and magnesium salts; salts with organic bases such as dicyclohexylamine salts and N-methyl-D-glucamine; and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates such as dimethyl, diethyl, dibutyl; diamyl sulfates; long chain halides such as decyl, lauryl, myristyl and stearyl halides; arylalkyl halides such as benzyl bromide and others. Non-toxic physiologically-acceptable salts are preferred, although other salts may be useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

The use of the term “salt” is intended to equally apply to the salts of enantiomers, stereoisomers, rotamers, tautomers, and racemates of the inventive compounds.

Some compounds of Formula (I) may have chiral centers and/or geometric isomeric centers (E- and Z-isomers), and it is to be understood that the invention encompasses all such optical, enantiomeric, diastereoisomeric, and/or geometric isomers. The invention further relates to any and all tautomeric forms of the compounds of Formula (I).

It is also to be understood that certain compounds of Formula (I) can exist in solvated as well as unsolvated forms such as, for example, hydrated forms. It is to be understood that the invention encompasses all such solvated forms.

Additional embodiments of the invention are as follows. These additional embodiments relate to compounds of Formula (I) and pharmaceutically acceptable salts thereof. Such specific substituents may be used, where appropriate, with any of the definitions, claims, or embodiments defined hereinbefore or hereinafter. The additional embodiments are illustrative are not to be read as limiting the scope of the invention as defined by the claims.

### Ring A

In one aspect, Ring A is selected from

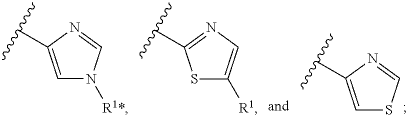

R¹ is selected from —CN and C₁₋₆alkyl;
R¹* is selected from 3- to 6-membered carbocyclyl and C₁₋₆alkyl, wherein said C₁₋₆alkyl is optionally substituted on carbon with one or more R¹⁰;
R¹⁰ in each occurrence is independently selected from halo, —CN, 3- to 6-membered carbocyclyl, 4- to 6-membered heterocyclyl, and —OR^{10a}; and
R^{10a} in each occurrence is independently selected from C₁₋₆alkyl.

In one aspect, Ring A is selected from

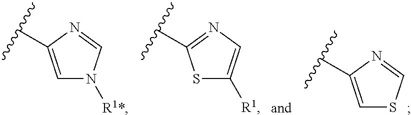

R¹ is selected from —CN and C₁₋₆alkyl;
R¹* is C₁₋₆alkyl, wherein said C₁₋₆alkyl is optionally and independently substituted on carbon with one or more R¹⁰; and
R¹⁰ in each occurrence is independently selected from 3- to 6-membered carbocyclyl, 4- to 6-membered heterocyclyl, and halo.

In another aspect, Ring A is selected from

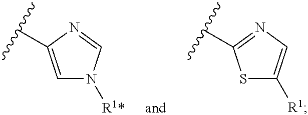

R¹ is selected from —CN and C₁₋₆alkyl, wherein said C₁₋₆alkyl is optionally substituted with one or more R¹⁰;
R¹* is C₁₋₆alkyl, wherein said C₁₋₆alkyl is optionally substituted with one or more R¹⁰; and
R¹⁰ is carbocyclyl.

In still another aspect, Ring A is

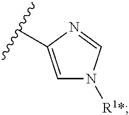

R¹* is C₁₋₆alkyl, wherein said C₁₋₆alkyl is optionally substituted with one or more R¹⁰; and
R¹⁰ is carbocyclyl.

In yet another aspect, Ring A is

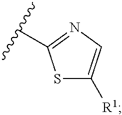

R¹ is selected from —CN and C₁₋₆alkyl, wherein said C₁₋₆alkyl is optionally substituted with one or more R¹⁰; and
R¹⁰ is carbocyclyl.

In still another aspect, Ring A is

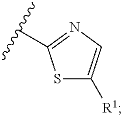

and
R¹ is selected from —CN and C₁₋₆alkyl.

In a further aspect, Ring A is selected from:

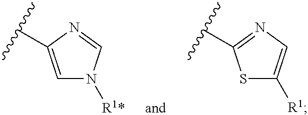

R¹ is selected from —CN and methyl, wherein said methyl is optionally substituted with one or more R¹⁰;
R¹* is selected from methyl and ethyl, wherein said methyl and ethyl are optionally substituted with one or more R¹⁰; and
R¹⁰ is phenyl.

In a further aspect, Ring A is selected from:

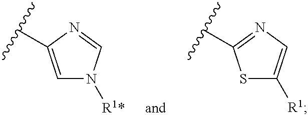

R¹ is selected from —CN and methyl;
R¹* is selected from methyl and ethyl, wherein said methyl and ethyl are optionally substituted with one or more R¹⁰; and
R¹⁰ is phenyl.

In still a further aspect, Ring A is selected from 1-(cyanomethyl)-1H-imidazol-4-yl, 5-cyano-1,3-thiazol-2-yl, 1-cyclopropyl-1H-imidazol-4-yl, 1-ethyl-1H-imidazol-4-yl, 1-isopropyl-1H-imidazol-4-yl, 1H-imidazol-4-yl, 1-(methoxymethyl)-1H-imidazol-4-yl, 1-methyl-1H-imidazol-4-yl, 5-methyl-1,3-thiazol-2-yl, 1-(2-phenylethyl)-1H-imidazol-4-yl, 1,3-thiazol-4-yl, 1-[2-(3-thienyl)ethyl]-1H-imidazol-4-yl, and 1-(2,2,2-trifluoroethyl)-1H-imidazol-4-yl.

In yet a further aspect, Ring A is selected from 5-cyano-1,3-thiazol-2-yl, 1-methyl-1H-imidazol-4-yl, 5-methyl-1,3-thiazol-2-yl, and 1-(2-phenylethyl)-1H-imidazol-4-yl.

### Ring B, R², and m

In one aspect, Ring B is 4 to 6-membered saturated heterocyclyl;

R² in each occurrence is independently selected from halo, C₁₋₆alkyl, and —OR^{2a}, wherein said C₁₋₆alkyl in each occurrence is optionally and independently substituted with one or more R²⁰;
R^{2a} is C₁₋₆alkyl;

### R²⁰ is —OH; and

m is selected from 0, 1, 2.

In another aspect, Ring B is 6-membered saturated heterocyclyl;

R² in each occurrence is independently selected from halo and C₁₋₆alkyl; and
m is selected from 0, 1, and 2.

In still another aspect, Ring B is 6-membered saturated heterocyclyl;

R² in each occurrence is independently selected from halo and C₁₋₆alkyl, wherein said C₁₋₆alkyl is in each occurrence is optionally and independently substituted with one or more R²⁰;

### R²⁰ is —OH; and

m is selected from 0, 1, and 2.

In yet another aspect, Ring B is selected from morpholinyl, piperidinyl, and azetidinyl;

R² in each occurrence is independently selected from halo, C₁₋₆alkyl, and —OR^{2a}, wherein said C₁₋₆alkyl is in each occurrence is optionally and independently substituted with one or more R²⁰;
R^{2a} is C₁₋₆alkyl;

### R²⁰ is —OH; and

m is selected from 0, 1, and 2.

In a further aspect, Ring B is selected from morpholinyl and piperidinyl;

R² in each occurrence is independently selected from halo and C₁₋₆alkyl; and
m is selected from 0, 1, and 2.

In still a further aspect, Ring B is selected from morpholinyl;

R² in each occurrence is independently selected from halo and C₁₋₆alkyl; and
m is selected from 0, 1, and 2.

In yet a further aspect, Ring B is selected from morpholinyl and piperidinyl;

R² in each occurrence is independently selected from fluoro and methyl; and
m is selected from 0, 1, and 2.

In one aspect, Ring B is selected from morpholinyl;

R² in each occurrence is independently selected from fluoro and methyl; and
m is selected from 0, 1, and 2.

In another aspect, Ring B is selected from morpholin-4-yl and piperidin-1-yl;

R² in each occurrence is independently selected from halo and C₁₋₆alkyl; and
m is selected from 0, 1, and 2.

In still another aspect, Ring B is morpholin-4-yl and piperidin-1-yl;

R² in each occurrence is independently selected from fluoro and methyl; and
m is selected from 0, 1, and 2.

In yet another aspect, Ring B is morpholin-4-yl;

R² in each occurrence is independently selected from fluoro and methyl; and
m is selected from 0, 1, and 2.

In a further aspect, Ring B, R², and m together form a group selected from 4,4-difluoropiperidin-1-yl, 2,2-dimethylmorpholin-4-yl, 2,6-dimethylmorpholin-4-yl, 2-methylmorpholin-4-yl, 3-fluoroazetidin-1-yl, 4-fluoropiperidin-1-yl, 3-(hydroxymethyl)morpholin-4-yl, 3-methoxyazetidin-1-yl, and morpholin-4-yl.

In still a further aspect, Ring B, R², and m together form a group selected from 4,4-difluoropiperidin-1-yl, 2,2-dimethylmorpholin-4-yl, 2,6-dimethylmorpholin-4-yl, 2-methylmorpholin-4-yl, and morpholin-4-yl.

### Ring C, R⁴, and n

In one aspect, Ring C is selected from phenyl and 6-membered heteroaryl;

R⁴ in each occurrence is independently selected from halo and —CN; and
n is selected from 1 and 2.

In another aspect, Ring C is selected from pyridinyl and pyrimidinyl;

R⁴ is halo; and
n is selected from 1 and 2.

In still another aspect, Ring C is selected from phenyl, pyridinyl, and pyrimidinyl;

R⁴ is halo; and
n is selected from 1 and 2.

In yet another aspect, Ring C is selected from pyridinyl and pyrimidinyl;

R⁴ is fluoro; and
n is selected from 1 and 2.

In a further aspect, Ring C is selected from phenyl, pyridinyl, and pyrimidinyl;

R⁴ is selected from fluoro, chloro, and —CN; and
n is selected from 1 and 2.

In still a further aspect, Ring C is selected from pyridin-2-yl and pyrimidin-2-yl;

R⁴ is fluoro; and
n is selected from 1 and 2.

In yet a further aspect, Ring C, R⁴, and n together form a group selected from 4-chlorophenyl, 4-cyanophenyl, 3,5-difluoropyridin-2-yl, 4-fluorophenyl, and 5-fluoropyrimidin-2-yl.

In one aspect, Ring C, R⁴, and n together form a group selected from 3,5-difluoropyridin-2-yl and 5-fluoropyrimidin-2-yl.

In another aspect, Ring C, R⁴, and n together form 3,5-difluoropyridin-2-yl.

In still another aspect, Ring C, R⁴, and n together form 5-fluoropyrimidin-2-yl.

### R³

In one aspect, R³ is selected from C₁₋₆alkyl, 3- to 6-membered carbocyclyl, and 4- to 6-membered heterocyclyl, wherein said C₁₋₆alkyl is optionally substituted with one or more R³⁰, and wherein any —NH— moiety of said 4- to 6-membered heterocyclyl is optionally substituted with R³⁰*;

### R³⁰ is —OR^{30a};

R³⁰* is C₁₋₆alkyl; and
R^{30a} is C₁₋₆alkyl.

In another aspect, R³ is C₁₋₆alkyl, wherein said C₁₋₆alkyl is optionally substituted with one or more R³⁰;

### R³⁰ is —OR^{30a}; and

R^{30a} is C₁₋₆alkyl.

In still another aspect, R³ is methyl, wherein said methyl is optionally substituted with one or more R³⁰;

### R³⁰ is —OR^{30a}; and

R^{30a} is C₁₋₆alkyl.

In yet another aspect, R³ is methyl, wherein said methyl is optionally substituted with one or more R³⁰;

### R³⁰ is —OR^{30a}; and

R^{30a} is methyl.

In a further aspect, R³ is selected from cyclopentyl, methoxymethyl, methyl, and 1-methyl-1H-imidazol-4-yl.

In still a further aspect, R³ is selected from methyl and methoxymethyl.

In yet further aspect, R³ is methyl.

### R⁴

In one aspect, R⁴ is halo.

In another aspect, R⁴ is fluoro.

m

In one aspect, m is selected from 0, 1, and 2.

n

In one aspect, n is selected from 1 and 2.

### Ring A, Ring B, Ring C, R², R³, R⁴, m, and n

In one aspect, Ring A is selected from:

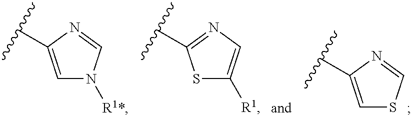

Ring B is 4 to 8-membered saturated heterocyclyl;
Ring C is selected from phenyl and 6-membered heteroaryl;
R¹ is selected from H, halo, —CN, C₁₋₆alkyl, C₂₋₆alkenyl, C₂₋₆alkynyl, carbocyclyl, heterocyclyl, —OR^{1a}, —SR^{1a}, —N(R^{1a})₂, —N(R^{1a})C(O)R^{1b}, —N(R^{1a})N(R^{1a})₂, —NO₂, —N(R^{1a})OR^{1a}, —ON(R^{1a})₂, —C(O)H, —C(O)R^{1b}, —C(O)₂R^{1a}, —C(O)N(R^{1a})₂, —C(O)N(R^{1a})(OR^{1a}), —OC(O)N(R^{1a})₂, —N(R^{1a})C(O)₂R^{1a}, —N(R^{1a})C(O)N(R^{1a})₂, —OC(O)R^{1b}, —S(O)R^{1b}, —S(O)₂R^{1b}, —S(O)₂N(R^{1a})₂, —N(R^{1a})S(O)₂R^{1b}, —C(R^{1a})═N(R^{1a}), and —C(R^{1a})═N(OR^{1a}), wherein said C₁₋₆alkyl, C₂₋₆alkenyl, C₂₋₆alkynyl, carbocyclyl, and heterocyclyl are optionally substituted on carbon with one or more R¹⁰, and wherein any —NH— moiety of said heterocyclyl is optionally substituted with R¹⁰*;
R^{1a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
R^{1b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R¹⁰, and wherein any —NH— moiety of said heterocyclyl is optionally substituted with R¹⁰*;
R^{1c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
R¹* is selected from H, —CNC₁₋₆alkyl, carbocyclyl, heterocyclyl, —OR^{1a}, —C(O)H, —C(O)R^{1b}, —C(O)₂R^{1c}, —C(O)N(R^{1a})₂, —S(O)R^{1b}, —S(O)₂R^{1b}, —S(O)₂N(R^{1a})₂, —C(R^{10a})═N(R^{1a}), and —C(R^{1a})═N(OR^{1a}), wherein said C₁₋₆alkyl, carbocyclyl, and heterocyclyl are optionally substituted on carbon with one or more R¹⁰, and wherein any —NH— moiety of said heterocyclyl is optionally substituted with R¹⁰*;
R² in each occurrence is independently selected from halo, —CN, C₁₋₆alkyl, C₂₋₆alkenyl, C₂₋₆alkynyl, carbocyclyl, heterocyclyl, —OR^{2a}, —SR^{2a}, N(R^{2a})₂, N(R^{2a})C(O)R^{2b}, —N(R^{2a})N(R^{2a})₂, —NO₂, —N(R^{2a})OR^{2a}, —ON(R^{2a})₂, —C(O)H, —C(O)R^{2b}, —C(O)₂R^{2a}, —C(O)N(R^{2a})₂, —C(O)N(R^{2a})(OR^{2a})—OC(O)N(R^{2a})₂, —N(R^{2a})C(O)₂R^{2a}, —N(R^{2a})C(O)N(R^{2a})₂, —OC(O)R^{2b}, —S(O)R^{2b}, —S(O)₂R^{2b}, —S(O)₂N(R^{2a})₂, —N(R^{2a})S(O)₂R^{2b}, —C(R^{2a})═N(R^{2a}), and —C(R^{2a})═N(OR^{2a});
R^{2a} in each occurrence is independently selected from H, C₁₋₆alkyl, carbocyclyl, and heterocyclyl;
R^{2b} in each occurrence is independently selected from C₁₋₆alkyl, C₂₋₆alkenyl, C₂₋₆alkynyl, carbocyclyl, and heterocyclyl;
R³ is selected from H, halo, —CN, C₁₋₆alkyl, C₂₋₆alkenyl, C₂₋₆alkynyl, carbocyclyl, heterocyclyl, —OR^{3a}, —SR^{3a}, —N(R^{3a})₂, —N(R^{3a})C(O)R^{3b}, —N(R^{3a})N(R^{3a})₂, —NO₂, —N(R^{3a})—OR^{3a}, —O—N(R^{3a})₂, —C(O)H, —C(O)R^{3b}, —C(O)₂R^{3a}, —C(O)N(R^{3a})₂, —C(O)N(R^{3a})(OR^{3a}), —OC(O)N(R^{3a})₂, —N(R^{3a})C(O)₂R³, —N(R^{3a})C(O)N(R^{3a})₂, —OC(O)R^{3b}, —S(O)R^{3b}, —S(O)₂R^{3b}, —S(O)₂N(R^{3a})₂, —N(R^{3a})S(O)₂R^{3b}, —C(R^{3a})═N(R^{3a}), and —C(R^{3a})═N(OR^{3a}), wherein said C₁₋₆alkyl, C₂₋₆alkenyl, C₂₋₆alkynyl, carbocyclyl, and heterocyclyl are optionally substituted on carbon with one or more R³⁰, and wherein any —NH— moiety of said heterocyclyl is optionally substituted with R³⁰*;
R^{3a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R³⁰, and wherein any —NH— moiety of said heterocyclyl is optionally substituted with R³⁰*;

R^{3b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R³⁰, and wherein any —NH— moiety of said heterocyclyl is optionally substituted with R³⁰*;

R⁴ in each occurrence is independently selected from halo, —CN, C₁₋₆alkyl, C₂₋₆alkenyl, C₂₋₆alkynyl, carbocyclyl, heterocyclyl, —OR^{4a}, —SR^{4a}, —N(R^{4a})₂, —N(R^{4a})C(O)R^{4b}, —N(R^{4a})N(R^{4a})₂, —NO₂, —N(R^{4a})—OR^{4a}, —O—N(R^{4a})₂, —C(O)H, —C(O)R^{4b}, —C(O)₂R^{4a}, —C(O)N(R^{4a})₂, —C(O)N(R^{4a})(OR^{4a})—OC(O)N(R^{4a})₂, —N(R^{4a})C(O)₂R^{4a}, —N(R^{4a})C(O)N(R^{4a})₂, —OC(O)R^{4b}, —S(O)R^{4b}, —S(O)₂R^{4b}, —S(O)₂N(R^{4a})₂, —N(R^{4a})S(O)₂R^{4b}, —C(R^{4a})═N(R^{4a}), and —C(R^{4a})═N(OR^{4a});
R^{4a} in each occurrence is independently selected from H, C₁₋₆alkyl, carbocyclyl, and heterocyclyl;
R^{4b} in each occurrence is independently selected from C₁₋₆alkyl, C₂₋₆alkenyl, C₂₋₆alkynyl, carbocyclyl, and heterocyclyl;
R¹⁰ in each occurrence is independently selected from halo, —CN, C₁₋₆alkyl, C₂₋₆alkenyl, C₂₋₆alkynyl, carbocyclyl, heterocyclyl, —OR^{10a}, —SR^{10a}, —N(R^{10a})₂, —N(R^{10a})C(O)R^{10b}, —N(R^{10a})N(R^{10a})₂, —NO₂, —N(R^{10a})—OR^{10a}, —O—N(R^{10a})₂, —C(O)H, —C(O)R^{10b}, —C(O)₂R^{10a}, —C(O)N(R^{10a})₂, —C(O)N(R^{10a})(OR^{10a}), —OC(O)N(R^{10a})₂, —N(R^{10a})C(O)₂R^{10a}, —N(R^{10a})C(O)N(R^{10a})₂, —OC(O)R^{10b}, —S(O)R^{10b}, —S(O)₂R^{10b}, —S(O)₂N(R^{10a})₂, —N(R^{10a})S(O)₂R^{10b}, —C(R^{10a})═N(R^{10a}), and —C(R^{10a})═N(OR^{10a});
R¹⁰* in each occurrence is independently selected from C₁₋₆alkyl, carbocyclyl, heterocyclyl, —C(O)H, —C(O)R^{10b}, —C(O)₂R^{10c}, —C(O)N(R^{10a})₂, —S(O)R^{10b}, —S(O)₂R^{10b}, —S(O)₂N(R^{10a})₂, —C(R^{10a})═N(R^{10a}), and —C(R^{10a})═N(OR^{10a});
R^{10a} in each occurrence is independently selected from H, C₁₋₆alkyl, carbocyclyl, and heterocyclyl;
R^{10b} in each occurrence is independently selected from C₁₋₆alkyl, C₂₋₆alkenyl, C₂₋₆alkynyl, carbocyclyl, and heterocyclyl*;
R^{10c} in each occurrence is independently selected from C₁₋₆alkyl, carbocyclyl, and heterocyclyl;
R³⁰ in each occurrence is independently selected from halo, —CN, C₁₋₆alkyl, C₂₋₆alkenyl, C₂₋₆alkynyl, carbocyclyl, heterocyclyl, —OR^{30a}, —SR^{30a}, —N(R^{30a})₂, —N(R^{30a})C(O)R^{30b}, —N(R^{30a})N(R^{30a})₂, —NO₂, —N(R^{30a})—OR^{30a}, —O—N(R^{30a})₂, —C(O)H, —C(O)R^{30b}, —C(O)₂R^{30a}, —C(O)N(R^{30a})₂, —C(O)N(R^{30a})(OR^{30a}), —OC(O)N(R^{30a})₂, —N(R^{30a})C(O)₂R^{30a}, —N(R^{30a})C(O)N(R^{30a})₂, —OC(O)R^{30b}, —S(O)R^{30b}, —S(O)₂R^{30b}, —S(O)₂N(R^{30a})₂, —N(R^{30a})S(O)₂R^{30b}, —C(R^{30a})═N(R^{30a}), and —C(R^{30a})═N(OR^{30a});
R³⁰* in each occurrence is independently selected from C₁₋₆alkyl, carbocyclyl, heterocyclyl, —C(O)H, —C(O)R^{30b}, —C(O)₂R^{30c}, —C(O)N(R^{30a})₂, —S(O)R^{30b}, —S(O)₂R^{30b}, —S(O)₂N(R^{30a})₂, —C(R^{30a})═N(R^{30a}), and —C(R^{30a})═N(OR^{30a});
R^{30a} in each occurrence is independently selected from H, C₁₋₆alkyl, carbocyclyl, and heterocyclyl;
R^{30b} in each occurrence is independently selected from C₁₋₆alkyl, C₂₋₆alkenyl, C₂₋₆alkynyl, carbocyclyl, and heterocyclyl;
R^{30c} in each occurrence is independently selected from C₁₋₆alkyl, carbocyclyl, and heterocyclyl;
m is selected from 0, 1, and 2; and
n is selected from 1 and 2.

In another aspect, Ring A is selected from

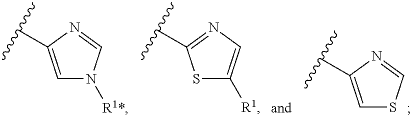

Ring B is 4 to 6-membered saturated heterocyclyl;
Ring C is selected from phenyl and 6-membered heteroaryl;
R¹ is selected from —CN and C₁₋₆alkyl;
R¹* is selected from 3- to 6-membered carbocyclyl and C₁₋₆alkyl, wherein said C₁₋₆alkyl is optionally substituted on carbon with one or more R¹⁰;
R² in each occurrence is independently selected from halo, C₁₋₆alkyl, and —OR^{2a}, wherein said C₁₋₆alkyl in each occurrence is optionally and independently substituted with one or more R²⁰;
R^{2a} is C₁₋₆alkyl;
R³ is selected from C₁₋₆alkyl, 3 to 6-membered carbocyclyl, and 4 to 6-membered heterocyclyl, wherein said C₁₋₆alkyl is optionally substituted with one or more R³⁰, and wherein any —NH— moiety of said 4 to 6-membered heterocyclyl is optionally substituted with R³⁰*;
R⁴ in each occurrence is independently selected from halo and —CN;
R¹⁰ in each occurrence is independently selected from halo, —CN, 3- to 6-membered carbocyclyl, 4- to 6-membered heterocyclyl, and —OR^{10a};
R^{10a} is C₁₋₆alkyl;

### R²⁰ is —OH;

### R³⁰ is —OR^{30a};

R³⁰* is C₁₋₆alkyl;
R^{30a} is C₁₋₆alkyl;
m is selected from 0, 1, 2; and
n is selected from 1 and 2.

In still another aspect, Ring A is selected from:

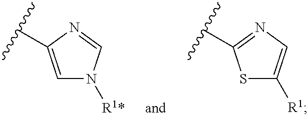

Ring B is 6-membered saturated heterocyclyl;
Ring C is selected from pyridinyl and pyrimidinyl;
R¹ is selected from —CN and C₁₋₆alkyl, wherein said C₁₋₆alkyl is optionally substituted with one or more R¹⁰;
R¹* is C₁₋₆alkyl, wherein said C₁₋₆alkyl is optionally substituted with one or more R¹⁰;
R² in each occurrence is independently selected from halo and C₁₋₆alkyl;
R³ is C₁₋₆alkyl, wherein said C₁₋₆alkyl is optionally substituted with one or more R³⁰;
R⁴ is halo;
R¹⁰ is carbocyclyl;

### R³⁰ is —OR^{30a};

R^{30a} is C₁₋₆alkyl;
m is selected from 0, 1, and 2; and
n is selected from 1 and 2.

In yet another aspect, Ring A is selected from:

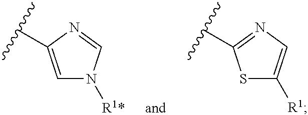

Ring B is selected from morpholinyl and piperidinyl;
Ring C is selected from pyridinyl and pyrimidinyl;
R¹ is selected from —CN and C₁₋₆alkyl, wherein said C₁₋₆alkyl is optionally substituted with one or more R¹⁰;
R¹* is C₁₋₆alkyl, wherein said C₁₋₆alkyl is optionally substituted with one or more R¹⁰;
R² in each occurrence is independently selected from halo and C₁₋₆alkyl;
R³ is C₁₋₆alkyl, wherein said C₁₋₆alkyl is optionally substituted with one or more R³⁰;
R⁴ is halo;
R¹⁰ is carbocyclyl;

### R³⁰ is —OR^{30a};

R^{30a} is C₁₋₆alkyl;
m is selected from 0, 1, and 2; and
n is selected from 1 and 2.

In a further aspect, Ring A is selected from:

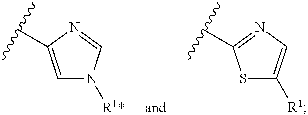

Ring B is selected from morpholinyl and piperidinyl;
Ring C is selected from pyridinyl and pyrimidinyl;
R¹ is selected from —CN and methyl, wherein said methyl is optionally substituted with one or more R¹⁰;
R¹* is selected from methyl and ethyl, wherein said methyl and ethyl are optionally substituted with one or more R¹⁰;
R² in each occurrence is independently selected from fluoro and methyl;
R³ is methyl, wherein said methyl is optionally substituted with one or more R³⁰;
R⁴ is fluoro;
R¹⁰ is phenyl;

### R³⁰ is —OR^{30a};

R^{30a} is methyl;
m is selected from 0, 1, and 2; and
n is selected from 1 and 2.

In still a further aspect, Ring A is selected from 1-(cyanomethyl)-1H-imidazol-4-yl, 5-cyano-1,3-thiazol-2-yl, 1-cyclopropyl-1H-imidazol-4-yl, 1-ethyl-1H-imidazol-4-yl, 1-isopropyl-1H-imidazol-4-yl, 1H-imidazol-4-yl, 1-(methoxymethyl)-1H-imidazol-4-yl, 1-methyl-1H-imidazol-4-yl, 5-methyl-1,3-thiazol-2-yl, 1-(2-phenylethyl)-1H-imidazol-4-yl, 1,3-thiazol-4-yl, 1-[2-(3-thienyl)ethyl]-1H-imidazol-4-yl, and 1-(2,2,2-trifluoroethyl)-1H-imidazol-4-yl;

Ring B, R², and m together form a group selected from 4,4-difluoropiperidin-1-yl, 2,2-dimethylmorpholin-4-yl, 2,6-dimethylmorpholin-4-yl, 2-methylmorpholin-4-yl, 3-fluoroazetidin-1-yl, 4-fluoropiperidin-1-yl, 3-(hydroxymethyl)morpholin-4-yl, 3-methoxyazetidin-1-yl, and morpholin-4-yl;
Ring C, R⁴, and n form a group selected from 4-chlorophenyl, 4-cyanophenyl, 3,5-difluoropyridin-2-yl, 4-fluorophenyl, and 5-fluoropyrimidin-2-yl; and
R³ is selected from cyclopentyl, methoxymethyl, methyl, and 1-methyl-1H-imidazol-4-yl.

In yet a further aspect, Ring A is selected from 5-cyano-1,3-thiazol-2-yl, 1-methyl-1H-imidazol-4-yl, 5-methyl-1,3-thiazol-2-yl, and 1-(2-phenylethyl)-1H-imidazol-4-yl;

Ring B, R², and m together form a group selected from 4,4-difluoropiperidin-1-yl, 2,2-dimethylmorpholin-4-yl, 2,6-dimethylmorpholin-4-yl, 2-methylmorpholin-4-yl, and morpholin-4-yl;
Ring C, R⁴, and n together form a group selected from 3,5-difluoropyridin-2-yl and 5-fluoropyrimidin-2-yl; and
R³ is selected from methyl and methoxymethyl.

In yet a further aspect, the compounds of Formula (I) may be compounds of Formula (Ia):

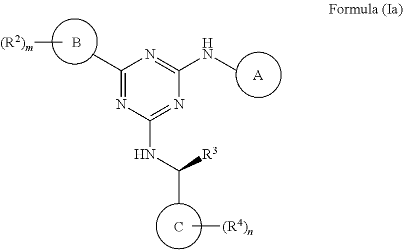

or pharmaceutically acceptable salts thereof, wherein Ring A, Ring B, Ring C, R², R³, R⁴, m, and n are as defined hereinabove.

In one aspect, the present invention provides compounds of Formula (I), or pharmaceutically acceptable salts thereof, as illustrated by the Examples, each of which provides a further independent aspect of the invention.

In another aspect, the present invention provides a compound selected from:
N-[(1R)-1-(3,5-Difluoropyridin-2-yl)-2-methoxyethyl]-6-[(2R,6S)-2,6-dimethylmorpholin-4-yl]-N′-(1-methyl-1H-imidazol-4-yl)-1,3,5-triazine-2,4-diamine;
N-[(1R)-1-(3,5-Difluoropyridin-2-yl)-2-methoxyethyl]-N′-(1-methyl-1H-imidazol-4-yl)-6-(2-methylmorpholin-4-yl)-1,3,5-triazine-2,4-diamine;
N-[(1R)-1-(3,5-Difluoropyridin-2-yl)-2-methoxyethyl]-6-(2,2-dimethylmorpholin-4-yl)-N-(1-methyl-1H-imidazol-4-yl)-1,3,5-triazine-2,4-diamine;
N-[(1R)-1-(3,5-Difluoropyridin-2-yl)-2-methoxyethyl]-N′-(1-methyl-1H-imidazol-4-yl)-6-morpholin-4-yl-1,3,5-triazine-2,4-diamine;
N-[(1S)-1-(5-Fluoropyrimidin-2-yl)ethyl]-N′-(1-methyl-1H-imidazol-4-yl)-6-morpholin-4-yl-1,3,5-triazine-2,4-diamine;
N-[(1S)-1-(5-Fluoropyrimidin-2-yl)ethyl]-6-morpholin-4-yl-N′-[1-(2-phenylethyl)-1H-imidazol-4-yl]-1,3,5-triazine-2,4-diamine;
2-[(4-{[(1S)-1-(5-Fluoropyrimidin-2-yl)ethyl]amino}-6-morpholin-4-yl-1,3,5-triazin-2-yl)amino]-1,3-thiazole-5-carbonitrile;
N-[(1S)-1-(5-Fluoropyrimidin-2-yl)ethyl]-N′-(5-methyl-1,3-thiazol-2-yl)-6-morpholin-4-yl-1,3,5-triazine-2,4-diamine;
6-(4,4-Difluoropiperidin-1-yl)-N′-[(1S)-1-(3,5-difluoropyridin-2-yl)ethyl]-N′-(1-methyl-1H-imidazol-4-yl)-1,3,5-triazine-2,4-diamine;
N-[1-(3,5-Difluoropyridin-2-yl)ethyl]-N′-(1-methyl-1H-imidazol-4-yl)-6-morpholin-4-yl-1,3,5-triazine-2,4-diamine;
N-[(1R)-1-(3,5-Difluoropyridin-2-yl)ethyl]-N′-(1-methyl-1H-imidazol-4-yl)-6-morpholin-4-yl-1,3,5-triazine-2,4-diamine;
N-[(1S)-1-(3,5-Difluoropyridin-2-yl)ethyl]-N′-(1-methyl-1H-imidazol-4-yl)-6-morpholin-4-yl-1,3,5-triazine-2,4-diamine;
N-[1-(3,5-Difluoropyridin-2-yl)ethyl]-N′-(1-methyl-1H-imidazol-4-yl)-6-(²H₈)morpholin-4-yl-1,3,5-triazine-2,4-diamine;
N-[(1R)-1-(3,5-Difluoropyridin-2-yl)ethyl]-N′-(1-methyl-1H-imidazol-4-yl)-6-(²H₈)morpholin-4-yl-1,3,5-triazine-2,4-diamine;
N-[(1S)-1-(3,5-Difluoropyridin-2-yl)ethyl]-N′-(1-methyl-1H-imidazol-4-yl)-6-(²H₈)morpholin-4-yl-1,3,5-triazine-2,4-diamine;
N-[1-(3,5-Difluoropyridin-2-yl)ethyl]-N′-[1-(²H₃)methyl-1H-imidazol-4-yl]-6-morpholin-4-yl-1,3,5-triazine-2,4-diamine;
N-[(1R)-1-(3,5-Difluoropyridin-2-yl)ethyl]-N′-[1-(²H₃)methyl-1H-imidazol-4-yl]-6-morpholin-4-yl-1,3,5-triazine-2,4-diamine;
N-[(1S)-1-(3,5-Difluoropyridin-2-yl)ethyl]-N′-[1-(²H₃)methyl-1H-imidazol-4-yl]-6-morpholin-4-yl-1,3,5-triazine-2,4-diamine;
N-[1-(3,5-Difluoropyridin-2-yl)ethyl]-N′-[1-(²H₃)methyl-1H-imidazol-4-yl]-6-(²H₈)morpholin-4-yl-1,3,5-triazine-2,4-diamine;
N-[(1R)-1-(3,5-Difluoropyridin-2-yl)ethyl]-N′-[1-(²H₃)methyl-1H-imidazol-4-yl]-6-(²H₈)morpholin-4-yl-1,3,5-triazine-2,4-diamine;
N-[(1S)-1-(3,5-Difluoropyridin-2-yl)ethyl]-N′-[1-(²H₃)methyl-1H-imidazol-4-yl]-6-(²H₈)morpholin-4-yl-1,3,5-triazine-2,4-diamine;
6-(4,4-Difluoropiperidin-1-yl)-N′-[(1S)-1-(5-fluoropyrimidin-2-yl)ethyl]-N′-(1-methyl-1H-imidazol-4-yl)-1,3,5-triazine-2,4-diamine;
{4-[(4-{[(1S)-1-(5-Fluoropyrimidin-2-yl)ethyl]amino}-6-morpholin-4-yl-1,3,5-triazin-2-yl)amino]-1H-imidazol-1-yl}acetonitrile;
N-[(1S)-1-(5-Fluoropyrimidin-2-yl)ethyl]-N′-[1-(methoxymethyl)-1H-imidazol-4-yl]-6-morpholin-4-yl-1,3,5-triazine-2,4-diamine;
N-[(1S)-1-(5-Fluoropyrimidin-2-yl)ethyl]-N′-(1-isopropyl-1H-imidazol-4-yl)-6-morpholin-4-yl-1,3,5-triazine-2,4-diamine;
N-[(1S)-1-(3,5-Difluoropyridin-2-yl)ethyl]-6-(3-fluoroazetidin-1-yl)-N′-(1-methyl-1H-imidazol-4-yl)-1,3,5-triazine-2,4-diamine;
N-[(1S)-1-(3,5-Difluoropyridin-2-yl)ethyl]-6-(3-methoxyazetidin-1-yl)-N′-(1-methyl-1H-imidazol-4-yl)-1,3,5-triazine-2,4-diamine;
N-[(1S)-1-(5-Fluoropyrimidin-2-yl)ethyl]-6-(3-methoxyazetidin-1-yl)-N′-(1-methyl-1H-imidazol-4-yl)-1,3,5-triazine-2,4-diamine;
N-[(1S)-1-(3,5-Difluoropyridin-2-yl)ethyl]-6-(4-fluoropiperidin-1-yl)-N′-(1-methyl-1H-imidazol-4-yl)-1,3,5-triazine-2,4-diamine;
[(3R)-4-(4-{[(1S)-1-(3,5-difluoropyridin-2-yl)ethyl]amino}-6-[(1-methyl-1H-imidazol-4-yl)amino]-1,3,5-triazin-2-yl)morpholin-3-yl]methanol;
N-[(1S)-1-(5-Fluoropyrimidin-2-yl)ethyl]-N′-1H-imidazol-4-yl-6-morpholin-4-yl-1,3,5-triazine-2,4-diamine;
tert-Butyl [2-(4-fluorophenyl)-2-({4-[(1-methyl-1H-imidazol-4-yl)amino]-6-morpholin-4-yl-1,3,5-triazin-2-yl}amino)ethyl]carbamate;
tert-Butyl [(2R)-2-(4-fluorophenyl)-2-({4-[(1-methyl-1H-imidazol-4-yl)amino]-6-morpholin-4-yl-1,3,5-triazin-2-yl}amino)ethyl]carbamate;
tert-Butyl [(2S)-2-(4-fluorophenyl)-2-({4-[(1-methyl-1H-imidazol-4-yl)amino]-6-morpholin-4-yl-1,3,5-triazin-2-yl}amino)ethyl]carbamate;
N-[(4-Fluorophenyl)(1-methyl-1H-imidazol-2-yl)methyl]-N′-(1-methyl-1H-imidazol-4-yl)-6-morpholin-4-yl-1,3,5-triazine-2,4-diamine;
N—[(R)-(4-Fluorophenyl)(1-methyl-1H-imidazol-2-yl)methyl]-N′-(1-methyl-1H-imidazol-4-yl)-6-morpholin-4-yl-1,3,5-triazine-2,4-diamine N—[(S)-(4-Fluorophenyl)(1-methyl-1H-imidazol-2-yl)methyl]-N′-(1-methyl-1H-imidazol-4-yl)-6-morpholin-4-yl-1,3,5-triazine-2,4-diamine;
N-[(1S)-1-(5-Fluoropyrimidin-2-yl)ethyl]-6-morpholin-4-yl-N′-1,3-thiazol-4-yl-1,3,5-triazine-2,4-diamine;
N-[Cyclopentyl(4-fluorophenyl)methyl]-N′-(1-methyl-1H-imidazol-4-yl)-6-morpholin-4-yl-1,3,5-triazine-2,4-diamine;
4-[(1S)-1-({4-[(1-methyl-1H-imidazol-4-yl)amino]-6-morpholin-4-yl-1,3,5-triazin-2-yl}amino)ethyl]benzonitrile;
N-[(1S)-1-(4-Chlorophenyl)ethyl]-N′-(1-methyl-1H-imidazol-4-yl)-6-morpholin-4-yl-1,3,5-triazine-2,4-diamine;
N-[(1S)-1-(4-fluorophenyl)ethyl]-N′-(1-methyl-1H-imidazol-4-yl)-6-morpholin-4-yl-1,3,5-triazine-2,4-diamine;
N-[(1S)-1-(3,5-difluoropyridin-2-yl)ethyl]-N′-(1-ethyl-1H-imidazol-4-yl)-6-morpholin-4-yl-1,3,5-triazine-2,4-diamine;
N-(1-Cyclopropyl-1H-imidazol-4-yl)-N′-[(1S)-1-(5-fluoropyrimidin-2-yl)ethyl]-6-morpholin-4-yl-1,3,5-triazine-2,4-diamine;
N-[(1S)-1-(5-Fluoropyrimidin-2-yl)ethyl]-6-morpholin-4-yl-N′-{1-[2-(3-thienyl)ethyl]-1H-imidazol-4-yl}-1,3,5-triazine-2,4-diamine;
N-[(1S)-1-(5-fluoropyrimidin-2-yl)ethyl]-6-morpholin-4-yl-N′-[1-(2,2,2-trifluoroethyl)-1H-imidazol-4-yl]-1,3,5-triazine-2,4-diamine; and N-(1-Ethyl-1H-imidazol-4-yl)-N′-[(1S)-1-(5-fluoropyrimidin-2-yl)ethyl]-6-morpholin-4-yl-1,3,5-triazine-2,4-diamine,
   or a pharmaceutically acceptable salt thereof.

### Utility

### JAK1

The compounds of Formula (I) are believed to be useful for inhibiting tyrosine kinases, particularly the JAK family and more particularly JAK1.

JAK1 activity is involved in a variety of human cancers such as acute lymphoblastic leukemia, acute myeloid leukemia, inflammatory hepatocellular adenoma and cancer related processes. Thus, inhibitors of tyrosine kinase, particularly the JAK family and more particularly JAK1, are expected to be active against neoplastic disease such as carcinoma of the breast, ovary, lung, colon, prostate or other tissues, as well as leukemias, myelomas and lymphomas, tumors of the central and peripheral nervous system, and other tumor types such as melanoma, fibrosarcoma and osteosarcoma.

Tyrosine kinase inhibitors, particularly the JAK family inhibitors and more particularly JAK1 inhibitors are also expected to be useful for the treatment other proliferative diseases including but not limited to autoimmune, inflammatory, neurological, and cardiovascular diseases.

The compounds of Formula (I) should also be useful as standards and reagents in determining the ability of a potential pharmaceutical to inhibit tyrosine kinases, particularly the JAK family and more particularly JAK1. These would be provided in commercial kits comprising a compound of this invention.

### Method 1 (JAK1)

Janus kinase 1 (JAK1) activity may be determined by measuring the kinase's ability to phosphorylate a tyrosine residue within a peptide substrate using a mobility shift assay on a Caliper LC3000 reader (Caliper, Hopkinton, Mass.), which measures fluorescence of the phosphorylated and unphosphorylated substrate and calculates a ratiometric value to determine percent turnover.

To measure JAK1 kinase activity, a commercially available purified enzyme may be used. The enzyme may be a recombinant human, catalytic domain (amino acids 866-1154), GST-tagged, expressed in insect cells (Invitrogen, Carlsbad, Calif.). After incubation of the kinase with a FITC labeled JAK1 substrate, adenosine triphosphate (ATP), and MgCl₂ for 90 minutes at room temperature, the kinase reaction may be stopped by the addition of 36 mM ethylenediaminetetraacetic acid (EDTA). The reaction may be performed in 384 well microtitre plates and the reaction products may be detected using the Caliper LC3000 Reader.

| | |
|---|---|
| Peptide substrate | FITC-C6-KKHTDDGYMPMSPGVA-NH2 |
| | (Intonation, Boston, MA) |
| ATP Km | 55 μM |
| Assay conditions | 3.5 nM JAK1 enzyme, 5 mM ATP, 1 μM JAK1 |
| | substrate, 10 mM MgCl₂, 50 mM HEPES buffer |
| | (pH 7.3), 1 mM DTT, 0.01% Tween 20, 50 |
| | μg/ml BSA |
| Incubation | 90 minutes, room temperature |
| Termination/ | 65 mM HEPES, 36 mM EDTA, 0.2% Coatin |
| Detection | Reagent 3 (Caliper, Hopkinton, MA), 0.003% |
| conditions | Tween 20 |
| Caliper LC3000 | −1.2 PSI, −2100 V downstream voltage, −1000 V |
| settings | upstream voltage, 0.2 second sample sip time, 50 |
| | second post sip time, 10% laser strength. |

When tested in an in-vitro assay based on the one described for Method 1 (JAK1) above, the JAK inhibitory activity of the following examples were measured at the indicated IC₅₀ values.

| Ex | IC₅₀ (μM) |
|---|---|
| 11a | 0.78 |
| 11b | 0.015 |
| 24a | 0.083 |
| 24b | 1.02 |
| 25b | 30 |
| 27 | 1.98 |
| 29 | 0.51 |
| 30 | 0.065 |

### JAK2

The compounds of Formula (I) are believed to be useful for inhibiting tyrosine kinases, particularly the JAK family and more particularly JAK2.

The compounds of Formula (I) are useful for the treatment of myeloproliferative disorders, myelodysplastic syndrome and cancer by inhibiting the tyrosine kinases, particularly the JAK family and more particularly JAK2. Methods of treatment target tyrosine kinase activity, particularly the JAK family activity and more particularly JAK2 activity, which is involved in a variety of myeloproliferative disorders, myelodysplastic syndrome and cancer related processes. Thus, inhibitors of tyrosine kinase, particularly the JAK family and more particularly JAK2, are expected to be active against myeloproliferative disorders such as chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and neoplastic disease such as carcinoma of the breast, ovary, lung, colon, prostate or other tissues, as well as leukemias, myelomas and lymphomas, tumors of the central and peripheral nervous system, and other tumor types such as melanoma, fibrosarcoma and osteosarcoma. Tyrosine kinase inhibitors, particularly the JAK family inhibitors and more particularly JAK2 inhibitors are also expected to be useful for the treatment other proliferative diseases including but not limited to autoimmune, inflammatory, neurological, and cardiovascular diseases.

The compounds of Formula (I) should also be useful as standards and reagents in determining the ability of a potential pharmaceutical to inhibit tyrosine kinases, particularly the JAK family and more particularly JAK2. These would be provided in commercial kits comprising a compound of this invention.

### Method 1 (JAK2)

JAK2 kinase activity may be determined by measuring the kinase's ability to phosphorylate synthetic tyrosine residues within a generic polypeptide substrate using an Amplified Luminescent Proximity Assay (Alphascreen) technology (PerkinElmer, 549 Albany Street, Boston, Mass.).

To measure JAK2 kinase activity, a commercially available purified enzyme may be used. The enzyme may be a C-terminal His6-tagged, recombinant, human JAK2, amino acids 808-end, (Genbank Accession number NM 004972) expressed by baculovirus in Sf21 cells (Upstate Biotechnology MA). After incubation of the kinase with a biotinylated substrate and adenosine triphosphate (ATP) for 60 minutes at room temperature, the kinase reaction may be stopped by the addition of 30 mM ethylenediaminetetraacetic acid (EDTA). The reaction may be performed in 384 well microtitre plates and the reaction products may be detected with the addition of streptavidin coated Donor Beads and phosphotyrosine-specific antibodies coated Acceptor Beads using the EnVision Multilabel Plate Reader after an overnight incubation at room temperature.

| | |
|---|---|
| Peptide substrate | TYK2 (Tyr 1054/1055 biotinylated peptide) Cell |
| | Signalling Technology #2200B. 402 μM stock. |
| ATP Km | 30 μM |
| Assay conditions | 150 pM JAK2 enzyme, 5 mM ATP, 80 nM Tyk2, |
| | 10 mM MgCl₂, 50 mM Hepes buffer pH 7.5, 1 |
| | mM DTT, 0.025% Tween20. |
| Incubation | 60 minutes, room temperature |
| Termination/ | 6.3 mM HEPES, 30 mM EDTA, 525 μg/ml BSA, |
| Detection | 40 mM NaCl, 0.007% Triton ® X-100, 12 ng/ml |
| conditions | of Donor Beads, 12 ng/ml of Acceptor Beads |
| Detection incubation | overnight, room temperature |
| Fluometer settings | Excitation = 680 nm Emission = 570 nm |
| | Excitation Time = 180 ms Total Measurement |
| | Time = 550 ms |

Although the pharmacological properties of the compounds of Formula (I) vary with structural change, it is believed that in general, activity possessed by compounds of Formula (I) may be demonstrated at IC₅₀ concentrations (concentrations to achieve 50% inhibition) or doses at a level below 10 μM.

When tested in an in-vitro assay based on the one described for Method 1 (JAK2) above, the JAK inhibitory activity of the following examples were measured at the indicated IC₅₀ values.

| Ex | IC₅₀ (μM) |
|---|---|
| 1 | 0.018 |
| 2 | 0.011 |
| 3 | 0.009 |
| 4 | 0.004 |
| 5 | 0.009 |
| 6 | 0.283 |
| 7 | 3.167 |
| 8 | 0.004 |
| 9 | 0.004 |
| 10 | 0.004 |
| 10(a) | 0.190 |
| 10(b) | <0.008 |
| 14 | 0.007 |
| 15 | 0.873 |
| 16 | 2.874 |
| 17 | 2.875 |
| 18 | 0.013 |
| 19 | 0.003 |
| 20 | 0.007 |
| 21 | 0.004 |
| 22 | 0.004 |
| 23 | 0.086 |
| 26 | 0.219 |
| 28 | 0.798 |
| 29 | 0.004 |
| 30 | <0.003 |
| 31 | 0.234 |
| 32 | 0.393 |
| 33 | 0.998 |
| 34 | 8.319 |
| 35 | 0.023 |

### Method 2 (JAK2)

Alternatively, Janus kinase 2 (JAK2) activity may be determined by measuring the kinase's ability to phosphorylate a tyrosine residue within a peptide substrate using a mobility shift assay on a Caliper LC3000 reader (Caliper, Hopkinton, Mass.), which measures fluorescence of the phosphorylated and unphosphorylated substrate and calculates a ratiometric value to determine percent turnover.

To measure JAK2 kinase activity, an in-house purified enzyme may be used. The enzyme may be a N-terminal GST-tagged, recombinant, human JAK2 (amino acids 831-1132, PLAZA database pAZB0359) expressed in insect cells. After incubation of the kinase with a FAM labeled SRCtide substrate, adenosine triphosphate (ATP), and MgCl₂ for 90 minutes at room temperature, the kinase reaction may be stopped by the addition of 36 mM ethylenediaminetetraacetic acid (EDTA). The reaction may be performed in 384 well microtitre plates and the reaction products may be detected using the Caliper LC3000 Reader.

| | |
|---|---|
| Peptide substrate | SRCtide (5FAM-GEEPLYWSFPAKKK-NH2) |
| | (Anaspec, San Jose, CA) |
| ATP Km | 10 μM |
| Assay conditions | 0.3 nM JAK2 enzyme, 5 mM ATP, 1.5 μM |
| | SRCtide, 10 mM MgCl₂, 50 mM HEPES buffer (pH |
| | 7.3), 1 mM DTT, 0.01% Tween 20, 50 μg/ml BSA |
| Incubation | 90 minutes, room temperature |
| Termination/ | 65 mM HEPES, 36 mM EDTA, 0.2% Coatin Reagent |
| Detection | 3 (Caliper, Hopkinton, MA), 0.003% Tween 20 |
| conditions | |
| Caliper LC3000 | −1.7 PSI, −2000 V downstream voltage, −400 V |
| settings | upstream voltage, 0.2 second sample sip time, 45 |
| | second post sip time, 10% laser strength. |

When tested in an in-vitro assay based on the one described for Method 2 (JAK2) above, the JAK inhibitory activity of the following examples were measured at the indicated IC₅₀ values.

| Ex | IC₅₀ (μM) |
|---|---|
| 11a | 0.986 |
| 11b | 0.021 |
| 24a | 0.073 |
| 24b | 1.71 |
| 25b | >30 |
| 27 | 0.966 |

### Method 3 (JAK2)

Janus kinase 2 (JAK2) activity was determined by measuring the kinase's ability to phosphorylate a tyrosine residue within a peptide substrate using a mobility shift assay on a Caliper LC3000 reader (Caliper, Hopkinton, Mass.), which measures fluorescence of the phosphorylated and unphosphorylated substrate and calculates a ratiometric value to determine percent turnover.

To measure JAK2 kinase activity, an in-house purified enzyme was used. The enzyme was N-terminal GST-tagged, recombinant, human JAK2 (amino acids 831-1132, PLAZA database pAZB0359) expressed in insect cells. After incubation of the kinase with a FAM labeled SRCtide substrate, adenosine triphosphate (ATP), and MgCl₂ for 90 minutes at room temperature, the kinase reaction was stopped by the addition of 36 mM ethylenediaminetetraacetic acid (EDTA). The reaction was performed in 384 well microtitre plates and the reaction products were detected using the Caliper LC3000 Reader.

| | |
|---|---|
| Peptide substrate | SRCtide (5FAM-GEEPLYWSFPAKKK-NH2) |
| | (Anaspec, San Jose, CA) |
| ATP Km | 10 μM |
| Assay conditions | 0.5 nM JAK2 enzyme, 15 μM ATP, 1.5 μM |
| | SRCtide, 10 mM MgCl₂, 50 mM HEPES buffer (pH |
| | 7.3), 1 mM DTT, 0.01% Tween 20, 50 μg/ml BSA |
| Incubation | 90 minutes, room temperature |
| Termination/ | 65 mM HEPES, 36 mM EDTA, 0.2% Coatin Reagent |
| Detection | 3 (Caliper, Hopkinton, MA), 0.003% Tween 20 |
| conditions | |
| Caliper LC3000 | −1.7 PSI, −2000 V downstream voltage, −400 V |
| settings | upstream voltage, 0.2 second sample sip time, 45 |
| | second post sip time, 10% laser strength. |

When tested in an in-vitro assay based on the one described for Method 3 (JAK2) above, the JAK inhibitory activity of the following examples were measured at the indicated IC₅₀ values:

| Ex | IC₅₀ (μM) |
|---|---|
| 12a | 0.138 |
| 12b | <0.003 |
| 13a | 0.180 |
| 13b | <0.003 |

In one aspect, there is provided a compound of Formula (I), or a pharmaceutically acceptable salt thereof, for use as a medicament.

In another aspect, there is provided the use of a compound of Formula (I), or a pharmaceutically acceptable salt thereof, in the manufacture of a medicament for the treatment or prophylaxis of myeloproliferative disorders, myelodysplastic syndrome, and cancer, in a warm-blooded animal such as man.

In still another aspect, there is provided the use of a compound of Formula (I), or a pharmaceutically acceptable salt thereof, in the manufacture of a medicament for the treatment or prophylaxis of myeloproliferative disorders, myelodysplastic syndrome and cancers (solid and hematologic tumors), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acromegaly, acute and chronic inflammation, bone diseases, and ocular diseases with retinal vessel proliferation, in a warm-blooded animal such as man.

In yet another aspect, there is provided the use of a compound of Formula (I), or a pharmaceutically acceptable salt thereof, in the manufacture of a medicament for treating chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and cancers selected from oesophageal cancer, myeloma, hepatocellular, pancreatic, cervical cancer, Ewings sarcoma, neuroblastoma, Kaposi's sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, mesothelioma, renal cancer, lymphoma and leukaemia, in a warm-blooded animal such as man.

In a further aspect, there is provided the use of a compound of Formula (I), or a pharmaceutically acceptable salt thereof, in the manufacture of a medicament for the production of an anti-proliferative effect, in a warm-blooded animal such as man.

In still a further aspect, there is provided the use of a compound of Formula (I), or a pharmaceutically acceptable salt thereof, in the manufacture of a medicament for the production of a JAK inhibitory effect.

In yet a further aspect, there is provided the use of a compound of Formula (I), or a pharmaceutically acceptable salt thereof, in the manufacture of a medicament for the treatment of cancer.

In one aspect, there is provided a method for treating myeloproliferative disorders, myelodysplastic syndrome, and cancer, in a warm-blooded animal such as man, said method comprising administering to said animal an effective amount of a compound of Formula (I), or a pharmaceutically acceptable salt thereof.

In another aspect, there is provided a method for treating myeloproliferative disorders, myelodysplastic syndrome, and cancers (solid and hematologic tumors), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acromegaly, acute and chronic inflammation, bone diseases, and ocular diseases with retinal vessel proliferation, in a warm-blooded animal such as man, said method comprising administering to said animal an effective amount of a compound of Formula (I), or a pharmaceutically acceptable salt thereof.

In still another aspect, there is provided a method for treating chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and cancers selected from oesophageal cancer, myeloma, hepatocellular, pancreatic, cervical cancer, Ewings sarcoma, neuroblastoma, Kaposi's sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, mesothelioma, renal cancer, lymphoma and leukaemia, in a warm-blooded animal such as man, said method comprising administering to said animal an effective amount of compound of Formula (I), or a pharmaceutically acceptable salt thereof.

In yet another aspect, there is provided a method for producing an anti-proliferative effect in a warm-blooded animal such as man, said method comprising administering to said animal an effective amount of a compound of Formula (I), or a pharmaceutically acceptable salt thereof.

In a further aspect, there is provided a method for producing a JAK inhibitory effect in a warm-blooded animal such as man, said method comprising administering to said animal an effective amount of a compound of Formula (I), or a pharmaceutically acceptable salt thereof.

In still a further aspect, there is provided a method for treating cancer in a warm-blooded animal such as man, said method comprising administering to said animal an effective amount of a compound of Formula (I), or a pharmaceutically acceptable salt thereof.

In yet a further aspect, there is provided a compound of Formula (I), or a pharmaceutically acceptable salt thereof, for use in treating myeloproliferative disorders, myelodysplastic syndrome, and cancer, in a warm-blooded animal such as man.

In one aspect, there is provided a compound of Formula (I), or a pharmaceutically acceptable salt thereof, for use in treating myeloproliferative disorders, myelodysplastic syndrome, and cancers (solid and hematologic tumors), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acromegaly, acute and chronic inflammation, bone diseases, and ocular diseases with retinal vessel proliferation, in a warm-blooded animal such as man.

In another aspect, there is provided a compound of Formula (I), or a pharmaceutically acceptable salt thereof, for use in the treating chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and cancers selected from oesophageal cancer, myeloma, hepatocellular, pancreatic, cervical cancer, Ewings sarcoma, neuroblastoma, Kaposi's sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, mesothelioma, renal cancer, lymphoma and leukaemia, in a warm-blooded animal such as man.

In still another aspect, there is provided a compound of Formula (I), or a pharmaceutically acceptable salt thereof, for use in the production of an anti-proliferative effect, in a warm-blooded animal such as man.

In yet another further aspect, there is provided a compound of Formula (I), or a pharmaceutically acceptable salt thereof, for use in the production of a JAK inhibitory effect in a warm-blooded animal such as man.

In a further aspect, there is provided a compound of Formula (I), or a pharmaceutically acceptable salt thereof, for use in the treatment of cancer in a warm-blooded animal such as man.

In still a further aspect, where reference is made to the treatment (or prophylaxis) of cancer, it may particularly refer to the treatment (or prophylaxis) of mesoblastic nephroma, mesothelioma, acute myeloblastic leukemia, acute lymphocytic leukemia, multiple myeloma, oesophageal cancer, myeloma, hepatocellular, pancreatic, cervical cancer, Ewings sarcoma, neuroblastoma, Kaposi's sarcoma, ovarian cancer, breast cancer including secretory breast cancer, colorectal cancer, prostate cancer including hormone refractory prostate cancer, bladder cancer, melanoma, lung cancer—non small cell lung cancer (NSCLC), and small cell lung cancer (SCLC), gastric cancer, head and neck cancer, renal cancer, lymphoma, thyroid cancer including papillary thyroid cancer, mesothelioma, leukaemia, tumors of the central and peripheral nervous system, melanoma, fibrosarcoma including congenital fibrosarcoma and osteosarcoma. More particularly it refers to prostate cancer. In addition, more particularly it refers to SCLC, NSCLC, colorectal cancer, ovarian cancer and/or breast cancer. In a further aspect it may refer to hormone refractory prostate cancer.

In yet a further aspect, there is provided a pharmaceutical composition comprising a compound of Formula (I), or a pharmaceutically acceptable salt thereof, and at least one pharmaceutically acceptable carrier, diluent, or excipient.

In one aspect, there is provided a pharmaceutical composition comprising a compound of Formula (I), or a pharmaceutically acceptable salt thereof, and at least one pharmaceutically acceptable carrier, diluent, or excipient.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients well known in the art. Thus, compositions intended for oral use may contain, for example, one or more coloring, sweetening, flavoring and/or preservative agents.

Suitable pharmaceutically acceptable excipients for a tablet formulation include, for example, inert diluents such as lactose, sodium carbonate, calcium phosphate or calcium carbonate; granulating and disintegrating agents such as corn starch or algenic acid; binding agents such as starch; lubricating agents such as magnesium stearate, stearic acid or talc; preservative agents such as ethyl or propyl p-hydroxybenzoate; and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form or in the form of nano or micronized particles together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives such as ethyl or propyl p-hydroxybenzoate; anti-oxidants such as ascorbic acid); coloring agents; flavoring agents; and/or sweetening agents such as sucrose, saccharine or aspartame.

Oily suspensions may be formulated by suspending the active ingredient in a vegetable oil such as arachis oil, olive oil, sesame oil or coconut oil or in a mineral oil such as liquid paraffin. The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 4 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. Accordingly, the optimum dosage may be determined by the practitioner who is treating any particular patient.

The anti-cancer treatment defined herein may be applied as a sole therapy or may involve, in addition to the compound of the invention, conventional surgery or radiotherapy or chemotherapy. Such chemotherapy may include one or more of the following categories of anti-tumor agents:
(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines including 5-fluorouracil and tegafur, raltitrexed, methotrexate, cytosine arabinoside and hydroxyurea); antitumor antibiotics (for example anthracyclines such as adriamycin, bleomycin, doxorubicin, daunomycin, epirubicin, idarubicin, mitomycin-C, dactinomycin and mithramycin); antimitotic agents (for example vinca alkaloids such as vincristine, vinblastine, vindesine and vinorelbine and taxoids such as taxol and taxotere); and topoisomerase inhibitors (for example epipodophyllotoxins such as etoposide and teniposide, amsacrine, topotecan and camptothecin); and proteosome inhibitors (for example bortezomib [Velcade®]); and the agent anegrilide [Agrylin®]; and the agent alpha-interferon;
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents which inhibit cancer cell invasion (for example metalloproteinase inhibitors such as marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis (2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI-1033)), for example inhibitors of the platelet-derived growth factor family and for example inhibitors of the hepatocyte growth factor family, for example inhibitors or phosphotidylinositol 3-kinase (PI3K) and for example inhibitors of mitogen activated protein kinase (MEK1/2) and for example inhibitors of protein kinase B (PKB/Akt), for example inhibitors of Src tyrosine kinase family and/or Abelson (Abl) tyrosine kinase family such as AZD0530 and dasatinib (BMS-354825) and imatinib mesylate (Gleevec™); and any agents that modify STAT signalling;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(ix) immunotherapy approaches, including for example ex-vivo and in-vivo approaches to increase the immunogenicity of patient tumor cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumor cell lines and approaches using anti-idiotypic antibodies and approaches using the immunomodulatory drugs thalidomide and lenalidomide [Revlimid®]; and
(x) other treatment regimes including: dexamethasone, proteasome inhibitors (including bortezomib), isotretinoin (13-cis retinoic acid), thalidomide, revemid, Rituxamab, ALIMTA, Cephalon's kinase inhibitors CEP-701 and CEP-2563, anti-Trk or anti-NGF monoclonal antibodies, targeted radiation therapy with 1311-metaiodobenzylguanidine (131I-MIBG), anti-G(D2) monoclonal antibody therapy with or without granulocyte-macrophage colony-stimulating factor (GM-CSF) following chemotherapy.

Such conjoint treatment may be achieved by way of the simultaneous, sequential or separate dosing of the individual components of the treatment. Such combination products employ the compounds of this invention, or pharmaceutically acceptable salts thereof, within the dosage range described hereinbefore and the other pharmaceutically-active agent within its approved dosage range.

In addition to its use in therapeutic medicine, compounds of Formula (I) and pharmaceutically acceptable salts thereof are also useful as pharmacological tools in the development and standardization of in vitro and in vivo test systems for the evaluation of the effects of inhibitors of JAK2 in laboratory animals such as cats, dogs, rabbits, monkeys, rats and mice, as part of the search for new therapeutic agents.

In any of the above-mentioned pharmaceutical composition, process, method, use, medicament, and manufacturing features of the instant invention, any of the alternate embodiments of the compounds of the invention described herein also apply.

In one aspect, the inhibition of JAK activity particularly refers to the inhibition of JAK1 activity.

In another aspect, the inhibition of JAK activity particularly refers to the inhibition of JAK2 activity.

### Process

It is noted that many of the starting materials for synthetic methods as described herein are commercially available and/or widely reported in the scientific literature, or could be made from commercially available compounds using adaptations of processes reported in the scientific literature. The skilled chemist is further referred to Advanced Organic Chemistry, 5^{th} Edition, by Jerry March and Michael Smith, published by John Wiley & Sons 2001, for general guidance on reaction conditions and reagents.

If not commercially available, the necessary starting materials for the procedures such as those described herein may be made by procedures which are selected from standard organic chemical techniques, techniques which are analogous to the synthesis of known, structurally similar compounds, or techniques which are analogous to the described procedure or the procedures described in the Examples. The skilled chemist will be able to use and adapt the information contained and referenced within the above references, and accompanying examples therein and also the Examples, Procedures, and Scheme herein, to obtain necessary starting materials and products.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, published by John Wiley and Sons, 1991) and as described hereinabove.

Compounds of Formula (I) may be prepared in a variety of ways. The Schemes and Processes shown below illustrate some methods for synthesizing compounds of Formula (I) and intermediates which may be used for the synthesis of compounds of Formula (I) (wherein Ring A, Ring B, Ring C, R², R³, R⁴, m, and n, unless otherwise defined, are as defined hereinabove). Where a particular solvent or reagent is shown in a Scheme or Process, or referred to in the accompanying text, it is to be understood that the chemist of ordinary skill in the art will be able to modify that solvent or reagent as necessary. The Schemes and Processes are not intended to present an exhaustive list of methods for preparing the compounds of Formula (I); rather, additional techniques of which the skilled chemist is aware may be also be used for the compounds' synthesis. The claims are not intended to be limited to the structures shown in the Processes and Scheme.

In one aspect, compounds of Formula (I) may be prepared by:

1) Process A—reacting a compound of Formula (A):

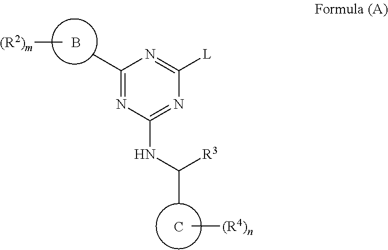

with a compound of Formula (B):

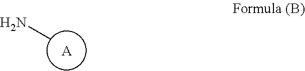

2) Process B—reacting a compound of Formula (C)

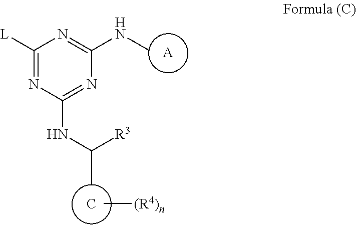

with a compound of Formula (D)

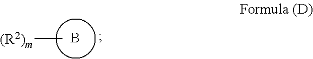

3) Process C—reacting a compound of Formula (E)

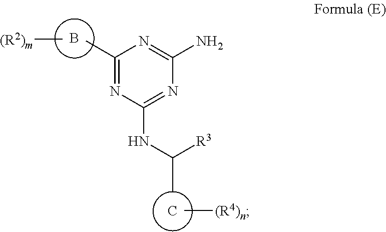

with a compound of Formula (F)

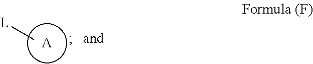

4) Process D—reacting a compound of Formula (G)

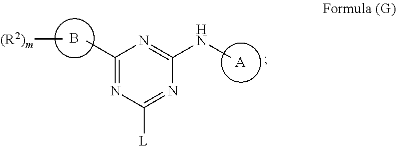

with a compound of Formula (H)

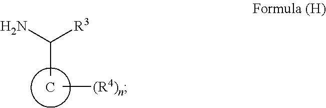

and thereafter if appropriate:
i) converting a compound of Formula (I) into another compound of Formula (I);
ii) removing any protecting groups; and/or
iii) forming a pharmaceutically acceptable salt,
   wherein L in each occurrence may be the same or different, and is a leaving group, as discussed hereinabove.

More particularly, with regard to Process A, the compound of Formula (A) and the compound of Formula (B) may be reacted together in the presence of a suitable solvent, examples of which include ketones such as acetone, alcohols such as ethanol and butanol, and aromatic hydrocarbons such as toluene and N-methyl pyrrolid-2-one. Such reaction may advantageously occur in the presence of a suitable base, examples of which include inorganic bases such as potassium carbonate and cesium carbonate organic bases such as triethylamine and diisopropylethyl amine. The reaction is advantageously performed at a temperature in a range from 0° C. to reflux.

In another aspect, the compound of Formula (A) and the compound of Formula (B) may be reacted together under standard Buchwald conditions (for example see J. Am. Chem. Soc., 118, 7215; J. Am. Chem. Soc., 119, 8451; J. Org. Chem., 62, 1568 and 6066), with a suitable base. Examples of suitable bases include inorganic bases such as cesium carbonate, and organic bases such as potassium t-butoxide. Such a reaction may be advantageously occur in the presence of palladium acetate. Solvents suitable for such a reaction include aromatic solvents such as toluene, benzene, or xylene.

Each of Processes B, C, and D may be performed under the conditions described for the reaction of the compound of Formula (A) with the compound of Formula (B) in Process A.

In one aspect, compounds of Formula (L) (which are compounds of Formula (H) having the indicated stereochemistry) may be prepared via chiral synthesis according to Scheme 1.

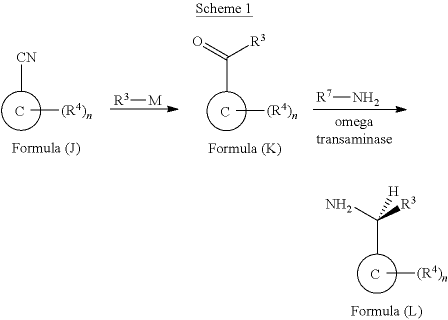

Reaction of a compound of Formula (J) with an organometallic reagent R⁴-M (in which R⁴ is an alkyl group such as methyl, and M is a metal species such as —MgCl, —MgBr or —Li), followed by quenching, may be used to obtain a compound of Formula (H). Reaction of a compound of Formula (K) with amine donor R⁷—NH₂ (in which R⁷ is a group such as isopropyl or methylbenzyl) in the presence of an omega transaminase may be used to obtain a compound of Formula (L). Suitable amine donors may include alanine in the presence of pyruvatedecarboxylase, benzylamine, S-methylbenzylamine and isopropylamine. Suitable omega transaminases include those from Vibrio fluvalis, thermostable transaminase CNB05-01, Biocatalytics® 101, 102, 103, 110, 111, 114, 115. The biocatalysts maybe free enzymes or suitable whole cell preparations. Before reaction with the compound of Formula (K), the omega transaminase and R⁷—NH₂ may advantageously be mixed in solution with an aqueous buffer such as aqueous potassium phosphate or aqueous HEPES buffer, followed by addition of pyridoxyl phosphate. In the case of an immiscible organic solvent (such as toluene, BuOAc or diisooctylphthalate) may or may not be advantageously added. The stereoselectivity of the amine can be switched from S to R by using an R selective transaminase such as Biocatalytics® 117.

### EXAMPLES

The invention will now be further described with reference to the following illustrative Examples in which, unless stated otherwise:
(i) temperatures are given in degrees Celsius (° C.); operations are carried out at room temperature or ambient temperature, that is, in a range of 18-25° C.;
(ii) organic solutions were dried over anhydrous magnesium sulfate unless other wise stated; evaporation of organic solvent was carried out using a rotary evaporator under reduced pressure (4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC or liquid chromatography/mass spectroscopy and reaction times are given for illustration only;
(v) final products have satisfactory proton nuclear magnetic resonance (NMR) spectra and/or mass spectra data;
(vi) yields are given for illustration only and are not necessarily those which can be obtained by diligent process development; preparations were repeated if more material was required;
(vii) when given, NMR data is in the form of delta values for major diagnostic protons, given in part per million (ppm) relative to tetramethylsilane (TMS) as an internal standard, determined at 300 MHz in DMSO-d₆ unless otherwise stated;
(viii) chemical symbols have their usual meanings;
(ix) solvent ratio is given in volume:volume (v/v) terms.
(x) “ISCO” refers to normal phase flash column chromatography using pre-packed silica gel cartridges (12 g, 40 g etc.), used according to the manufacturer's instructions, obtained from Teledyne ISCO, Inc, 4700 Superior Street Lincoln, Nebr., USA.
(xi) A “Gilson® column” refers to a YMC-AQC18 reverse phase HPLC Column with dimension 20 mm/100 and 50 mm/250 in H₂O/MeCN with 0.1% TFA as mobile phase unless otherwise stated and used according to the manufacturer's instructions, obtained from Gilson®, Inc. 3000 Parmenter Street, Middleton, Wis. 53562-0027, U.S.A.
(xii) “SFC (super critical fluid chromatography)” refers to Analytical SFC (ASC-1000 Analytical SFC System with Diode Array Detector) and/or Preparative SFC (APS-1000 AutoPrep Preparative SFC), used according to the manufacturer's instruction, obtained from SFC Mettler Toledo AutoChem, Inc. 7075 Samuel Morse Drive Columbia Md. 21046, U.S.A.
(xiii) Parr Hydrogenator or Parr shaker type hydrogenators are systems for treating chemicals with hydrogen in the presence of a catalyst at pressures up to 5 atmospheres (60 psi) and temperatures to 80° C.
(xiv) the following abbreviations have been used:
   atm atmosphere
   BINAP 2,2′-bis(diphenylphosphino)-1,1′-binapthyl
   Boc₂O di-tert-butyl-dicarbonate
   DCM dichloromethane
   DIPEA N,N-diisopropylethylamine
   DMF N,N-dimethylformamide
   DMAP 4-dimethylaminopyridine
   DMSO dimethylsulfoxide
   dppf 1,1′-Bis(diphenylphosphino)ferrocene
   EtOAc ethyl acetate
   Et₂O diethyl ether
   GC gas chromatography
   HPLC high-performance liquid chromatography
   LDA lithium diisopropylamide
   LCMS liquid chromatography/mass spectroscopy
   MTBE methyl t-butyl ether
   Pd₂(dba)₃ tris(dibenzylideneacetone)dipalladium (0)
   SEM 2-(trimethylsilyl)ethoxy)methyl
   THF tetrahydrofuran
   TFA trifluoroacetic acid
   TEA triethylamine
   e.e. enantiomeric excess
   Xantphos® 4,5-bis(diphenylphosphino)-9,9-dimethylxanthene

The Examples are illustrative and are not to be read as limiting the scope of the invention as defined by the claims.

### Intermediate 1

### 1-Methyl-4-nitro-1H-imidazole

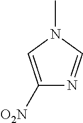

4-Nitro-1H-imidazole (2 g, 17.69 mmol) was dissolved in acetonitrile (20 mL), and potassium carbonate (3.67 g, 26.53 mmol) and iodomethane (1.327 mL, 21.22 mmol) were added. The reaction mixture was then heated at 65° C. overnight. The reaction mixture was filtered and the filtrate was concentrated in vacuo leaving a reddish orange solid (3.214 g). This material was purified by ISCO (0-10% MeOH/DCM). Concentration of the fractions in vacuo provided the title product as a yellow solid (2.058 g).

LCMS: 128 [M+H]⁺.

### Intermediate 2

### 4,6-Dichloro-N-(1-methyl-1H-imidazol-4-yl)-1,3,5-triazin-2-amine

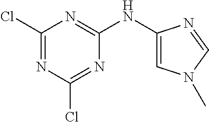

1-Methyl-4-nitro-1H-imidazole (Intermediate 1, 500 mg, 3.93 mmol) was dissolved in ethanol (7.868 mL) and Pd/C (10 wt. %, Degussa®, 105 mg, 0.10 mmol) was added. The reaction mixture was subjected to 1 atm of hydrogen for 3 hours. The reaction mixture was filtered and the filtrate was cooled to 0° C. 2,4,6-trichloro-1,3,5-triazine (580 mg, 3.15 mmol) and TEA (1.097 mL, 7.87 mmol) were then added. The reaction mixture was allowed to warm to 25° C. overnight. The reaction mixture was then filtered providing the title product as a tan solid (572 mg).

LCMS: 246 [M+H]⁺.

### Intermediate 3

### 1-(3,5-Difluoropyridin-2-yl)-2-methoxyethanone

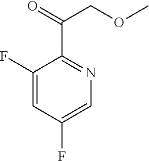

3,5-Difluoropyridine (5.0 g, 43.45 mmol) in THF was cooled to −72° C. (external −80° C.). LDA (23.9 mL, 1.1 eq.) was added drop-wise so that the internal temperature did not increase more than 3° C. during addition. The reaction mixture turned into a deep brownish, thick phase. The reaction mixture was stirred for 30 mins. TMS-Cl (43.4 mL, 43.45 mmol) was added in a relatively fast fashion. The reaction became a clear and light yellow solution. LDA (23.9 mL, 1.1 eq.) was added drop-wise in a quicker version, and the reaction mixture was allowed to stir for 2 hours. Methyl 2-methoxyacetate (5.59 mL, 56.48 mmol) was added quickly through a syringe. The reaction mixture was quenched at −78° C. by adding 20 ml of saturated NH₄Cl solution. Evaporation of the organic extracts under reduced pressure gave a colored residue. Purification by ISCO (0-25% EtOAc/hexanes), gave the title product (3 g).

LCMS: 188 [M+H]⁺.

### Intermediate 4

### 1-(3,5-Difluoropyridin-2-yl)-N-hydroxy-2-methoxyethanimine

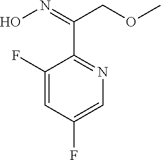

1-(3,5-Difluoropyridin-2-yl)-2-methoxyethanone (Intermediate 3) was dissolved in ethanol (255 ml, 10 vol). Hydroxylamine hydrochloride (14.22 g, 204.61 mmol) was added, followed by drop-wise addition of TEA (28.5 ml, 204.61 mmol). The resulting colored mixture was heated to 50° C. for 2 hours. The volatiles were evaporated under reduced pressure and the residue was partitioned between water (255 ml) and ethyl acetate (255 ml). The separated aqueous layer was further extracted into 2× ethyl acetate (255 ml). The combined organic extracts washed with water (255 ml), saturated brine (255 ml), dried over MgSO₄, filtered and concentrated in vacuo to give 42 g of a brown oil. Purification by column chromatography (25-40% EtOAc in isohexanes) gave 32 g of the title product as yellow oily solid (˜3:1 mixture of isomers). Trituration in MTBE gave the title product (12.3 g, 60.84 mmol, 44.6%, single isomer) as a white solid. The liquor was evaporated under reduced pressure and the residue was re-columned using the previous conditions followed by trituration with EtOAc/isohexanes to give additional 1-(3,5-difluoropyridin-2-yl)-2-methoxyethanone oxime (7.2 g, 35.62 mmol, 26.1%).

LCMS: 203 [M+H]⁺.

### Intermediate 5

### (1R)-1-(3,5-Difluoropyridin-2-yl)-2-methoxyethanamine, (R)-mandelic acid salt

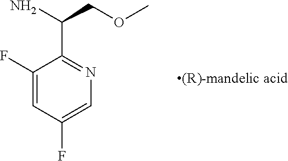

1-(3,5-Difluoropyridin-2-yl)-N-hydroxy-2-methoxyethanimine (Intermediate 4) was dissolved in EtOAc (0.4M) and was subsequently subjected to catalytic hydrogenation (Pd on C) in a Parr Hydrogenator (Pressure 5 bar at 40° C.) for 1 hour. The catalyst was filtered through diatomaceous earth (Celite®) and the filtrate of 1-(3,5-difluoropyridin-2-yl)-2-methoxyethanamine (0.4 M in ethyl acetate, 180 mL, 72.00 mmol) was treated with (R)-Mandelic acid (5.81 g, 38.16 mmol). Precipitation was observed almost instantaneously and the resulting mixture was allowed to stir overnight. (R)-1-(3,5-difluoropyridin-2-yl)-2-methoxyethanamine (R)-mandelate salt was collected via filtration (8.5 g, 69.4%). The other enantiomer, (S)-1-(3,5-difluoropyridin-2-yl)-2-methoxyethanamine, (R)-mandelic acid salt was recovered after evaporation of the mother liquor.

¹H NMR (400 MHz) δ ppm 8.6 (s, 1H), 8.01 (m, 1H), 7.41 (t, 2H), 7.36 (t, 2H), 7.19 (m, 1H), 4.81 (s, 1H), 4.50 (m, 1H), 3.57 (d, 2H), 3.23 (s, 3H).

LCMS: 188 [M−H]⁺.

### Intermediate 6

### 6-Chloro-N-[(1R)-1-(3,5-difluoropyridin-2-yl)-2-methoxyethyl]-N′-(1-methyl-1H-imidazol-4-yl)-1,3,5-triazine-2,4-diamine

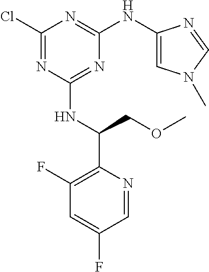

(1R)-1-(3,5-Difluoropyridin-2-yl)-2-methoxyethanamine, (R)-mandelic acid salt (Intermediate 5, 874 mg, 2.57 mmol) was dissolved in ethanol (8 mL), and TEA (1.301 mL, 9.34 mmol) and 4,6-dichloro-N-(1-methyl-1H-imidazol-4-yl)-1,3,5-triazin-2-amine (Intermediate 2, 572 mg, 2.33 mmol) were added. The reaction mixture was stirred overnight at 25° C. The reaction mixture was filtered and an off-white solid (698 mg) was collected. This material was purified by ISCO (2-10% MeOH/DCM). Concentration of the fractions in vacuo provided the title product as a white solid (554 mg).

LCMS: 397 [M+H]⁺.

### Intermediate 7

### 5-Fluoropyrimidine-2-carbonitrile

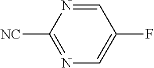

A 10 ml microwave vial was charged with 2-chloro-5-fluoropyrimidine (2.0 g, 15.09 mmol), Pd₂(dba)₃ (0.549 g, 0.6 mmol), dppf (0.67 g, 1.21 mmol), zinc cyanide (1.15 g, 9.81 mmol), and zinc dust (0.237 mg, 3.62 mmol). The flask was evacuated and backfilled with N₂ and anhydrous dimethylacetamide. The vial was mounted onto a Personal Chemistry microwave reactor and heated at 100° C. for 10 hours. The reaction mixture was diluted with EtOAc and then washed with brine three times. The layers were separated, and the organic layer was evaporated to dryness. The dried residue was purified by silica gel chromatography (By ISCO Combiflash with gradient EtOAc and hexanes) to afford the title product as a creamy solid (1.50 g, 80%). ¹H NMR (CDCl₃) δ: 8.80 (s, 2H).

GC-MS: 123 [M].

### Intermediate 8

### N-[1-(5-Fluoropyrimidin-2-yl)ethenyl]acetamide

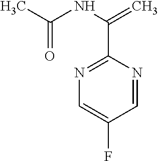

5-Fluoropyrimidine-2-carbonitrile (Intermediate 7, 1.0 g, 8.1 mmol) in THF (10 ml) was added to a solution of MeMgBr (3.3 ml, 9.75 mmol) in ether drop wise at 0° C. After addition, the reaction mixture was warmed to room temperature, stirred at room temperature for 1 hour, and then diluted with DCM (10 ml). Acetic anhydride (1.23 ml, 13.0 mmol) was added in one portion. The reaction mixture was stirred at room temperature for 1 hour and 40° C. for 1 hour. Saturated sodium bicarbonate solution (10 ml) was added and extracted with EtOAc (2×20 ml). The combined organic phases were dried over sodium sulfate. After removal of solvent, the resulting residue was purified by column chromatography (2.5:1 v/v hexane:EtOAc) to give the title product as a white solid (0.38 g, 26%).

¹H NMR (400 MHz) δ: 9.34 (s, 1H), 8.95 (s, 2H), 6.25 (s, 1H), 6.03 (s, 1H), 2.11 (s, 3H).

LCMS: 182 [M+H]⁺.

### Intermediate 9

### Method A

### N-[(1S)-1-(5-Fluoropyrimidin-2-yl)ethyl]acetamide

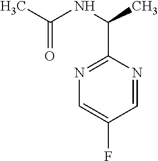

To a solution of N-[1-(5-fluoropyrimidin-2-yl)ethenyl]acetamide (Intermediate 8, 0.10 g, 0.55 mmol) in MeOH (5 ml) under N₂ was added (+)-1,2-bis((2S,5S)-2,5-diethylphospholano)benzene (cyclooctadiene)rhodium(I)trifluoromethanesulfonate (0.04 g, 0.0055 mmol). The solution was transferred to a high pressure bomb and charged with 150 psi H₂. The reaction mixture was stirred at room temperature for 4 hours. The solvent was removed and the resulting residue was purified by column chromatography (EtOAc) to give the title product as a white solid (0.096 g, 95%).

¹H NMR (400 MHz) δ: 8.84 (d, 2H), 8.34 (d, 1H), 5.00 (m, 1H), 1.84 (s, 3H), 1.37 (d, 3H).

LCMS: 184 [M+H]⁺.

Enantiomeric excess determined by HPLC (Chiralpak® IA; 95:5 CO₂/MeOH), >99% ee.

### Intermediate 9

### Method B

### N-[(1S)-1-(5-Fluoropyrimidin-2-yl)ethyl]acetamide

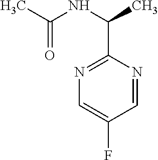

A solution of MeMgCl (268 ml, 0.81 mol) in tetrahydrofuran was added to a solution of 5-fluoropyrimidine-2-carbonitrile (Intermediate 7, 82.5 g, 0.65 mol) in 2-methyltetrahydrofuran (600 ml) at −40° C. On complete reaction, the reaction mixture was warmed to −25° C. and transferred into a solution of aqueous hydrochloric acid (475 ml, 1.98 mol). On complete reaction, the phases were separated and the aqueous phase extracted with further 2-methyltetrahydrofuran. The organic phases were combined and concentrated by evaporation before adding heptane to crystallize the product as a light brown crystalline solid (73.2 g, 80%).

¹H NMR (400 MHz) δ: 9.08 (d, 2H), 2.68 (s, 3H).

LCMS: 141 [M+H]⁺.

(S)-Methylbenzylamine (24.2 ml, 0.19 mol) was added to a solution of monobasic potassium phosphate (4.7 g, 0.34 mol) in water (360 ml). The pH of the solution was adjusted to pH 7.5 by the addition of acetic acid. Pyridoxal phosphate (0.23 g, 0.85 mmol) was added, followed by 2-acetyl-5-fluoropyrimidine (24.0 g, 0.17 mol), a buffered solution of an omega transaminase (from Vibrio fluvalis, 48 ml, 9.3KU) and toluene (120 ml). The reaction mixture was adjusted to pH7.5 with potassium carbonate then held at 29° C. for 18 hours. The reaction mixture was filtered and the organic layer discarded. Potassium carbonate (45.4 g, 0.33 mol) was added to the aqueous phase followed by a solution of di-tert-butyl dicarbonate (40.9 g, 0.19 mol) in 2-methyltetrahydrofuran (192 ml). The mixture was filtered and the aqueous layer extracted with further 2-methyltetrahydrofuran. The organic layers were combined and evaporated to dryness. The residue was dissolved in MTBE (96 ml) and a solution of 5-6N hydrochloric acid in isopropanol (78 ml, 0.43 mol) was added. The reaction mixture was heated to 40° C. to precipitate the product, which was isolated as a crystalline solid (24.3 g, 79%).

¹H NMR (400 MHz) δ: 9.02 (d, 2H), 4.55 (m, 1H), 1.58 (d, 3H).

LCMS: 142 [M+H]⁺.

Enantiomeric excess was determined by chiral HPLC (CrownPak CR+, aqueous perchloric acid, >99% ee S-enantiomer).

### Intermediate 10

### tert-Butyl [(1S)-1-(5-fluoropyrimidin-2-yl)ethyl]carbamate

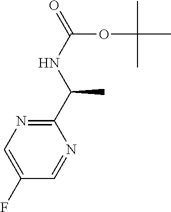

N-[(1S)-1-(5-Fluoropyrimidin-2-yl)ethyl]acetamide (Intermediate 9, 0.20 g, 1.09 mmol), DMAP (0.027 g, 0.22 mmol) and Boc₂O (0.60 g, 2.73 mmol) in THF (10 ml) were stirred at 50° C. for 40 hours. After cooling to room temperature, lithium hydroxide monohydrate (0.094 g, 2.24 mmol) and water (10 ml) was added. The reaction mixture was stirred at room temperature for 9 hours. Ether (30 ml) was added, the organic layer was separated, washed with brine (20 ml), and dried over sodium sulfate. After removal of solvent, the resulting residue was purified by column chromatography (Hex-EtOAc=5:1) to give the title product as a pale yellow oil (0.21 g, 80%).

¹H NMR (400 MHz) δ: 8.84 (s, 2H), 7.24 (d, 1H), 4.74 (m, 1H), 1.35 (s, 12H).

LCMS: 242 [M+H]⁺.

### Intermediate 11

### (1S)-1-(5-Fluoropyrimidin-2-yl)ethanamine hydrochloride

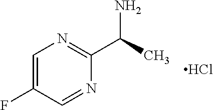

To a solution of tert-butyl [(1S)-1-(5-fluoropyrimidin-2-yl)ethyl]carbamate (Intermediate 10, 0.21 g, 0.87 mmol) in DCM (5 ml) was added HCl (1.3 ml, 5.2 mmol) in dioxane. The reaction mixture was stirred at room temperature for 3 hours. The solvent was removed to give the title product as white solid (quantitative).

LCMS: 142 [M+H]⁺.

### Intermediate 12

### 6-Chloro-N-[(1S)-1-(5-fluoropyrimidin-2-yl)ethyl]-N′-(1-methyl-1H-imidazol-4-yl)-1,3,5-triazine-2,4-diamine

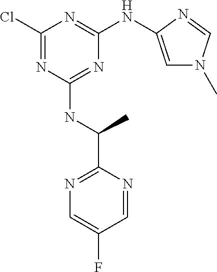

(1S)-1-(5-Fluoropyrimidin-2-yl)ethanamine hydrochloride (Intermediate 11, 77 mg, 0.43 mmol) in EtOH (5 mL), at 0° C. was treated with triethylamine (0.151 mL, 1.08 mmol). The resulting mixture was stirred for 10 minutes whereupon 4,6-dichloro-N-(1-methyl-1H-imidazol-4-yl)-1,3,5-triazin-2-amine (Intermediate 2, 106 mg, 0.43 mmol) was added in one portion. The resulting solution was allowed to warm up overnight to room temperature. The volatiles were evaporated under reduced pressure to give an oil. Purification by ISCO provided the title product (150 mg).

### Intermediate 13

### 4-Nitro-1-(2-phenylethyl)-1H-imidazole

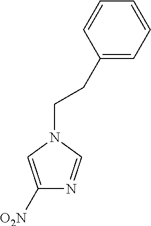

4-Nitro-1H-imidazole (3 g, 26.53 mmol) and (2-bromoethyl)benzene (5.46 mL, 39.80 mmol) were reacted using a procedure similar to the one described for the synthesis of Intermediate 1, providing the title product (0.86 mg).

LCMS: 218 [M+H]⁺.

### Intermediate 14

### 4,6-Dichloro-N-[1-(2-phenylethyl)-1H-imidazol-4-yl]-1,3,5-triazin-2-amine

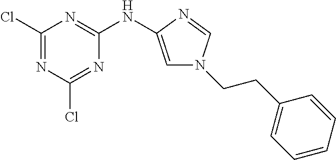

4-Nitro-1-(2-phenylethyl)-1H-imidazole (Intermediate 13, 0.86 g, 3.96 mmol), Fe metal (1.105 g, 19.80 mmol) and ammonium chloride (0.424 g, 7.92 mmol) were loaded in a round-bottom flask followed by the addition of MeOH (10 mL) and water (10.00 mL). The resulting solution was heated to 80° C. for 1 hour whereupon it was filtered, and the filtrate was evaporated under reduced pressure. The residue was dissolved in acetone, and the precipitate was removed by filtration and evaporation under reduced pressure, giving an oil. This oil was re-dissolved in ethanol (10.00 mL) cooled to 0° C. 2,4,6-trichloro-1,3,5-triazine (580 mg, 3.15 mmol) and TEA (1.097 mL, 7.87 mmol) were then added and the reaction mixture was allowed to warm to 25° C. overnight. The reaction mixture was then filtered, providing the title product (250 mg).

LCMS: 336 [M+H]⁺.

### Intermediate 15

### 6-Chloro-N-[(1S)-1-(5-fluoropyrimidin-2-yl)ethyl]-N′-[1-(2-phenylethyl)-1H-imidazol-4-yl]-1,3,5-triazine-2,4-diamine

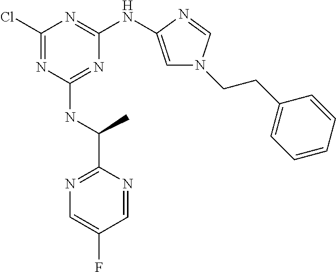

4,6-Dichloro-N-[1-(2-phenylethyl)-1H-imidazol-4-yl]-1,3,5-triazin-2-amine (Intermediate 14, 220 mg, 0.66 mmol) and (1S)-1-(5-fluoropyrimidin-2-yl)ethanamine hydrochloride (Intermediate 11, 117 mg, 0.66 mmol), were reacted using a procedure similar to the one described for the synthesis of Intermediate 12, providing the title product (350 mg).

### Intermediate 16

### 2-Chloro-1,3-thiazole-5-carbonitrile

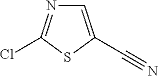

A dried flask under nitrogen was charged with acetonitrile (7.990 mL), and copper(II) chloride (645 mg, 4.79 mmol) was added. The reaction mixture was maintained in a 25° C. bath, and tert-Butyl nitrite (0.712 mL, 5.99 mmol) was added over 10 minutes. After an additional 10 minutes, 2-aminothiazole-5-carbonitrile (500 mg, 4.00 mmol) was added gradually and the reaction mixture was stirred at 25° C. for 5 hours. 0.5M HCl (20 mL) was added to the reaction mixture and the organics were extracted with EtOAc, washed with brine, and dried over Na₂SO₄. Concentration in vacuo gave a rust colored oil that slowly began to crystallize in the flask. This material was purified by ISCO (100% DCM isocratic). Concentration of the fractions in vacuo provided the title product as a yellow crystalline solid (372 mg).

¹H NMR (300 MHz, CHLOROFORM-d) δ ppm 8.07 (s, 1H).

### Intermediate 17

### 6-Chloro-N-[(1S)-1-(5-fluoropyrimidin-2-yl)ethyl]-1,3,5-triazine-2,4-diamine

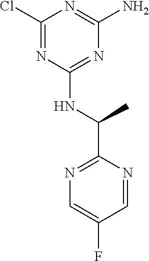

To a solution of 4,6-dichloro-1,3,5-triazin-2-amine (1 g, 6.06 mmol) in acetonitrile (17.32 ml) was added (1S)-1-(5-fluoropyrimidin-2-yl)ethanamine hydrochloride (Intermediate 11, 1.077 g, 6.06 mmol), followed by DIPEA (2.117 ml, 12.12 mmol) at 25° C. The mixture was stirred overnight at room temperature, whereupon it was diluted with EtOAc. The organic phase was washed with brine, H₂O and dried. Evaporation of the volatiles under reduced pressure the title product (1.6 g) as white solid.

LCMS: 270 [M+H]⁺.

### Intermediate 18

### N-[(1S)-1-(5-Fluoropyrimidin-2-yl)ethyl]-6-morpholin-4-yl-1,3,5-triazine-2,4-diamine

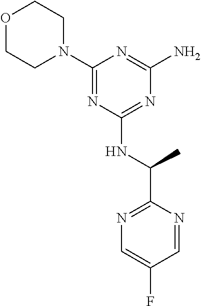

To a solution of 6-chloro-N-[(1S)-1-(5-fluoropyrimidin-2-yl)ethyl]-1,3,5-triazine-2,4-diamine (Intermediate 17, 0.817 g, 3.03 mmol) in acetonitrile (6.06 ml) was added morpholine (0.792 ml, 9.09 mmol) followed by DIPEA (0.529 ml, 3.03 mmol). The resulting mixture was allowed to stir at ambient temperature for 12 hours. Evaporation of the volatiles under reduced pressure gave a yellow oil. Purification by column chromatography (ISCO, 0%→10% MeOH/DCM) afforded the title product (675 mg).

LCMS: 321 [M+H]⁺.

### Intermediate 19

### 4-Chloro-N-[(1S)-1-(5-fluoropyrimidin-2-yl)ethyl]-6-morpholin-4-yl-1,3,5-triazin-2-amine

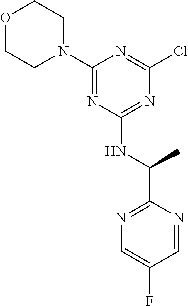

2,4,6-Trichloro-1,3,5-triazine (3.69 g, 20 mmol) in ethanol (80 ml) was cooled to −78° C. In a separate flask, (1S)-1-(5-fluoropyrimidin-2-yl)ethanamine hydrochloride (Intermediate 11, 3.55 g, 20.00 mmol) in ethanol (20 ml) was treated with DIPEA (6.99 ml, 40.00 mmol) and the resulting mixture was stirred for 30 minutes whereupon it was added drop-wise to a flask containing 2,4,6-trichloro-1,3,5-triazine (3.69 g, 20 mmol) in ethanol (80 ml) pre-cooled to −78° C. The reaction mixture was stirred at −78° C. for 2 hours. The reaction mixture was re-cooled to −78° C., morpholine (1.742 ml, 20.00 mmol) and DIPEA (3.49 ml, 20.00 mmol) in ethanol (10 ml) were added drop-wise via syringe. The reaction mixture was stirred at −78° C. for 2 h and subsequently at room temperature overnight. The volatiles were removed under reduced pressure and the residue was partitioned between CH₂Cl₂ and H₂O. The organic phase was dried and concentrated in vacuo to yield the title product.

LCMS: 340 [M+H]⁺.

### Intermediate 20

### 1-(3,5-Difluoropyridin-2-yl)ethanone

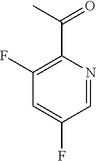

A solution of methylmagnesium bromide (36.8 ml, 117.78 mmol) in THF (50 ml) was stirred under N₂ and cooled to −78° C. 3,5-Difluoropicolinonitrile (15.0 g, 107.07 mmol) in THF (50 ml) was added drop wise with an addition funnel at such a rate that the internal temperature was kept below −4° C. After the addition was complete, the reaction mixture was poured into a 1M HCl (100 ml, chilled in an ice bath). The reaction mixture was stirred at 0° C. for 30 minutes and room temperature for 30 minutes. To this solution 150 ml of EtOAc was added to extract product. The aqueous phase was neutralized to pH 9 with NaHCO₃ and extracted with EtOAc (2×20 ml). The organic layers were combined and the volatiles were removed under reduced pressure. Purification by ISCO (0-10% EtOAc-hexanes) gave the title product as light yellow oil.

LCMS: 158 [M+H]⁺.

### Intermediate 21

### 1-(3,5-Difluoropyridin-2-yl)-N-hydroxyethanimine

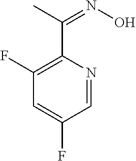

To a solution of 1-(3,5-difluoropyridin-2-yl)ethanone (Intermediate 20, 12.91 g, 82.17 mmol) in ethanol (164 ml) was added hydroxylamine hydrochloride (8.56 g, 123.25 mmol) followed by Et₃N (17.18 ml, 123.25 mmol) and the resulting mixture was stirred overnight at room temperature. The volatiles were removed under reduced pressure and the residue was partitioned between EtOAc/H₂O. The organic extracts were washed with brine and dried. An orange yellow solid was obtained, and purification by ISCO (10% EtOAc/hexanes→25% EtOAc/hexanes) gave the title product (9.73 g, 68.8%) as a yellow solid.

¹H NMR (300 MHz, DMSO-d₆) δ ppm 2.19 (s, 3H), 7.98 (ddd, J=10.97, 8.81, 2.26 Hz, 1H), 8.55 (d, J=2.26 Hz, 1H), 11.70 (s, 1H).

LCMS: 173 [M+H]⁺.

### Intermediate 22

### (1S)-1-(3,5-Difluoropyridin-2-yl)ethanamine, (R)-mandelic acid salt

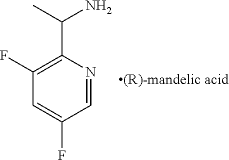

1-(3,5-Difluoropyridin-2-yl)-N-hydroxyethanimine (Intermediate 21, 9.73 g, 56.53 mmol) was added to water (113 ml) to form a suspension. Ammonium hydroxide (22.01 ml, 565.26 mmol) was added to the above solution, followed by ammonium acetate (5.23 g, 67.83 mmol). The mixture was heated at 50° C. and subsequently zinc (14.79 g, 226.11 mmol) was added portion wise, while maintaining the internal temperature below 65° C. After the addition was complete, the reaction mixture was stirred at 50° C. for 3 hours. Solid NaCl and EtOAc were added to quench the reaction. The reaction mixture was stirred for 1 hour at room temperature, was then filtered through diatomaceous earth (Celite®), and rinsed with EtOAc. The organic layer was washed with 5 ml 2.5% NaOH (aq.), followed by 10 ml NH₄OH. The organic layer was then washed with brine and dried with Na₂SO₄. The organic layer was concentrated under reduced pressure to obtain the title product as light yellow oil.

¹H NMR (400 MHz, MeOD) δ ppm 1.62 (d, J=6.82 Hz, 3H), 4.86 (q, J=6.82 Hz, 1H), 7.75 (ddd, J=10.11, 8.34, 2.27 Hz, 1H), 8.49 (d, J=2.27 Hz, 1H).

1-(3,5-Difluoropyridin-2-yl)ethanamine (0.83 g, 5.25 mmol) and (R)-mandelic acid (0.399 g, 2.62 mmol) in ethyl acetate (10 mL) were heated to 50° C. A solid formed after heating for a few minutes. Stirring was continued for 1 hour at 50° C. The reaction mixture was then cooled to ambient temperature. The solid was collected via gravity filtration (no vacuum) washing with ethyl acetate until the orange color disappeared. The solid (265 mg) was identified as the title product (e.e >98%).

### Intermediate 23

### 6-Chloro-N-[(1S)-1-(3,5-difluoropyridin-2-yl)ethyl]-N′-(1-methyl-1H-imidazol-4-yl)-1,3,5-triazine-2,4-diamine

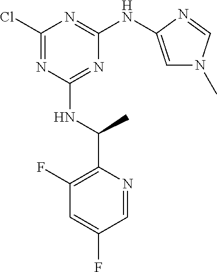

(1S)-1-(3,5-Difluoropyridin-2-yl)ethanamine, (R)-mandelic acid salt (Intermediate 22, 627 mg, 2.02 mmol) was dissolved in ethanol (8 mL) and TEA (1.024 mL, 7.34 mmol) and 4,6-dichloro-N-(1-methyl-1H-imidazol-4-yl)-1,3,5-triazin-2-amine (Intermediate 2, 450 mg, 1.84 mmol) were added. The reaction mixture was then stirred overnight at 25° C. The reaction mixture was then filtered providing the title product as an off-white solid (527 mg).

LCMS: 367 [M+H]⁺.

### Intermediate 24

### 1-(3,5-Difluoropyridin-2-yl)ethanamine hydrochloride

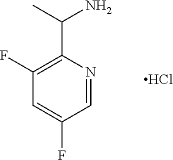

1-(3,5-Difluoropyridin-2-yl)-N-hydroxyethanimine (Intermediate 21, 9.73 g, 56.53 mmol) was added to water (113 ml) to form a suspension. Ammonium hydroxide (22.01 ml, 565.26 mmol) was added to the above solution, followed by ammonium acetate (5.23 g, 67.83 mmol). The mixture was heated at 50° C. and subsequently zinc (14.79 g, 226.11 mmol) was added portion wise, while maintaining the internal temperature below 65° C. After the addition was complete, the reaction mixture was stirred at 50° C. for 3 hours. Solid NaCl and EtOAc were added to quench the reaction. The reaction mixture was stirred for 1 hour at room temperature, was then filtered through diatomaceous earth (Celite®), and rinsed with EtOAc. The organic layer was washed with 5 ml 2.5% NaOH (aq.), followed by 10 ml NH₄OH. The organic layer was then washed with brine and dried with Na₂SO₄. The organic layer was concentrated under reduced pressure to obtain the title product as light yellow oil.

¹H NMR (400 MHz, MeOD) δ ppm 1.62 (d, J=6.82 Hz, 3H), 4.86 (q, J=6.82 Hz, 1H), 7.75 (ddd, J=10.11, 8.34, 2.27 Hz, 1H), 8.49 (d, J=2.27 Hz, 1H).

The hydrochloride salt was prepared by dissolving the oil in anhydrous methanol, adding 4N HCl in dioxane, allowing the solution to stir for 1 hour and subsequent evaporation of the volatiles under reduced pressure. The hydrochloride salt can be used in subsequent step without any further purification.

### Intermediate 25

### 6-Chloro-N-[1-(3,5-difluoropyridin-2-yl)ethyl]-N′-(1-methyl-1H-imidazol-4-yl)-1,3,5-triazine-2,4-diamine

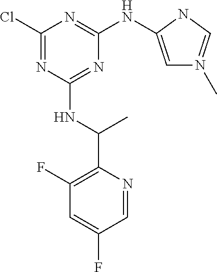

To a solution of 4,6-dichloro-N-(1-methyl-1H-imidazol-4-yl)-1,3,5-triazin-2-amine (Intermediate 2, 130 mg, 0.53 mmol) in ethanol (1490 μl) was added 1-(3,5-difluoropyridin-2-yl)ethanamine hydrochloride (Intermediate 24, 103 mg, 0.53 mmol) followed by DIPEA (278 μl, 1.59 mmol). The resulting mixture was stirred at 25° C. for 12 hours. The title product was obtained after filtration of the reaction mixture and drying under reduced pressure. The title product was used in the subsequent step without any further purification.

LCMS: 367 [M+H]⁺.

### Intermediate 26

### 1-(²H₃)Methyl-4-nitro-1H-imidazole

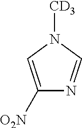

4-Nitro-1H-imidazole (500 mg) and CD₃I (0.3 ml) were reacted using a procedure similar to the one described for the synthesis of Intermediate 1, providing the title product (382 mg).

LCMS: 131 [M+H]⁺

### Intermediate 27

### 4,6-Dichloro-N-[1-(²H₃)methyl-1H-imidazol-4-yl]-1,3,5-triazin-2-amine

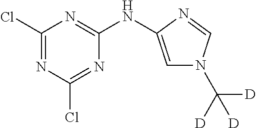

1-(²H₃)Methyl-4-nitro-1H-imidazole (Intermediate 26, 260 mg, 2.00 mmol) was dissolved in ethanol (3.439 mL) and Pd/C (10 wt %, Degussa®) (53.2 mg, 0.05 mmol) was added. The reaction was subjected to 1 atm of hydrogen. After 3 hours, TLC analysis confirmed the consumption of starting material, hence the reaction mixture was filtered through diatomaceous earth (Celite®), and the filtrate was cooled to 0° C. TEA (0.557 mL, 4.00 mmol) and 2,4,6-trichloro-1,3,5-triazine (368 mg, 2.00 mmol) were then added, and the reaction was allowed to slowly warm to room temperature overnight. The reaction mixture was filtered providing the title product as a tan solid (211 mg).

LCMS: 249 [M+H]⁺.

### Intermediate 28

### 6-Chloro-N-[1-(3,5-difluoropyridin-2-yl)ethyl]-N′-[1-(²H₃)methyl-1H-imidazol-4-yl]-1,3,5-triazine-2,4-diamine

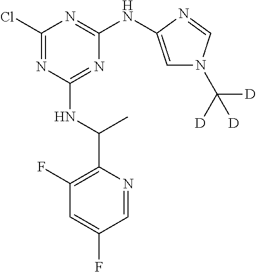

1-(3,5-Difluoropyridin-2-yl)ethanamine hydrochloride (Intermediate 24, 580 mg, 2.51 mmol) was suspended in acetonitrile (3.609 mL) and TEA (1.272 mL, 9.13 mmol) and 4,6-Dichloro-N-[1-(²H₃)methyl-1H-imidazol-4-yl]-1,3,5-triazin-2-amine (Intermediate 27, 566 mg, 2.28 mmol) were added. The reaction was stirred overnight at room temperature. The reaction mixture was filtered providing the title product as an off-white solid (1.320 g).

LCMS: 369 [M+H]⁺.

### Intermediate 29

### (4-Nitro-1H-imidazol-1-yl)acetonitrile

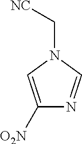

A mixture of 4-nitro-1H-imidazole (2.0 g, 17.69 mmol), 2-chloroacetonitrile (1.335 g, 17.69 mmol), and K₂CO₃ (3.67 g, 26.53 mmol) in acetonitrile (20 mL) were heated at 65° C. overnight. Evaporation of the volatiles under reduced pressure gave a residue that was partitioned between DCM and water. The organic phase was washed with water and dried (MgSO₄). After filtration, the volatiles were removed under reduced pressure to give the title product (1.89 g, 70%).

¹H NMR (400 MHz, DMSO-d₆) δ ppm 8.55 (d, 1H), 8.02 (d, 1H), 5.44 (s, 2H).

LCMS: 153 [M+H]⁺.

### Intermediate 30

### [4-[(4,6-Dichloro-1,3,5-triazin-2-yl)amino]-1H-imidazol-1-yl]acetonitrile

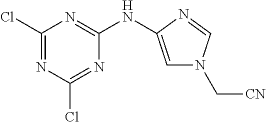

(4-Nitro-1H-imidazol-1-yl)acetonitrile (Intermediate 29, 304 mg, 2.00 mmol) was dissolved in ethanol (20 mL) and Pd/C (10 wt %, Degussa®, 53.2 mg, 0.05 mmol) was added. The reaction was subjected to 1 atm of hydrogen over night. The reaction mixture was filtered through diatomaceous earth (Celite®) and the filtrate was cooled to 0° C. 2,4,6-trichloro-1,3,5-triazine (369 mg, 2 mmol) and TEA (0.558 mL, 4.00 mmol) were then added and the reaction was allowed to warm to room temperature slowly overnight. The title product (443 mg, 82%) was obtained after filtration.

¹H NMR (400 MHz, DMSO-d₆) δ ppm 11.56 (s, 1H), 7.71 (s, 1H), 7.45 (s, 1H), 5.41 (s, 2H).

LCMS: 271 [M+H]⁺.

### Intermediate 31

### {4-[(4-Chloro-6-{[(1S)-1-(5-fluoropyrimidin-2-yl)ethyl]amino}-1,3,5-triazin-2-yl)amino]-1H-imidazol-1-yl}acetonitrile

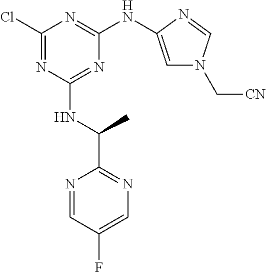

A mixture of {4-[(4,6-dichloro-1,3,5-triazin-2-yl)amino]-1H-imidazol-1-yl}acetonitrile (Intermediate 30, 0.423 g, 1.57 mmol), (1S)-1-(5-fluoropyrimidin-2-yl)ethanamine hydrochloride (Intermediate 11, 0.306 g, 1.72 mmol), and DIPEA (0.684 mL, 3.92 mmol) in ethanol (20 ml) was stirred at room temperature overnight. Evaporation of the volatiles under reduced pressure and subsequent purification by column chromatography (ISCO, 5% MeOH/0.5% NH₄OH in DCM) gave the title product (323 mg, 55%).

LCMS: 375 [M+H]⁺.

### Intermediate 32

### 1-(Methoxymethyl)-4-nitro-1H-imidazole

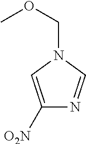

4-Nitro-1H-imidazole (2.0 g, 17.69 mmol) and 1-chloro-2-methoxymethane (2.85 g, 35.37 mmol) were reacted using a procedure similar to the one described for the synthesis of Intermediate 29, providing the title product as yellow solid (1.36 g, 48%).

¹H NMR (400 MHz, MeOD) δ ppm 8.28 (d, 1H), 7.92 (d, 1H), 5.43 (s, 2H), 3.36 (s, 3 H).

### Intermediate 33

### 4,6-Dichloro-N-[1-(methoxymethyl)-1H-imidazol-4-yl]-1,3,5-triazin-2-amine

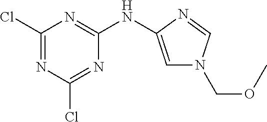

1-(Methoxymethyl)-4-nitro-1H-imidazole (Intermediate 32, 0.314 g, 2.00 mmol) was dissolved in ethanol (20 mL) and Pd/C (10 wt %, Degussa®, 0.053 g, 0.05 mmol) was added. The reaction was subjected to 1 atm of hydrogen for 3 hours. TLC indicated that the reaction went to completion, so the reaction mixture was filtered through diatomaceous earth (Celite®) and the filtrate was cooled to 0° C. 2,4,6-trichloro-1,3,5-triazine (0.369 g, 2 mmol) and TEA (0.558 mL, 4.00 mmol) were then added and the reaction was allowed to warm to room temperature slowly overnight. The reaction mixture was used directly to the next step.

LCMS: 276 [M+H]⁺.

### Intermediate 34

### 6-Chloro-N-[(1S)-1-(5-fluoropyrimidin-2-yl)ethyl]-N′-[1-(methoxymethyl)-1H-imidazol-4-yl]-1,3,5-triazine-2,4-diamine

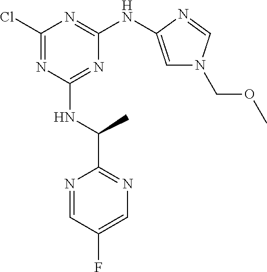

4,6-Dichloro-N-[1-(methoxymethyl)-1H-imidazol-4-yl]-1,3,5-triazin-2-amine (Intermediate 33, 0.550 g, 2 mmol), (1S)-1-(5-fluoropyrimidin-2-yl)ethanamine hydrochloride (Intermediate 11, 0.355 g, 2.00 mmol) were reacted using a procedure similar to the one described for the synthesis of Intermediate 31, providing the title product (525 mg, 61%).

LCMS: 380 [M+H]⁺.

### Intermediate 35

### 1-Isopropyl-4-nitro-1H-imidazole

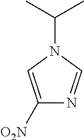

4-Nitro-1H-imidazole (2.0 g, 17.69 mmol) and 2-iodopropane (3.01 g, 17.69 mmol), were reacted using a procedure similar to the one described for the synthesis of Intermediate 29, providing the title product (2.12 g, 77%).

¹H NMR (400 MHz, CHLOROFORM-d) d ppm 7.82 (d, 1H), 7.51 (d, 1H), 4.38-4.51 (m, 1H), 1.58 (d, 6H).

LCMS: 156 [M+H]⁺.

### Intermediate 36

### 4,6-Dichloro-N-(1-isopropyl-1H-imidazol-4-yl)-1,3,5-triazin-2-amine

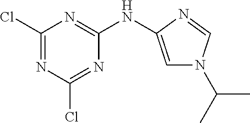

To a mixture of 1-isopropyl-4-nitro-1H-imidazole (Intermediate 35, 0.326 g, 2.10 mmol) in ethanol (20 mL), Pd/C (10 wt %, Degussa®, 0.053 g, 0.05 mmol) was added. The reaction was subjected to 1 atm of hydrogen for 3 hours. TLC indicated that the reaction went to completion, so the reaction mixture was filtered through diatomaceous earth (Celite®) and the filtrate was cooled to 0° C. 2,4,6-trichloro-1,3,5-triazine (0.369 g, 2 mmol) and TEA (0.558 mL, 4.00 mmol) were then added and the reaction was allowed to warm to room temperature slowly overnight. The reaction mixture was used directly to the next step. LCMS: 274 [M+H]⁺.

### Intermediate 37

### 6-Chloro-N-[(1S)-1-(5-fluoropyrimidin-2-yl)ethyl]-N′-(1-isopropyl-1H-imidazol-4-yl)-1,3,5-triazine-2,4-diamine

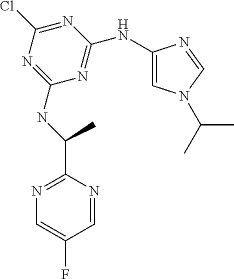

4,6-Dichloro-N-(1-isopropyl-1H-imidazol-4-yl)-1,3,5-triazin-2-amine (Intermediate 36, 0.546 g, 2 mmol) and (1S)-1-(5-fluoropyrimidin-2-yl)ethanamine hydrochloride (Intermediate 11, 0.355 g, 2.00 mmol) were reacted using a procedure similar to the one described for the synthesis of Intermediate 31, providing the title product.

LCMS: 378 [M+H]⁺.

### Intermediate 38

### 5-Nitro-1-{[2-(trimethylsilyl)ethoxy]methyl}-1H-imidazole and/or 4-Nitro-1-{[2-(trimethylsilyl)ethoxy]methyl}-1H-imidazole

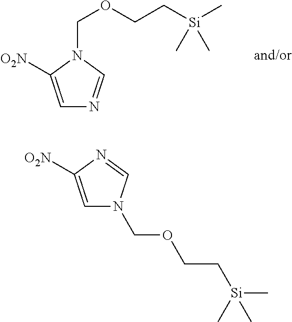

To a solution of 5-nitro-1H-imidazole (3 g, 26.53 mmol) in DMF (100 mL), at 0° C., was added sodium hydride (1.215 g, 27.86 mmol, 60% w/w in mineral oil). The resulting mixture was stirred for 30 mins at this temperature, whereupon (2-(chloromethoxy)ethyl)trimethylsilane (5.17 mL, 29.18 mmol) was added. The solution was allowed to warm to room temperature and stirred additional 1 hr. The mixture was partitioned water and EtOAc. The organic layer was dried (MgSO₄), filtered and evaporation under reduced pressure gave a residue. Purification by column chromatography (ISCO) gave the title product (2.75 g).

### Intermediate 39

### 1-{[2-(Trimethylsilyl)ethoxy]methyl}-1H-imidazol-5-amine and/or 1-{[2-(trimethylsilyl)ethoxy]methyl}-1H-imidazol-4-amine

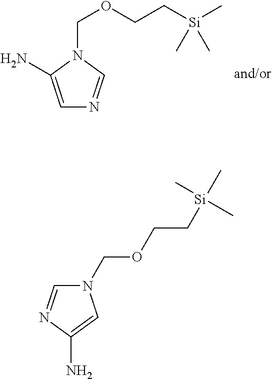

To a solution of 5-nitro-1-{[2-(trimethylsilyl)ethoxy]methyl}-1H-imidazole and/or 4-nitro-1-{[2-(trimethylsilyl)ethoxy]methyl}-1H-imidazole (Intermediate 38, 2.75 g, 11.30 mmol) in ethanol (50 mL) was added palladium on carbon (0.55 g, 0.52 mmol). The mixture was stirred overnight under a hydrogen atmosphere. The mixture was filtered and evaporation of the filtrate under reduced pressure gave the title product that was used in the next step without any further purification.

### Intermediate 40

### 4,6-Dichloro-N-(1-{[2-(trimethylsilyl)ethoxy]methyl}-1H-imidazol-5-yl)-1,3,5-triazin-2-amine and/or 4,6-Dichloro-N-(1-{[2-(trimethylsilyl)ethoxy]methyl}-1H-imidazol-4-yl)-1,3,5-triazin-2-amine

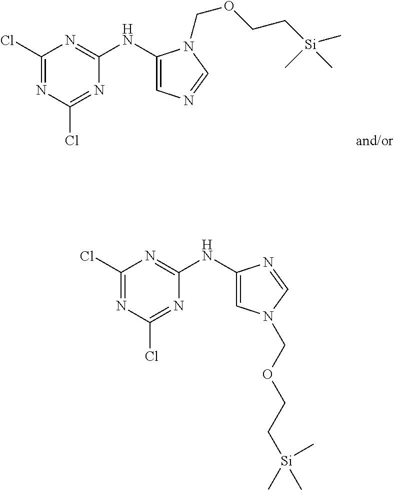

1-{[2-(Trimethylsilyl)ethoxy]methyl}-1H-imidazol-5-amine and/or 1-{[2-(Trimethylsilyl)ethoxy]methyl}-1H-imidazol-4-amine (Intermediate 39, 694 mg, 3.25 mmol) and 2,4,6-trichloro-1,3,5-triazine (600 mg, 3.25 mmol) were reacted using a procedure similar to the one described for the synthesis of Intermediate 30, providing the title product (173 mg) after column chromatography purification (ISCO).

### Intermediate 41

### 6-Chloro-N-[(1S)-1-(5-fluoropyrimidin-2-yl)ethyl]-N′-(1-{[2-(trimethylsilyl)ethoxy]methyl}-1H-imidazol-5-yl)-1,3,5-triazine-2,4-diamine and/or

### 6-Chloro-N-[(1S)-1-(5-fluoropyrimidin-2-yl)ethyl]-N′-(1-{[2-(trimethylsilyl)ethoxy]methyl}-1H-imidazol-4-yl)-1,3,5-triazine-2,4-diamine

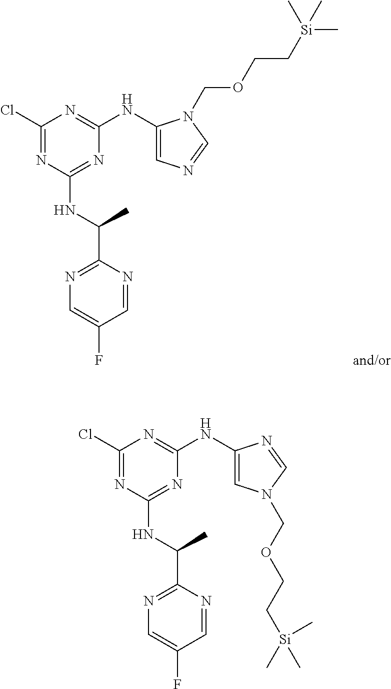

(1S)-1-(5-Fluoropyrimidin-2-yl)ethanamine hydrochloride (Intermediate 11, 85 mg, 0.48 mmol) and 4,6-dichloro-N-(1-{[2-(trimethylsilyl)ethoxy]methyl}-1H-imidazol-5-yl)-1,3,5-triazin-2-amine and/or 4,6-dichloro-N-(1-{[2-(trimethyl silyl)ethoxy]methyl}-1H-imidazol-4-yl)-1,3,5-triazin-2-amine (Intermediate 40, 173 mg, 0.48 mmol) were reacted using a procedure similar to the one described for the synthesis of Intermediate 31, providing the title product (224 mg) after purification by column chromatography (ISCO, 0→80% ethyl acetate in hexanes).

LCMS: 467 [M+H]⁺.

### Intermediate 42

### tert-Butyl [2-({4-chloro-6-[(1-methyl-1H-imidazol-4-yl)amino]-1,3,5-triazin-2-yl}amino)-2-(4-fluorophenyl)ethyl]carbamate

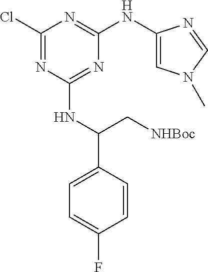

To a solution of 4,6-dichloro-N-(1-methyl-1H-imidazol-4-yl)-1,3,5-triazin-2-amine (Intermediate 2, 120 mg, 0.49 mmol) in acetonitrile (2277 μl) was added tert-butyl 2-amino-2-(4-fluorophenyl)ethylcarbamate (125 mg, 0.49 mmol)followed by DIPEA (171 μl, 0.98 mmol). The resulting colored solution was stirred overnight at room temperature. TLC analysis indicated complete consumption of the starting material. The reaction mixture was used in the subsequent step.

LCMS: 463 [M+H]⁺.

### Intermediate 43

### 6-Chloro-N-[(4-fluorophenyl)(1-methyl-1H-imidazol-2-yl)methyl]-N′-(1-methyl-1H-imidazol-4-yl)-1,3,5-triazine-2,4-diamine

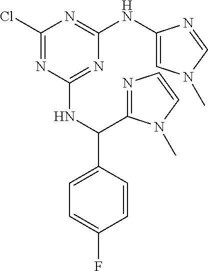

To a solution of 4,6-dichloro-N-(1-methyl-1H-imidazol-4-yl)-1,3,5-triazin-2-amine (Intermediate 2, 120 mg, 0.49 mmol) in acetonitrile (2277 μl) was added (4-fluorophenyl)(1-methyl-1H-imidazol-2-yl)methanamine (100 mg, 0.49 mmol)followed by DIPEA (171 μl, 0.98 mmol). The resulting colored solution was stirred overnight at room temperature. TLC analysis indicated complete consumption of the starting material. The reaction mixture was used in the subsequent step.

LCMS: 415 [M+H]⁺.

### Intermediate 44

### 6-Chloro-N-[cyclopentyl(4-fluorophenyl)methyl]-N′-(1-methyl-1H-imidazol-4-yl)-1,3,5-triazine-2,4-diamine

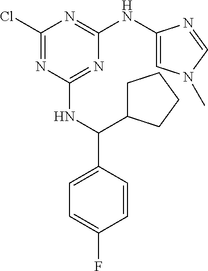

Cyclopentyl(4-fluorophenyl)methanamine (387 mg, 2.00 mmol) and 4,6-dichloro-N-(1-methyl-1H-imidazol-4-yl)-1,3,5-triazin-2-amine (Intermediate 2, 490 mg, 2 mmol) were reacted using a procedure similar to the one described for the synthesis of Intermediate 31, providing the title product (564 mg).

¹H NMR (300 MHz, MeOD) δ ppm 10.09 (s, 2H), 7.34-7.55 (m, 3H), 7.07-7.19 (m, 3 H), 4.71 (q., 1H), 3.65 (s, 3H), 3.12 (m, 1H), 1.40-2.38 (m, 8H).

LCMS: 402 [M+H]⁺.

### Intermediate 45

### 4-[(1S)-1-({4-Chloro-6-[(1-methyl-1H-imidazol-4-yl)amino]-1,3,5-triazin-2-yl}amino)ethyl]benzonitrile

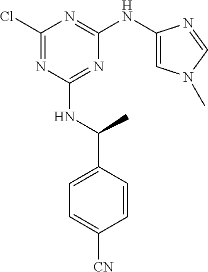

(S)-4-(1-Aminoethyl)benzonitrile hydrochloride (224 mg, 1.22 mmol) and 4,6-dichloro-N-(1-methyl-1H-imidazol-4-yl)-1,3,5-triazin-2-amine (Intermediate 2, 300 mg, 1.22 mmol) were reacted using a procedure similar to the one described for the synthesis of Intermediate 31, providing the title product (90 mg).

LCMS: 355 [M+H]⁺.

### Intermediate 46

### 6-Chloro-N-[(1S)-1-(4-chlorophenyl)ethyl]-N′-(1-methyl-1H-imidazol-4-yl)-1,3,5-triazine-2,4-diamine

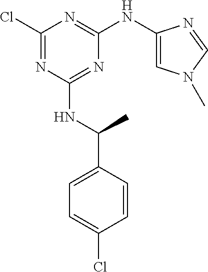

(S)-1-(4-Chlorophenyl)ethanamine (318 mg, 2.04 mmol) and 4,6-dichloro-N-(1-methyl-1H-imidazol-4-yl)-1,3,5-triazin-2-amine (Intermediate 2, 500 mg, 2.04 mmol) were reacted using a procedure similar to the one described for the synthesis of Intermediate 31, providing the title product (743 mg).

LCMS: 365 [M+H]⁺.

### Intermediate 47

### 6-Chloro-N-[(1S)-1-(4-fluorophenyl)ethyl]-N′-(1-methyl-1H-imidazol-4-yl)-1,3,5-triazine-2,4-diamine

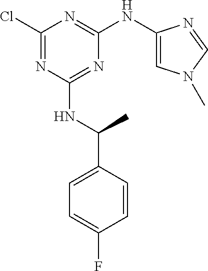

(S)-1-(4-Fluorophenyl)ethanamine (284 mg, 2.04 mmol) and 4,6-dichloro-N-(1-methyl-1H-imidazol-4-yl)-1,3,5-triazin-2-amine (Intermediate 2, 500 mg, 2.04 mmol) were reacted using a procedure similar to the one described for the synthesis of Intermediate 31, providing the title product (709 mg).

LCMS: 348 [M+H]⁺.

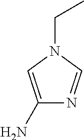

### Intermediate 48

### 1-Ethyl-1H-imidazol-5-amine

To a mixture of 4-nitro-1H-imidazole (2 g, 17.69 mmol) and potassium carbonate (3.67 g, 26.53 mmol) in acetonitrile (20 mL) was added iodoethane (1.713 mL, 21.22 mmol). The resulting reaction mixture was heated to 65° C. overnight, filtered and evaporation of the filtrate under reduced pressure gave a residue (1.2 g). Purification by column chromatography (ISCO) gave 1-ethyl-4-nitro-1H-imidazole (0.955 g, 6.77 mmol) that was re-dissolved in ethanol (35 mL). Palladium on carbon (0.191 g, 0.18 mmol) was added and the mixture was stirred at room temperature under hydrogen atmosphere for 3 hours. The mixture was filtered, the volatiles evaporated under reduced pressure (water bath <30° C.) and the title product was used I the next step without any further purification.

### Intermediate 49

### 4,6-Dichloro-N-(1-ethyl-1H-imidazol-4-yl)-1,3,5-triazin-2-amine

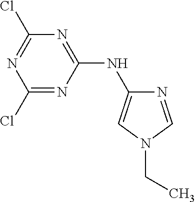

To a solution of 1-ethyl-1H-imidazol-5-amine (Intermediate 48, 362 mg, 3.25 mmol) in ethanol (14 mL), at 0° C., were added triethylamine (0.680 mL, 4.88 mmol) followed by 2,4,6-trichloro-1,3,5-triazine (600 mg, 3.25 mmol). The resulting reaction mixture was allowed to warm to room temperature overnight. The title product was obtained by filtration, washed with EtOH and dried overnight in a vacuum oven. The product (810 mg) was used in the subsequent step without any further purification.

LCMS: 260 [M+H]⁺.

### Intermediate 50

### 6-Chloro-N-[(1S)-1-(3,5-difluoropyridin-2-yl)ethyl]-N′-(1-ethyl-1H-imidazol-4-yl)-1,3,5-triazine-2,4-diamine

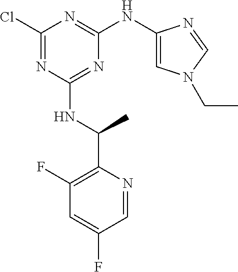

(1S)-1-(3,5-Difluoropyridin-2-yl)ethanamine, (R)-mandelic acid salt (Intermediate 22, 66 mg, 0.42 mmol) and 4,6-dichloro-N-(1-ethyl-1H-imidazol-4-yl)-1,3,5-triazin-2-amine (Intermediate 49, 109 mg, 0.42 mmol) were reacted using a procedure similar to the one described for the synthesis of Intermediate 31, providing the title product.

LCMS: 381 [M+H]⁺.

### Intermediate 51

### 1-Cyclopropyl-1H-imidazol-4-amine hydrochloride

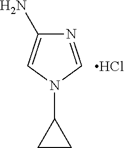

tert-Butyl 1-cyclopropyl-1H-imidazol-4-ylcarbamate (prepared with reference to PCT Pub. No. WO2008005956, 670 mg, 3.00 mmol) dissolved in methanol (15 mL) was treated with HCl (4N, 2.251 mL, 9.00 mmol) in dioxane. The solution was stirred at room temperature for 5 hours whereupon the volatiles were evaporated under reduced pressure to give the title product that was used in the next step without any further purification.

### Intermediate 52

### 4,6-Dichloro-N-(1-cyclopropyl-1H-imidazol-4-yl)-1,3,5-triazin-2-amine

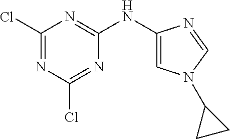

To a solution of 1-cyclopropyl-1H-imidazol-4-amine hydrochloride (Intermediate 51, 0.369 g, 3 mmol) in ethanol (15 mL), at 0° C., were added triethylamine (6.27 mL, 45.00 mmol) followed by 2,4,6-trichloro-1,3,5-triazine (0.553 g, 3.00 mmol). The resulting mixture was allowed to warm to room temperature overnight. The volatiles were evaporated under reduced pressure to give a residue, which was purified by column chromatography (ISCO, 0%→60% EtOAc in hexanes), furnishing the title product (579 mg).

LCMS: 271 [M+H]⁺.

### Intermediate 53

### 6-Chloro-N-(1-cyclopropyl-1H-imidazol-4-yl)-N′-[(1S)-1-(5-fluoropyrimidin-2-yl)ethyl]-1,3,5-triazine-2,4-diamine

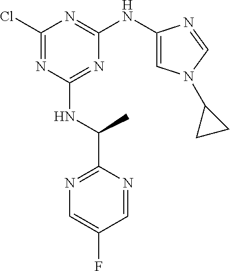

(1S)-1-(5-Fluoropyrimidin-2-yl)ethanamine hydrochloride (Intermediate 11, 379 mg, 2.14 mmol) and 4,6-dichloro-N-(1-cyclopropyl-1H-imidazol-4-yl)-1,3,5-triazin-2-amine (Intermediate 52, 579 mg, 2.14 mmol) were reacted using a procedure similar to the one described for the synthesis of Intermediate 31, providing the title product (396 mg) after column chromatography (ISCO, 0% 100% EtOAc in hexanes).

LCMS: 376 [M+H]⁺.

### Intermediate 54

### 3-(2-Bromoethyl)thiophene

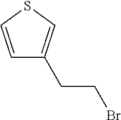

To a mixture of polymer supported triphenylphosphine (4.09 g, 15.60 mmol) in DCM (40 mL), at 0° C., was added bromine (0.804 mL, 15.60 mmol) and stirred for 15 minutes at this temperature. 2,6-Lutidine (2.181 mL, 18.72 mmol) was added and the reaction mixture was stirred at 0° C. for 0.5 hours. 3-(2-hydroxyethyl)thiophene was added and the mixture was stirred at 0° C. for 3 hours. The solids were filtered and the filtrate was evaporated under reduced pressure to give the title product (contaminated with small amount of 2,6-lutidine) that was used in the next step without any further purification.

### Intermediate 55

### 4-Nitro-1-[2-(3-thienyl)ethyl]-1H-imidazole

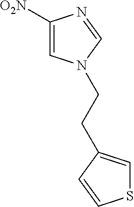

4-Nitro-1H-imidazole (1.313 g, 11.61 mmol) and 3-(2-bromoethyl)thiophene (Intermediate 54, 2.44 g, 12.77 mmol) were reacted using a procedure similar to the one described for the synthesis of Intermediate 1, providing the title product (2.21 g) after column chromatography (ISCO, 0%→50% EtOAc in hexanes).

LCMS: 224 [M+H]⁺.

### Intermediate 56

### 1-[2-(3-Thienyl)ethyl]-1H-imidazol-4-amine

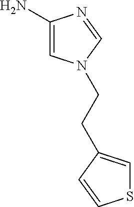

To a solution of 4-nitro-1-[2-(3-thienyl)ethyl]-1H-imidazole (Intermediate 55, 1.676 g, 7.51 mmol) in ethanol (37 mL) was added palladium on carbon (0.34 g, 0.32 mmol). The mixture was stirred overnight under a hydrogen atmosphere. The mixture was filtered and evaporation of the filtrate under reduced pressure gave the title product, which was used in the next step without further purification.

LCMS: 194 [M+H]⁺.

### Intermediate 57

### 4,6-Dichloro-N-{1-[2-(3-thienyl)ethyl]-1H-imidazol-4-yl}-1,3,5-triazin-2-amine

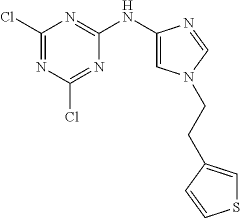

To a solution of 1-[2-(3-thienyl)ethyl]-1H-imidazol-4-amine (Intermediate 56, 739 mg, 3.82 mmol) and 2,4,6-trichloro-1,3,5-triazine (704 mg, 3.82 mmol) were reacted using a procedure similar to the one described for the synthesis of Intermediate 52, providing the product (1.077 g) after filtration of the reaction mixture.

LCMS: 342 [M+H]⁺.

### Intermediate 58

### 6-Chloro-N-[(1S)-1-(5-fluoropyrimidin-2-yl)ethyl]-N′-[1-(2-thien-3-ylethyl)-1H-imidazol-4-yl]-1,3,5-triazine-2,4-diamine

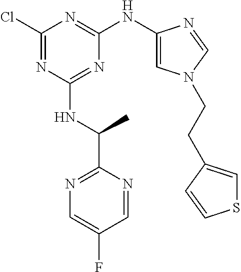

(1S)-1-(5-Fluoropyrimidin-2-yl)ethanamine hydrochloride (Intermediate 11, 260 mg, 1.47 mmol) and 4,6-Dichloro-N-{1-[2-(3-thienyl)ethyl]-1H-imidazol-4-yl}-1,3,5-triazin-2-amine (Intermediate 57, 500 mg, 1.47 mmol) were reacted using a procedure similar to the one described for the synthesis of Intermediate 31, providing the title product (130 mg) after column chromatography (ISCO, 0%→100% EtOAc in hexanes).

LCMS: 447 [M+H]⁺.

### Intermediate 59

### 4-Nitro-1-(2,2,2-trifluoroethyl)-1H-imidazole

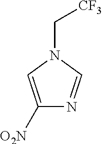

4-Nitro-1H-imidazole (2 g, 17.69 mmol) and 1,1,1-trifluoro-2-iodoethane (1.830 mL, 18.57 mmol) were reacted using a procedure similar to the one described for the synthesis of Intermediate 1, providing the title product (0.968 g) after column chromatography (ISCO).

### Intermediate 60

1-(2,2,2-Trifluoroethyl)-1H-imidazol-4-amine

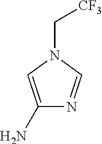

To a solution of 4-nitro-1-(2,2,2-trifluoroethyl)-1H-imidazole (Intermediate 59, 960 mg, 4.92 mmol) in ethanol (25 mL) was added palladium on carbon (192 mg, 0.18 mmol). The mixture was stirred overnight under a hydrogen atmosphere. The mixture was filtered and evaporation of the filtrate under reduced pressure gave the title product that was used in the next step without any further purification.

### Intermediate 61

### 4,6-Dichloro-N-[1-(2,2,2-trifluoroethyl)-1H-imidazol-4-yl]-1,3,5-triazin-2-amine

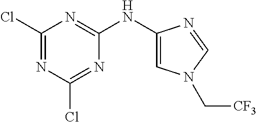

1-(2,2,2-Trifluoroethyl)-1H-imidazol-4-amine (Intermediate 60, 500 mg, 3.03 mmol) and 2,4,6-trichloro-1,3,5-triazine (0.558 g, 3.03 mmol) were reacted using a procedure similar to the one described for the synthesis of Intermediate 52, providing the product (840 mg) after filtration of the reaction mixture.

### Intermediate 62

### 6-Chloro-N-[(1S)-1-(5-fluoropyrimidin-2-yl)ethyl]-N′-[1-(2,2,2-trifluoro ethyl)-1H-imidazol-4-yl]-1,3,5-triazine-2,4-diamine

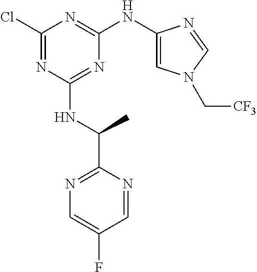

(1S)-1-(5-Fluoropyrimidin-2-yl)ethanamine hydrochloride (Intermediate 11, 284 mg, 1.6 mmol) and 4,6-Dichloro-N-[1-(2,2,2-trifluoroethyl)-1H-imidazol-4-yl]-1,3,5-triazin-2-amine (Intermediate 61, 500 mg, 1.60 mmol) were reacted using a procedure similar to the one described for the synthesis of Intermediate 31, providing the title product.

LCMS: 419 [M+H]⁺.

### Intermediate 63

### 6-Chloro-N-(1-ethyl-1H-imidazol-4-yl)-N′-[(1S)-1-(5-fluoropyrimidin-2-yl)ethyl]-1,3,5-triazine-2,4-diamine

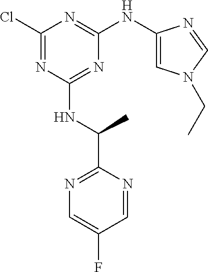

(1S)-1-(5-Fluoropyrimidin-2-yl)ethanamine hydrochloride (Intermediate 11, 343 mg, 1.93 mmol) and 4,6-dichloro-N-(1-ethyl-1H-imidazol-4-yl)-1,3,5-triazin-2-amine (Intermediate 49, 500 mg, 1.93 mmol) were reacted using a procedure similar to the one described for the synthesis of Intermediate 31, providing the title product.

LCMS: 365 [M+H]⁺.

### Example 2

### N-[(1R)-1-(3,5-Difluoropyridin-2-yl)-2-methoxyethyl]-6-[(2R,6S)-2,6-dimethylmorpholin-4-yl]-N′-(1-methyl-1H-imidazol-4-yl)-1,3,5-triazine-2,4-diamine

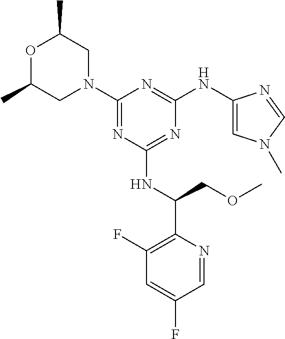

Cis-2,6-Dimethylmorpholine (0.034 mL, 0.28 mmol) was dissolved in ethanol (2.0 mL) and DIPEA (0.088 mL, 0.50 mmol) and 6-Chloro-N-[(1R)-1-(3,5-difluoropyridin-2-yl)-2-methoxyethyl]-N′-(1-methyl-1H-imidazol-4-yl)-1,3,5-triazine-2,4-diamine(Intermediate 6, 100 mg, 0.25 mmol) were added. The reaction mixture was then heated to 80° C. for 1 hour. The reaction mixture was concentrated in vacuo leaving a white solid (195 mg). This material was purified by ISCO (3-12% MeOH/DCM). Concentration of the fractions in vacuo provided the title product as a white solid (115.3 mg).

¹H NMR (300 MHz, MeOD) δ ppm 8.35 (br. s., 1H), 7.56 (t, 1H), 7.34 (s, 1H), 7.12 (br. s., 1H), 5.57-5.83 (m, 1H), 4.53 (d, 2H), 3.65-3.88 (m, 6H), 3.40-3.65 (m, 2H), 3.34 (s, 3H), 2.49 (t, 2H), 1.20 (d, 7H).

LCMS: 476 [M+H]⁺.

### Example 2

### N-[(1R)-1-(3,5-Difluoropyridin-2-yl)-2-methoxyethyl]-N′-(1-methyl-1H-imidazol-4-yl)-6-(2-methylmorpholin-4-yl)-1,3,5-triazine-2,4-diamine

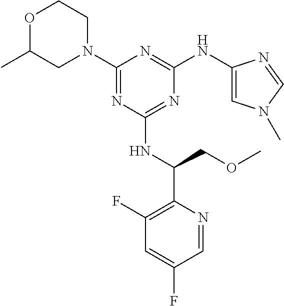

6-Chloro-N-[(1R)-1-(3,5-difluoropyridin-2-yl)-2-methoxyethyl]-N′-(1-methyl-1H-imidazol-4-yl)-1,3,5-triazine-2,4-diamine (Intermediate 6, 100 mg, 0.25 mmol) and 2-methylmorpholine (28.0 mg, 0.28 mmol) were reacted using a procedure similar to the one described for the synthesis of Example 1, providing the title product as a white solid (112.4 mg).

¹H NMR (300 MHz, MeOD) δ ppm 8.35 (br. s., 1H), 7.56 (t, 1H), 7.34 (d, 1H), 7.19 (br. s., 1H), 5.54-5.86 (m, 1H), 4.35-4.61 (m, 2H), 3.90 (d, 1H), 3.63-3.84 (m, 5H), 3.39-3.63 (m, 2H), 3.34 (s, 3H), 2.76-3.06 (m, 1H), 2.44-2.75 (m, 1H), 1.05-1.26 (m, 3H).

LCMS: 462 [M+H]⁺.

### Example 3

### N-[(1R)-1-(3,5-Difluoropyridin-2-yl)-2-methoxyethyl]-6-(2,2-dimethylmorpholin-4-yl)-N-(1-methyl-1H-imidazol-4-yl)-1,3,5-triazine-2,4-diamine

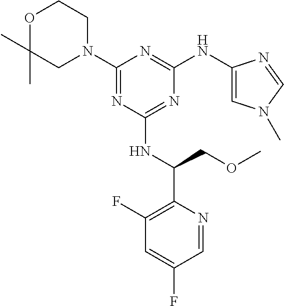

6-Chloro-N-[(1R)-1-(3,5-difluoropyridin-2-yl)-2-methoxyethyl]-N′-(1-methyl-1H-imidazol-4-yl)-1,3,5-triazine-2,4-diamine (Intermediate 6, 100 mg, 0.25 mmol) and 2,2-dimethylmorpholine, HCl (42.0 mg, 0.28 mmol) were reacted using a procedure similar to the one described for the synthesis of Example 1, providing the title product as a white solid (108.8 mg).

¹H NMR (300 MHz, MeOD) δ ppm 8.36 (br. s., 1H), 7.45-7.70 (m, 1H), 7.34 (d, 1H), 7.19 (br. s., 1H), 5.52-5.85 (m, 1H), 3.42-3.90 (m, 11H), 3.34 (s, 3H), 0.96-1.27 (m, 6H).

LCMS: 476 [M+H]⁺.

### Example 4

### N-[(1R)-1-(3,5-Difluoropyridin-2-yl)-2-methoxyethyl]-N′-(1-methyl-1H-imidazol-4-yl)-6-morpholin-4-yl-1,3,5-triazine-2,4-diamine

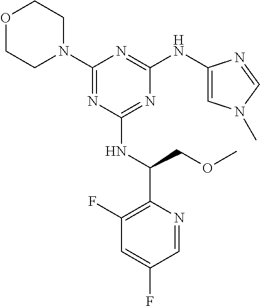

6-Chloro-N-[(1R)-1-(3,5-difluoropyridin-2-yl)-2-methoxyethyl]-N′-(1-methyl-1H-imidazol-4-yl)-1,3,5-triazine-2,4-diamine (Intermediate 6, 100 mg, 0.25 mmol) was dissolved in ethanol (2.0 mL) at 80° C., and morpholine (0.768 mL, 8.82 mmol) was added. The reaction mixture was then stirred at this temperature for 1 hour. The reaction mixture was then concentrated in vacuo leaving a white solid (333 mg). This material was purified by ISCO (3-12% MeOH/DCM). Concentration of the fractions in vacuo provided the title product as a pale yellow solid (112.5 mg).

¹H NMR (300 MHz, MeOD) δ ppm 8.36 (br. s., 1H), 7.46-7.67 (m, 1H), 7.34 (s, 1H), 7.19 (br. s., 1H), 5.58-5.83 (m, 1H), 3.51-3.89 (m, 15H), 3.34 (s, 3H).

LCMS: 448 [M+H]⁺.

### Example 5

### N-[(1S)-1-(5-Fluoropyrimidin-2-yl)ethyl]-N′-(1-methyl-1H-imidazol-4-yl)-6-morpholin-4-yl-1,3,5-triazine-2,4-diamine, Trifluoracetic acid salt

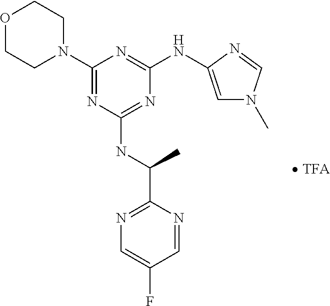

6-Chloro-N-[(1S)-1-(5-fluoropyrimidin-2-yl)ethyl]-N′-(1-methyl-1H-imidazol-4-yl)-1,3,5-triazine-2,4-diamine (Intermediate 12, 0.150 g, 0.43 mmol) in ethanol (2 mL) was treated with morpholine (2 ml, 22.96 mmol). The reaction mixture was stirred overnight at ambient temperature. Evaporation of the volatiles under reduced pressure gave an oil. Purification using a Gilson® column (5-95% MeCN/H₂O, 0.1% TFA), gave the title product (78.2 mg).

¹H NMR (300 MHz, MeOD) δ ppm 8.76 (s, 2H), 7.47 (s., 1H), 5.35 (q, 1H), 3.94 (s, 3 H), 3.61-3.84 (app. m, 8H), 1.65 (d, 3H).

LCMS: 401 [M+H]⁺.

### Example 6

### N-[(1S)-1-(5-Fluoropyrimidin-2-yl)ethyl]-6-morpholin-4-yl-N′-[1-(2-phenylethyl)-1H-imidazol-4-yl]-1,3,5-triazine-2,4-diamine, Trifluoroacetic Acid Salt

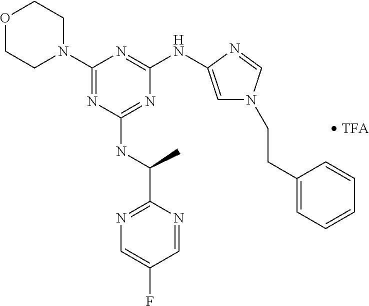

6-Chloro-N-[(1S)-1-(5-fluoropyrimidin-2-yl)ethyl]-N′-[1-(2-phenylethyl)-1H-imidazol-4-yl]-1,3,5-triazine-2,4-diamine (Intermediate 15, 310 mg, 0.70 mmol) and morpholine (4 mL, 45.91 mmol), were reacted using a procedure analogous to that described for the synthesis of Example 5, providing the title product (205.0 mg).

¹H NMR (300 MHz, MeOD) δ ppm 8.76 (s, 1H), 7.44 (br.s., 1H), 7.25-7.36 (m, 4H), 7.16-7.23 (m, 2H), 5.31 (q, 1H), 4.41-4.56 (m, 2H), 3.3.56-3.85 (m, 10H), 1.63 (d, 3H)

LCMS: 491 [M+H]⁺.

### Example 7

### 2-[(4-{[(1S)-1-(5-Fluoropyrimidin-2-yl)ethyl]amino}-6-morpholin-4-yl-1,3,5-triazin-2-yl)amino]-1,3-thiazole-5-carbonitrile

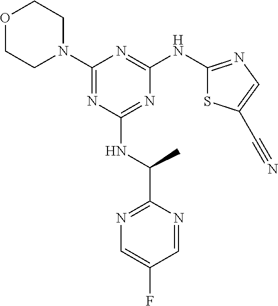

N-[(1S)-1-(5-Fluoropyrimidin-2-yl)ethyl]-6-morpholin-4-yl-1,3,5-triazine-2,4-diamine (Intermediate 18, 166 mg, 0.52 mmol), 2-chloro-1,3-thiazole-5-carbonitrile (Intermediate 16, 50 mg, 0.35 mmol), Xantphos® (20.01 mg, 0.03 mmol), Pd₂(dba)_{3 (}15.83 mg, 0.02 mmol) and Cs₂CO₃ (282 mg, 0.86 mmol) were combined in a microwave tube and vacuum purged. The tube was then charged with nitrogen and dioxane (1 mL) was added. The tube was evacuated again and placed under a nitrogen balloon and heated at 95° C. for 8 hours. The reaction mixture was concentrated in vacuo leaving a greenish-brown solid. This material was diluted with EtOAc and filtered through diatomaceous earth (Celite®). The organics were washed with water and brine and dried over Na₂SO₄. Concentration in vacuo gave an orange-brown solid. This material was purified by ISCO (0-10% MeOH/DCM). Concentration of the fractions in vacuo provided the title product as a yellow solid (127.9 mg).

¹H NMR (300 MHz, CHLOROFORM-d) δ ppm 12.58 (br. s., 1H), 9.30 (br. s., 1H), 8.43-8.75 (m, 2H), 7.98 (s, 1H), 5.34-5.59 (m, 1H), 3.49-4.10 (m, 8H), 1.66 (d, 3H).

LCMS: 429 [M+H]⁺.

### Example 8

### N-[(1S)-1-(5-Fluoropyrimidin-2-yl)ethyl]-N′-(5-methyl-1,3-thiazol-2-yl)-6-morpholin-4-yl-1,3,5-triazine-2,4-diamine

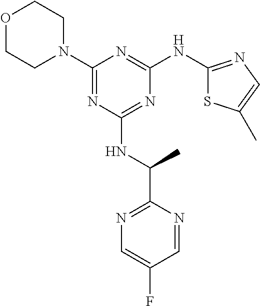

4-Chloro-N-[(1S)-1-(5-fluoropyrimidin-2-yl)ethyl]-6-morpholin-4-yl-1,3,5-triazin-2-amine (Intermediate 19, 100 mg, 0.29 mmol), 5-methylthiazol-2-amine (50.4 mg, 0.44 mmol), BINAP (18.33 mg, 0.03 mmol), Pd₂(dba)₃ (13.48 mg, 0.01 mmol) and Cs₂CO₃ (240 mg, 0.74 mmol) were combined in a microwave reaction tube and vacuum purged. The tube was then charged with nitrogen and dioxane (0.589 mL) was added. The tube was evacuated again and placed under a nitrogen balloon for 8 hours at 95° C. The reaction mixture was concentrated in vacuo leaving a brown solid (472 mg). This material was then re-dissolved in EtOAc, filtered through diatomaceous earth (Celite®), washed with water and brine and dried over Na₂SO₄. Concentration in vacuo gave a rust solid (272 mg). This material was purified by ISCO (55-95% EtOAc/Hex). Concentration of the fractions in vacuo provided the title product as a yellow solid (25.4 mg).

¹H NMR (300 MHz, CHLOROFORM-d) δ ppm 11.87 (br. s., 1H), 9.48 (br. s., 1H), 8.58 (s, 2H), 7.01 (s, 1H), 5.35 (app. q, 1H), 3.28-4.23 (m, 8H), 2.38 (s, 3H), 1.59 (d, 3H).

LCMS: 418 [M+H]⁺.

### Example 9

### 6-(4,4-Difluoropiperidin-1-yl)-N-[(1S)-1-(3,5-difluoropyridin-2-yl)ethyl]-N-(1-methyl-1H-imidazol-4-yl)-1,3,5-triazine-2,4-diamine

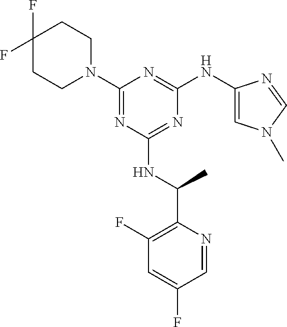

6-Chloro-N-[(1S)-1-(3,5-difluoropyridin-2-yl)ethyl]-N′-(1-methyl-1H-imidazol-4-yl)-1,3,5-triazine-2,4-diamine (Intermediate 23, 100 mg, 0.27 mmol) and 4,4-difluoropiperidine, HCl (47.3 mg, 0.30 mmol) were reacted using a procedure similar to the one described for the synthesis of Example 1, providing the title product as a white solid (101.6 mg).

¹H NMR (300 MHz, MeOD) δ ppm 8.32 (s, 1H), 7.47-7.67 (m, 1H), 7.34 (s, 1H), 7.06-7.30 (m, 1H), 5.37-5.69 (m, 1H), 4.62 (br. s., 1H), 3.87 (app. m., 4H), 3.71 (s, 3H), 1.89 (app m, 4H), 1.51 (d, 3H).

LCMS: 452 [M+H]⁺.

### Example 10

### N-[1-(3,5-Difluoropyridin-2-yl)ethyl]-N′-(1-methyl-1H-imidazol-4-yl)-6-morpholin-4-yl-1,3,5-triazine-2,4-diamine

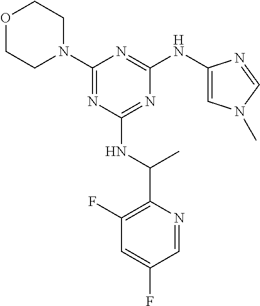

To a solution of 6-chloro-N-[1-(3,5-difluoropyridin-2-yl)ethyl]-N′-(1-methyl-1H-imidazol-4-yl)-1,3,5-triazine-2,4-diamine (Intermediate 25, 250 mg, 0.68 mmol) in ethanol (2153 μl) was heated to 70° C. and morpholine (119 μl, 1.36 mmol) was added. The initial cloudy solution became clear after 2 hours. The mixture was allowed to cool to room temperature. MeOH was added and the title product precipitated (75 mg, 26.4%) and was collected via filtration as a racemic mixture in the form of a white solid.

¹H NMR (300 MHz, MeOD) δ ppm 1.40 (d, 3H), 3.44-3.81 (m, 11H), 5.15-5.52 (m, 1 H), 7.05 (br. s., 1H), 7.24 (s, 1H), 7.45 (t, 1H), 8.22 (d, 1H).

LCMS: 367 [M+H]⁺.

### Column and Solvent Conditions

The R and S enantiomers of the title product were separated using a chiral HPLC column (Chiralpak® AD).

| | |
|---|---|
| Column dimensions: | 25 × 2 mm, 10μ |
| Mobile phase: | 100% 1:1 ethanol:methanol, |
| | 0.1% diethylamine (v/v/v) |
| Flow rate (ml/min): | 20 |
| Detection (nm): | 254 |
| Loading: | 40 mg/ml |

### Post Purification Purity Check

Sample purity was checked with a chiral column (Chiralpak® AD).

| Column dimensions: | 250 × 20 mm, 10μ |
|---|---|
| Mobile phase: | 100% 1:1 ethanol:methanol, |
| | 0.1% diethylamine (v/v/v) |
| Flow rate (ml/min): | 1 |
| Detection (nm): | 254 |

### Example 10(a)

### First Eluting Compound

### N-[(1R)-1-(3,5-Difluoropyridin-2-yl)ethyl]-N′-(1-methyl-1H-imidazol-4-yl)-6-morpholin-4-yl-1,3,5-triazine-2,4-diamine, Enantiomer (A)

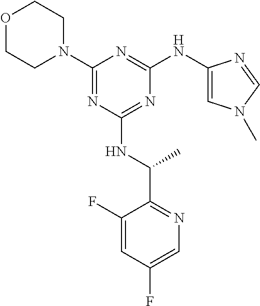

The first eluting compound had a retention time of ˜8 minutes, >98% ee.

¹H NMR (300 MHz, MeOD) δ ppm 1.40 (d, 3H) 3.47-3.75 (m, 11H) 5.21-5.62 (m, 1 H) 7.08 (br. s., 1H) 7.24 (s, 1H) 7.45 (t, 1H) 8.22 (d, 1H).

LCMS: 418 [M+H]⁺.

### Example 10(b)

### Second Eluting Compound

### N-[(1S)-1-(3,5-Difluoropyridin-2-yl)ethyl]-N′-(1-methyl-1H-imidazol-4-yl)-6-morpholin-4-yl-1,3,5-triazine-2,4-diamine, Enantiomer (B)

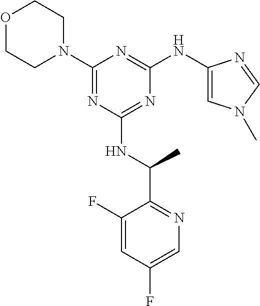

The second eluting compound had a retention time of ˜14 minutes, >98% ee.

¹H NMR (300 MHz, MeOD) δ ppm 1.40 (d, 3H) 3.41-3.73 (m, 11H) 5.27-5.59 (m, 1 H) 7.05 (br. s., 1H) 7.23 (s, 1H) 7.44 (t, 1H) 8.22 (d, 1H).

LCMS: 418 [M+H]⁺.

The compound of Example 10(b) may also be prepared via a chiral synthesis:

### Example 10(b)

### Via Chiral Synthesis

### N-[(1S)-1-(3,5-Difluoropyridin-2-yl)ethyl]-N-(1-methyl-1H-imidazol-4-yl)-6-morpholin-4-yl-1,3,5-triazine-2,4-diamine

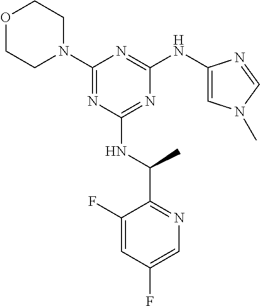

6-Chloro-N-[(1S)-1-(3,5-difluoropyridin-2-yl)ethyl]-N′-(1-methyl-1H-imidazol-4-yl)-1,3,5-triazine-2,4-diamine (Intermediate 23, 7.55 g, 20.59 mmol) and morpholine (17.93 ml, 205.86 mmol) were reacted using a procedure similar to the one described for the synthesis of Example 1, providing the title product as a white solid (6.235 g).

¹H NMR (300 MHz, MeOD) δ ppm 8.32 (s, 1H), 7.54 (t, 1H), 7.33 (s, 1H), 7.05-7.30 (m, 1H), 5.33-5.68 (m, 1H), 3.49-3.91 (m, 11H), 1.50 (d, 3H).

LCMS: 418 [M+H]⁺.

### Example 11

### N-[1-(3,5-Difluoropyridin-2-yl)ethyl]-N′-(1-methyl-1H-imidazol-4-yl)-6-(²H₈)morpholin-4-yl-1,3,5-triazine-2,4-diamine

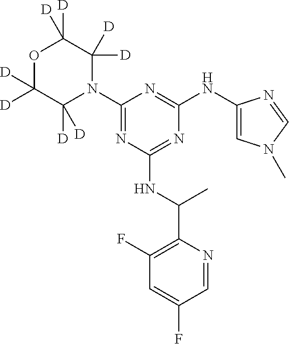

6-Chloro-N-[1-(3,5-difluoropyridin-2-yl)ethyl]-N′-(1-methyl-1H-imidazol-4-yl)-1,3,5-triazine-2,4-diamine (Intermediate 25, 400 mg, 1.09 mmol) was suspended in ethanol (4 mL) and TEA (0.608 mL, 4.36 mmol) was added. The reaction mixture was heated to 80° C. and morpholine-d8, HCl (287 mg, 2.18 mmol) was added. After 20 min, the reaction mixture was cooled to 0° C. and filtered leaving a white solid (198 mg). This material was separated between DCM and water and the organic layer was concentrated in vacuo providing the title product as a racemic mixture in the form of a white solid (110 mg).

¹H NMR (300 MHz, MeOD) δ ppm 8.32 (d, 1H) 7.54 (t, 1H) 7.32 (s, 1H) 7.03-7.28 (m, 1H) 5.30-5.67 (m, 1H) 3.70 (s, 3H) 1.50 (d, 3H).

LCMS: 426 [M+H]⁺.

### Column and Solvent Conditions

The R and S enantiomers of the title product were separated using a chiral HPLC column (Chiralpak® AD).

| Column dimensions: | 20 × 250 mm, 10μ | |
|---|---|---|
| Mobile phase: | 1:1 Methanol:Ethanol, 0.1% diethylamine | |
| Flow rate (ml/min): | 20 | mL/min |
| Detection (nm): | 220 | nm |

### Post Purification Purity Check:

Sample purity was checked with a chiral column (Chiralpak® AD).

| Column dimensions: | 4.6 × 250 mm, 10μ | |
|---|---|---|
| Mobile phase: | 1:1 Methanol:Ethanol, 0.1% diethylamine | |
| Flow: | 1.0 | mL/min |
| Detection: | 220 | nm |

### Example 11(a)

### First Eluting Compound

### N-[1-(3,5-Difluoropyridin-2-yl)ethyl]-N′-(1-methyl-1H-imidazol-4-yl)-6-(²H₈)morpholin-4-yl-1,3,5-triazine-2,4-diamine, Enantiomer (A)

The first eluting compound had a retention time of 8.255 minutes, >98% ee.

¹H NMR (300 MHz, MeOD) δ ppm 8.32 (d, 1H), 7.53 (t, 1H), 7.32 (s, 1H), 7.05-7.29 (m, 1H), 5.34-5.68 (m, 1H), 3.65 (s, 3H), 1.50 (d, 3H).

LCMS: 426 [M+H]⁺.

### Example 11(b)

### Second Eluting Compound

### N-[1-(3,5-Difluoropyridin-2-yl)ethyl]-N′-(1-methyl-1H-imidazol-4-yl)-6-(²H₈)morpholin-4-yl-1,3,5-triazine-2,4-diamine, Enantiomer (B)

The second eluting compound had a retention time of 14.875 minutes, >98% ee.

¹H NMR (300 MHz, MeOD) δ ppm 8.32 (d, 1H), 7.43-7.69 (m, 1H), 7.32 (s, 1H), 7.07-7.28 (m, 1H), 5.33-5.70 (m, 1H), 3.70 (s, 3H), 1.50 (d, 3H).

LCMS: 426 [M+H]⁺.

### Example 12

### N-[1-(3,5-Difluoropyridin-2-yl)ethyl]-N′-[1-(²H₃)methyl-1H-imidazol-4-yl]-6-morpholin-4-yl-1,3,5-triazine-2,4-diamine

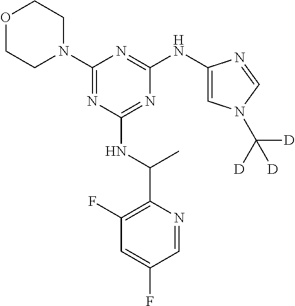

6-Chloro-N-[1-(3,5-difluoropyridin-2-yl)ethyl]-N′-[1-(²H₃)methyl-1H-imidazol-4-yl]-1,3,5-triazine-2,4-diamine (Intermediate 28, 500 mg, 1.35 mmol) was suspended in ethanol (5 mL) at 80° C. and morpholine (0.471 mL, 5.41 mmol) was added. After 2 hr, the reaction mixture was cooled to 0° C. and filtered leaving a white solid. This material was separated between DCM and water and the organic layer was concentrated in vacuo providing the title product as a racemic mixture in the form of a white solid (273 mg).

¹H NMR (300 MHz, MeOD) δ ppm 8.32 (d, 1H) 7.44-7.69 (m, 1H) 7.32 (d, 1H) 7.05-7.28 (m, 1H) 5.32-5.70 (m, 1H) 3.56-3.89 (m, 8H) 1.50 (d, 3H)

LCMS: 421 [M+H]⁺

### Column and Solvent Conditions

The R and S enantiomers of the title product were separated using a chiral HPLC column (Chiralpak® AD).

| Column dimensions: | 20 × 250 mm, 10μ | |
|---|---|---|
| Mobile phase: | 1:1 Methanol:Ethanol, 0.1% diethylamine | |
| Flow rate (ml/min): | 20 | mL/min |
| Detection (nm): | 220 | nm |

### Post Purification Purity Check

Sample purity was checked with a chiral column (Chiralpak® AD).

| Column dimensions: | 4.6 × 250 mm, 10μ | |
|---|---|---|
| Mobile phase: | 1:1 Methanol:Ethanol, 0.1% diethylamine | |
| Flow: | 1.0 | mL/min |
| Detection: | 220 | nm |

### Example 12(a)

### First Eluting Compound

### N-[1-(3,5-Difluoropyridin-2-yl)ethyl]-N′-[1-(²H₃)methyl-1H-imidazol-4-yl]-6-morpholin-4-yl-1,3,5-triazine-2,4-diamine, Enantiomer (A)

The first eluting compound had a retention time of 8.202 minutes, >98% ee.

¹H NMR (300 MHz, MeOD) δ ppm 8.32 (d, 1H), 7.54 (t, 1H), 7.32 (d, 1H), 7.04-7.28 (m, 1H), 5.30-5.71 (m, 1H), 3.53-3.87 (m, 8H), 1.50 (d, 3H)

LCMS: 421 [M+H]⁺

### Example 12(b)

### Second Eluting Compound

### N-[1-(3,5-Difluoropyridin-2-yl)ethyl]-N′-[1-(²H₃)methyl-1H-imidazol-4-yl]-6-morpholin-4-yl-1,3,5-triazine-2,4-diamine, Enantiomer (B)

The second eluting compound had a retention time of 14.630 minutes, >98% ee.

¹H NMR (300 MHz, MeOD) δ ppm 8.32 (d, 1H), 7.44-7.66 (m, 1H), 7.32 (d, 1H), 7.05-7.29 (m, 1H), 5.30-5.71 (m, 1H), 3.51-3.89 (m, 8H), 1.50 (d, 3H)

LCMS: 421 [M+H]⁺.

### Example 13

### N-[1-(3,5-Difluoropyridin-2-yl)ethyl]-N′-[1-(²H₃)methyl-1H-imidazol-4-yl]-6-(²H₈)morpholin-4-yl-1,3,5-triazine-2,4-diamine

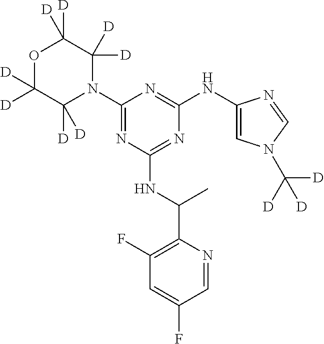

6-Chloro-N-[1-(3,5-difluoropyridin-2-yl)ethyl]-N′-[1-(²H₃)methyl-1H-imidazol-4-yl]-1,3,5-triazine-2,4-diamine (Intermediate 28, 500 mg, 1.35 mmol) was suspended in ethanol (5 mL) and TEA (0.754 mL, 5.41 mmol) was added. The reaction mixture was heated to 80° C. and morpholine-d8, HCl (356 mg, 2.70 mmol) was added. After 20 min, the reaction mixture was cooled to 0° C. and filtered leaving a white solid. This material was separated between DCM and water and the organic layer was concentrated in vacuo providing the title product as a racemic mixture in the form of a white solid (268 mg).

¹H NMR (300 MHz, MeOD) δ ppm 8.32 (d, 1H), 7.54 (t, 1H), 7.32 (d, 1H), 7.07-7.28 (m, 1H), 5.31-5.69 (m, 1H), 1.50 (d, 3H).

LCMS: 429 [M+H]⁺.

### Column and Solvent Conditions

The R and S enantiomers of the title product were separated using a chiral HPLC column (Chiralpak® AD).

| Column dimensions: | 20 × 250 mm, 10μ | |
|---|---|---|
| Mobile phase: | 1:1 Methanol:Ethanol, 0.1% diethylamine | |
| Flow rate (ml/min): | 20 | mL/min |
| Detection (nm): | 220 | nm |

### Post Purification Purity Check

Sample purity was checked with a chiral column (Chiralpak® AD).

| Column dimensions: | 4.6 × 250 mm, 10μ | |
|---|---|---|
| Mobile phase: | 1:1 Methanol:Ethanol, 0.1% diethylamine | |
| Flow: | 1.0 | mL/min |
| Detection: | 220 | nm |

### Example 13(a)

### First Eluting Compound

### N-[1-(3,5-Difluoropyridin-2-yl)ethyl]-N′-[1-(²H₃)methyl-1H-imidazol-4-yl]-6-(²H₈)morpholin-4-yl-1,3,5-triazine-2,4-diamine, Enantiomer (A)

The first eluting compound had a retention time of 8.181 minutes, >98% ee.

¹H NMR (300 MHz, MeOD) δ ppm 8.32 (d, 1H), 7.53 (t, 1H), 7.32 (d, 1H), 7.05-7.28 (m, 1H), 5.31-5.68 (m, 1H), 1.50 (d, 3H)

LCMS: 429 [M+H]⁺.

### Example 13(b)

### Second Eluting Compound

### N-[1-(3,5-Difluoropyridin-2-yl)ethyl]-N′-[1-(²H₃)methyl-1H-imidazol-4-yl]-6-(²H₈)morpholin-4-yl-1,3,5-triazine-2,4-diamine, Enantiomer (B)

The second eluting compound had a retention time of 14.467 minutes, >98% ee.

¹H NMR (300 MHz, MeOD) δ ppm 8.32 (d, 1H), 7.54 (t, 1H), 7.32 (d, 1H), 7.05-7.28 (m, 1H), 5.26-5.68 (m, 1H), 1.50 (d, 3H)

LCMS: 429 [M+H]⁺

### Example 14

### 6-(4,4-Difluoropiperidin-1-yl)-N-[(1S)-1-(5-fluoropyrimidin-2-yl)ethyl]-N′-(1-methyl-1H-imidazol-4-yl)-1,3,5-triazine-2,4-diamine

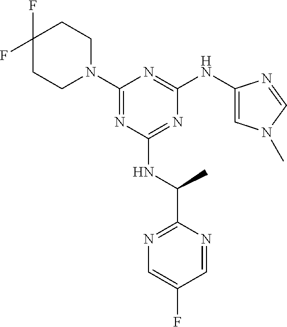

6-Chloro-N-[(1S)-1-(5-fluoropyrimidin-2-yl)ethyl]-N′-(1-methyl-1H-imidazol-4-yl)-1,3,5-triazine-2,4-diamine (Intermediate 12, 75 mg, 0.21 mmol) and 4,4-difluoropiperidine, HCl (37.2 mg, 0.24 mmol) were suspended in ethanol (1 mL) and DIPEA (0.075 mL, 0.43 mmol) was added. The reaction was then heated at 80° C. for 1 hour. The reaction mixture was concentrated in vacuo leaving a white semi-solid (182 mg). This material was purified by ISCO (0-10% MeOH/DCM). Concentration of the fractions in vacuo provided the title product as a white solid (71.1 mg).

¹H NMR (300 MHz, MeOD) δ ppm 8.70 (s, 2H), 6.97-7.51 (m, 2H), 5.11-5.45 (m, 1 H), 3.61-4.05 (m, 7H), 1.90 (br. s., 4H), 1.55 (d, 3H).

LCMS: 435 [M+H]⁺.

### Example 15

### {4-[(4-{[(1S)-1-(5-Fluoropyrimidin-2-yl)ethyl]amino}-6-morpholin-4-yl-1,3,5-triazin-2-yl)amino]-1H-imidazol-1-yl}acetonitrile

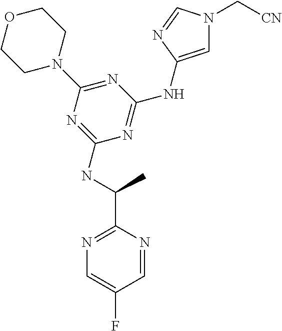

To a solution of {4-[(4-chloro-6-{[(1S)-1-(5-fluoropyrimidin-2-yl)ethyl]amino}-1,3,5-triazin-2-yl)amino]-1H-imidazol-1-yl}acetonitrile (Intermediate 31, 323 mg, 0.86 mmol) in ethanol (2.5 ml) was added morpholine (1742 mg, 20 mmol). The resulting reaction mixture was stirred at room temperature for 48 hours. The volatiles were removed under reduced pressure and the residue was purified by column chromatography (ISCO, 5% MeOH/0.5% NH₄OH in CH₂Cl₂) to yield the title product (302 mg, 82%). ¹H NMR (400 MHz, DMSO-d₆) δ ppm 8.80 (s, 2H), 8.47 (s, 1H), 7.42-7.58 (m, 1H), 7.31 (br. s., 1H), 6.93 (br. s., 1H), 5.20-5.35 (m, 1H), 3.64 (br. s., 4H), 3.59 (br. s., 4 H), 1.53 (d, 3H).

LCMS: 426 [M+H]⁺.

### Example 16

### N-[(1S)-1-(5-Fluoropyrimidin-2-yl)ethyl]-N′-[1-(methoxymethyl)-1H-imidazol-4-yl]-6-morpholin-4-yl-1,3,5-triazine-2,4-diamine

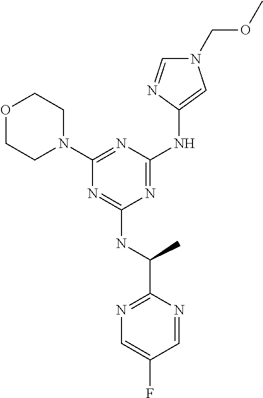

6-Chloro-N-[(1S)-1-(5-fluoropyrimidin-2-yl)ethyl]-N′-[1-(methoxymethyl)-1H-imidazol-4-yl]-1,3,5-triazine-2,4-diamine (Intermediate 34, 760 mg, 2.00 mmol) and morpholine (1742 mg, 20 mmol) were reacted using a procedure similar to the one described for the synthesis of Example 1, providing the title product (525 mg, 61%).

¹H NMR (400 MHz, DMSO-d₆) δ ppm 8.77 (s, 2H), 8.53 (br, 1H), 7.51 (d, 1H), 7.26 (br, 2H), 5.12-5.34 (m, 3H), 3.59 (app.m, 8H), 3.04 (s, 3H), 1.53 (d, 3H). LCMS: 431 [M+H]⁺.

### Example 17

### N-[(1S)-1-(5-Fluoropyrimidin-2-yl)ethyl]-N′-(1-isopropyl-1H-imidazol-4-yl)-6-morpholin-4-yl-1,3,5-triazine-2,4-diamine

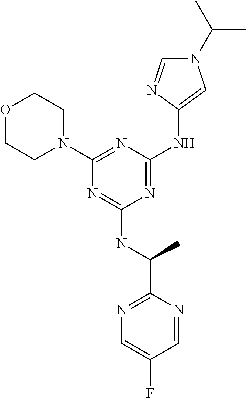

6-Chloro-N-[(1S)-1-(5-fluoropyrimidin-2-yl)ethyl]-N′-(1-isopropyl-1H-imidazol-4-yl)-1,3,5-triazine-2,4-diamine (Intermediate 37, 756 mg, 2 mmol) and morpholine (1742 mg, mmol) were reacted using a procedure similar to the one described for the synthesis of Example 1, providing the title product (476 mg, 56%).

¹H NMR (400 MHz, DMSO-d₆) δ ppm 8.76-8.81 (m, 2H), 8.20 (s, 1H), 7.37 (s, 1H), 7.20 (br. s., 1H), 6.92 (br, 1H), 5.26 (br m, 1H), 4.29-4.40 (m, 1H), 3.59 (app m, 8H), 1.53 (d, 3H), 1.44 (dd, 6H).

LCMS: 429 [M+H]⁺.

### Example 18

### N-[(1S)-1-(3,5-Difluoropyridin-2-yl)ethyl]-6-(3-fluoroazetidin-1-yl)-N′-(1-methyl-1H-imidazol-4-yl)-1,3,5-triazine-2,4-diamine

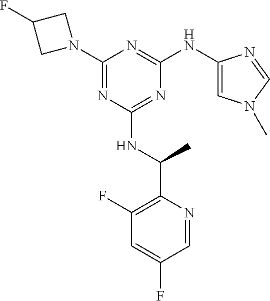

A solution of 6-chloro-N-[(1S)-1-(3,5-difluoropyridin-2-yl)ethyl]-N′-(1-methyl-1H-imidazol-4-yl)-1,3,5-triazine-2,4-diamine (Intermediate 23, 76 mg, 0.21 mmol) in ethanol (928 μl) was heated to 70° C. and DIPEA (109 μl, 0.62 mmol) followed by 3-fluoroazetidine (23.11 mg, 0.21 mmol) were added. The initial cloudy solution became clear after 1 hour. The mixture was allowed to cool to room temperature. The title product was isolated by filtration as a white solid (42.0 mg, 50.0%).

¹H NMR (300 MHz, DMSO-d₆) δ ppm 1.45 (d, 3H), 3.62 (s, 3H), 4.04 (m, 2H), 4.18-4.51 (m, 2H), 5.34 (m, 1.5H), 5.47-5.64 (m, 0.5H), 6.94 (br. s., 0.5H), 7.21-7.44 (m, 1.5H), 7.56 (br. s., 0.5H), 7.71-8.03 (m, 1H), 8.44 (d, 1H), 9.04 (br. s., 0.5H). LCMS: 406 [M+H]⁺.

### Example 19

### N-[(1S)-1-(3,5-Difluoropyridin-2-yl)ethyl]-6-(3-methoxyazetidin-1-yl)-N′-(1-methyl-1H-imidazol-4-yl)-1,3,5-triazine-2,4-diamine

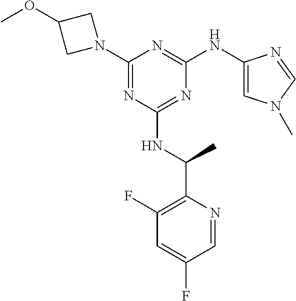

A solution of 6-chloro-N-[(1S)-1-(3,5-difluoropyridin-2-yl)ethyl]-N′-(1-methyl-1H-imidazol-4-yl)-1,3,5-triazine-2,4-diamine (Intermediate 23, 76 mg, 0.21 mmol) in ethanol (928 μl) was heated to 70° C. and DIPEA (109 μl, 0.62 mmol) followed by 3-methoxyazetidine (25.6 mg, 0.21 mmol) HCl were added. The initial cloudy solution became clear after 1 hour. The mixture was allowed to cool to room temperature. The title product was isolated by filtration as a white solid (45.0 mg, 52.0%).

¹H NMR (300 MHz, MeOD) δ ppm 1.53 (d, 3H), 3.32 (s, 3H), 3.73 (br. s., 3H), 3.82-3.98 (m, 2H), 4.13-4.48 (m, 3H), 5.37-5.68 (m, 1H), 7.21 (br. s., 0.5H), 7.35 (br. s, 1.5H), 7.48-7.71 (m, 1H), 8.35 (br. s., 1H).

LCMS: 418 [M+H]⁺.

### Example 20

### N-[(1S)-1-(5-Fluoropyrimidin-2-yl)ethyl]-6-(3-methoxyazetidin-1-yl)-N′-(1-methyl-1H-imidazol-4-yl)-1,3,5-triazine-2,4-diamine

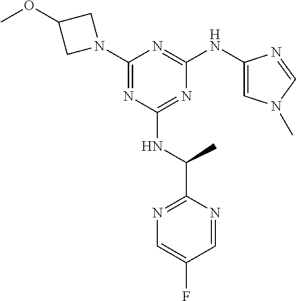

A solution of 6-chloro-N-[(1S)-1-(5-fluoropyrimidin-2-yl)ethyl]-N′-(1-methyl-1H-imidazol-4-yl)-1,3,5-triazine-2,4-diamine (Intermediate 12, 35 mg, 0.10 mmol) in ethanol (448 μl) was heated to 70° C. and DIPEA (52.4 μl, 0.30 mmol) followed by 3-methoxyazetidine, HCl (12.37 mg, 0.10 mmol) were added. The initial cloudy solution became clear after 1 hour. The mixture was allowed to cool to room temperature. Evaporation of the volatiles under reduced pressure gave a residue that was purified using a Gilson® column (5%-95% MeCN/H₂O, 15 min elution, 300 μL injections) afforded the title product (15.00 mg, 29.1%) as a trifluoroacetic acid salt.

¹H NMR (400 MHz, MeOD) δ ppm 1.61 (d, 3H), 3.35-3.40 (m, 2H), 3.89 (s, 1.5H), 3.97 (s, 1.5H), 3.99-4.10 (m, 1H), 4.19-4.52 (m, 2H), 5.35 (q, 1H), 7.12 (s, 0.5H), 7.30 (s, 0.5H), 8.14 (br. s., 0.5H), 8.48 (br. s., 0.5H), 8.75 (d, 2H).

LCMS: 401 [M+H]⁺.

### Example 21

### N-[(1S)-1-(3,5-Difluoropyridin-2-yl)ethyl]-6-(4-fluoropiperidin-1-yl)-N′-(1-methyl-1H-imidazol-4-yl)-1,3,5-triazine-2,4-diamine

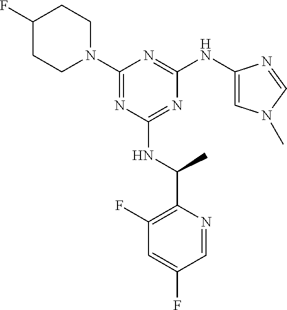

A solution of 6-chloro-N-[(1S)-1-(3,5-difluoropyridin-2-yl)ethyl]-N′-(1-methyl-1H-imidazol-4-yl)-1,3,5-triazine-2,4-diamine (Intermediate 23, 95 mg, 0.26 mmol) in ethanol (1159 μl)was heated to 70° C. and DIPEA (136 μl, 0.78 mmol) followed by 4-fluoropiperidine (36.2 mg, 0.26 mmol) were added. The initial cloudy solution became clear after 1 hour. The mixture was allowed to cool to room temperature. The title product was isolated by filtration as a white solid (55.0 mg, 49.0%).

¹H NMR (300 MHz, MeOD) δ ppm 1.52 (d, 3H), 1.61-2.03 (m, 4H), 3.72 (s, 3H), 3.75-3.94 (m, 4H), 4.65-4.80 (m, 1H), 5.28-5.64 (m, 1H), 7.16 (br. s., 1H), 7.35 (s, 1H), 7.57 (t, 1H), 8.34 (d, 1H).

LCMS: 434 [M+H]⁺.

### Example 22

### [(3R)-4-(4-{[(1S)-1-(3,5-difluoropyridin-2-yl)ethyl]amino}-6-[(1-methyl-1H-imidazol-4-yl)amino]-1,3,5-triazin-2-yl)morpholin-3-yl]methanol

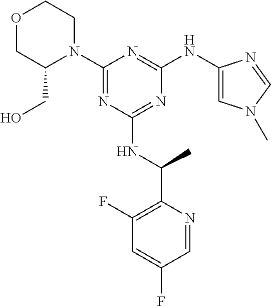

A solution of 6-chloro-N-[(1S)-1-(3,5-difluoropyridin-2-yl)ethyl]-N′-(1-methyl-1H-imidazol-4-yl)-1,3,5-triazine-2,4-diamine (Intermediate 23, 66 mg, 0.18 mmol) in BuOH (837 μl) was heated to 100° C. and DIPEA (62.9 μl, 0.36 mmol) followed by (R)-morpholin-3-ylmethanol (27.6 mg, 0.18 mmol) were added. The initial cloudy solution became clear after 1 hour. The mixture was allowed to heat o/n at 100° C. The volatiles were removed under reduced pressure and the residue was purified by column chromatography (ISCO, 0%/5%/10% MeOH-DCM) afforded the title product as a white solid (74.0 mg, 92%).

¹H NMR (400 MHz, MeOD) δ ppm 1.52 (d, 3H), 3.44-3.60 (m, 2H), 3.60-3.68 (m, 1H), 3.72 (br. s., 3H), 3.77-3.80 (m, 1H), 3.84-3.97 (m, 2H), 4.11 (d, 1H), 4.37 (d, 1 H), 4.49-4.61 (m, 1H), 5.36-5.79 (m, 1H), 7.19 (br. s., 1H), 7.40 (br. s., 1H), 7.56 (br. s., 1H), 8.35 (d, 1H).

LCMS: 448 [M+H]⁺.

### Example 23

### N-[(1S)-1-(5-Fluoropyrimidin-2-yl)ethyl]-N′-1H-imidazol-4-yl-6-morpholin-4-yl-1,3,5-triazine-2,4-diamine, Trifluoroacetic Acid Salt

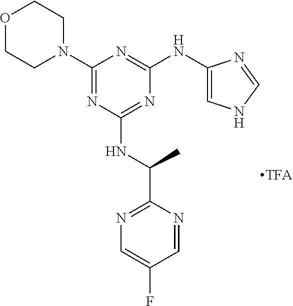

6-Chloro-N-[(1S)-1-(5-fluoropyrimidin-2-yl)ethyl]-N′-(1-{[2-(trimethylsilyl)ethoxy]methyl}-1H-imidazol-5-yl)-1,3,5-triazine-2,4-diamine and/or 6-Chloro-N-[(1S)-1-(5-fluoropyrimidin-2-yl)ethyl]-N′-(1-{[2-(trimethylsilyl)ethoxy]methyl}-1H-imidazol-4-yl)-1,3,5-triazine-2,4-diamine (Intermediate 41, 224 mg, 0.48 mmol) and morpholine (4 ml, 45.91 mmol) were reacted using a procedure similar to the one described for the synthesis of Example 1, the SEM protected product was dissolved in MeOH and HCl (4N in dioxane) was added. The resulting mixture was stirred at room temperature for 3 hours whereupon the volatiles were removed under reduced pressure. Purification using a Gilson® column (MeCN/0.1% TFA in water, 5%→70%) gave the title product (23.6 mg).

¹H NMR (300 MHz, MeOD) δ ppm 8.73 (s, 2H), 8.34 (s, 1H), 7.02 (s, 1H), 5.28 (m, 1H), 3.58-3.84 (m, 8H), 1.61 (d, 3H).

LCMS: 387 [M+H]⁺.

### Example 24

### tert-Butyl [2-(4-fluorophenyl)-2-({4-[(1-methyl-1H-imidazol-4-yl)amino]-6-morpholin-4-yl-1,3,5-triazin-2-yl}amino)ethyl]carbamate

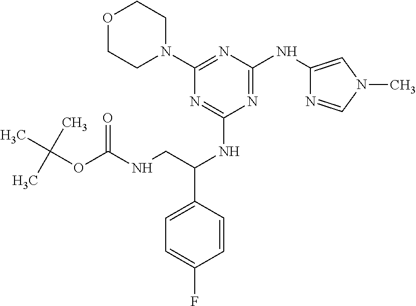

To a solution of tert-Butyl [2-({4-chloro-6-[(1-methyl-1H-imidazol-4-yl)amino]-1,3,5-triazin-2-yl}amino)-2-(4-fluorophenyl)ethyl]carbamate (Intermediate 42, 227 mg, 0.49 mmol) in MeCN was added morpholine (42.7 μl, 0.49 mmol) and the resulting cloudy solution was heated to 80° C. for 2 hours (the solids are dissolved when the external temperature reaches 70° C.). The mixture was allowed to cool to room temperature and the title product (16.90 mg, 6.72%) was collected by filtration under vacuum. The filtrate was evaporated under reduced pressure to give the title product as a racemic mixture in the form of a colored semi-solid. Purification by column chromatography (ISCO, 5%-10% MeOH/DCM) gave additional title product.

¹H NMR (300 MHz, MeOD) δ ppm 1.42 (s, 9H), 3.36 (s, 3H), 3.58-3.88 (m, 10H), 5.08-5.37 (m, 1H), 6.93-7.18 (m, 2H), 7.23-7.61 (m, 4H).

LCMS: 514 [M+H]⁺.

### Column and Solvent Conditions

The R and S enantiomers of the title product were chirally separated using a Chiralpak® AD column HPLC system.

| | |
|---|---|
| Column dimensions: | 20 × 250 mm, 10μ |
| Mobile phase: | 100% 1:1 ethanol:methanol, |
| | 0.1% diethylamine (v/v/v) |
| Flow rate (ml/min): | 20 |
| Detection (nm): | 220 |
| Loading: | 22 mg/inj |
| Concentration: | 11 mg/ml |

### Example 24(a)

### First Eluting Compound

### tert-Butyl [2-(4-fluorophenyl)-2-({4-[(1-methyl-1H-imidazol-4-yl)amino]-6-morpholin-4-yl-1,3,5-triazin-2-yl}amino)ethyl]carbamate, Enantiomer (A)

Yield: (16.90 mg, 6.72%)

The first eluting compound had a retention time of 7.05 minutes.

LCMS: 514 [M+H]⁺.

¹H NMR (300 MHz, MeOD) δ ppm 1.30 (s, 9H), 3.21 (s, 3H), 3.45-3.75 (m, 10H), 4.95-5.29 (m, 1H), 6.65-7.56 (m, 6H).

### Example 24(b)

### Second Eluting Compound

### tert-Butyl [2-(4-fluorophenyl)-2-({4-[(1-methyl-1H-imidazol-4-yl)amino]-6-morpholin-4-yl-1,3,5-triazin-2-yl}amino)ethyl]carbamate, Enantiomer (B)

Yield: (19.70 mg, 7.83%)

The second eluting compound had a retention time of 12.35 minutes.

¹H NMR (300 MHz, MeOD) δ ppm 1.30 (s, 9H), 3.21 (s, 3H), 3.44-3.71 (m, 10H), 4.95-5.24 (m, 1H), 6.85-7.03 (m, 2H), 7.09-7.42 (m, 4H).

LCMS: 514 [M+H]⁺.

The title product ee was determined using Chiral SFC:

| Column: | Chirapak AD-H | |
|---|---|---|
| Column dimensions: | 4.6 × 100 mm, 5μ | |
| Mobile phase: | 40% MeOH/DMEA | |
| Elution time: | 5 | ml/min |
| Flow rate (ml/min): | 5 | |
| Oven (° C.): | 35° | C. |
| Outlet Pressure (bar): | 120 | |
| Detection: | 254 | nm |

Enantiomeric excess (e.e.) for Example 24(b) was >98%, using area percent at 254 and 210 nm. The e.e. for Example 24 (a) was not determined.

### Example 25

### N-[(4-Fluorophenyl)(1-methyl-1H-imidazol-2-yl)methyl]-N′-(1-methyl-1H-imidazol-4-yl)-6-morpholin-4-yl-1,3,5-triazine-2,4-diamine

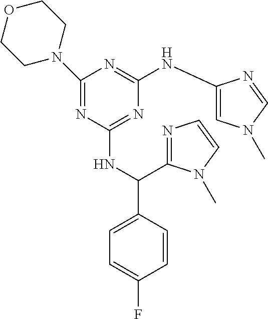

To a solution of 6-chloro-N-[(4-fluorophenyl)(1-methyl-1H-imidazol-2-yl)methyl]-N′-(1-methyl-1H-imidazol-4-yl)-1,3,5-triazine-2,4-diamine (Intermediate 43, 203 mg, 0.49 mmol) in acetonitrile (2 mL) was added morpholine (0.064 mL, 0.74 mmol). The resulting cloudy solution was heated to 80° C. for 2 hours whereupon became clear. The mixture was allowed to cool to room temperature whereupon a solid started precipitating. The mixture was filtered and the filtrate was dried under vacuum. The solid was identified as the title product as a racemic mixture (17.00 mg, 7.47%). Evaporation of the filtrate under reduced pressure provided a yellow semi-solid that was purified by ISCO (2%-10% MeOH/DCM) to afford additional title product (17.00 mg, 7.47%). ¹H NMR (300 MHz, MeOD) δ ppm 3.53 (app. s, 3H), 3.65 (s, 3H), 3.67-3.72 (m, 5H), 3.72-3.78 (m, 3H), 3.79-3.86 (m, 1H), 6.41-6.60 (m, 1H), 6.93 (d, 1H), 7.03-7.18 (m, 3H), 7.23-7.48 (m, 3H), 8.54 (s, 1H).

LCMS: 465 [M+H]⁺.

### Column and Solvent Conditions

The R and S enantiomers of the title product were chirally separated using a Chiralpak® AD column HPLC system.

| Column dimensions: | 20 × 250 mm, 10μ |
|---|---|
| Mobile phase: | 100% 1:1 ethanol:methanol, |
| | 0.1% diethylamine (v/v/v) |
| Flow rate (ml/min): | 20 |
| Detection (nm): | 220 |

### Example 25(a)

### First Eluting Compound

The first eluting compound was not isolated.

LCMS: 465 [M+H]⁺.

### Example 25(b)

### Second Eluting Compound

N-[(4-Fluorophenyl)(1-methyl-1H-imidazol-2-yl)methyl]-N′-(1-methyl-1H-imidazol-4-yl)-6-morpholin-4-yl-1,3,5-triazine-2,4-diamine, Enantiomer (B)

Yield: (17.00 mg, 7.47%).

¹H NMR (300 MHz, MeOD) δ ppm 3.53 (s, 3H), 3.56-3.61 (m, 7H), 3.60-3.72 (m, 4H), 6.27-6.53 (m, 1H), 6.81 (d, 1H), 6.89-7.05 (m, 3H), 7.15-7.38 (m, 3H).

LCMS: 465 [M+H]⁺.

The title product ee was not determined.

### Example 26

### N-[(1S)-1-(5-Fluoropyrimidin-2-yl)ethyl]-6-morpholin-4-yl-N′-1,3-thiazol-4-yl-1,3,5-triazine-2,4-diamine

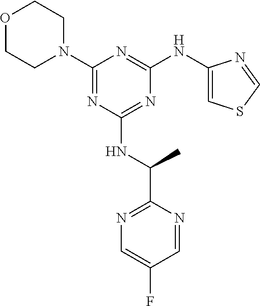

A screw-cap vial was charged with N-[(1S)-1-(5-Fluoropyrimidin-2-yl)ethyl]-6-morpholin-4-yl-1,3,5-triazine-2,4-diamine (Intermediate 18, 234 mg, 0.73 mmol), 4-bromothiazole (100 mg, 0.61 mmol), CS₂CO₃ (497 mg, 1.52 mmol), Xantphos® (35.3 mg, 0.06 mmol) and Pd₂(dba)₃ (27.9 mg, 0.03 mmol). The vial was flushed with nitrogen and dioxane (3048 μl) was added. The resulting mixture was heated to 100° C. for 12 hours. Evaporation of the volatiles under reduced pressure gave a residue that was purified by column chromatography (10%-20%-50%-100% EtOAc/hexanes) to give the title product (20.00 mg, 8.13%).

¹H NMR (300 MHz, MeOD) δ ppm 1.46 (d, 3H), 3.38-3.73 (m, 8H), 5.04-5.36 (m, 1 H), 7.37 (br. s., 0.5H), 7.56 (br. s., 0.5H), 8.59 (s, 2H), 8.64 (br. s., 1H).

LCMS: 404 [M+H]⁺.

### Example 27

### N-[Cyclopentyl(4-fluorophenyl)methyl]-N′-(1-methyl-1H-imidazol-4-yl)-6-morpholin-4-yl-1,3,5-triazine-2,4-diamine, Trifluoroacetic Acid Salt

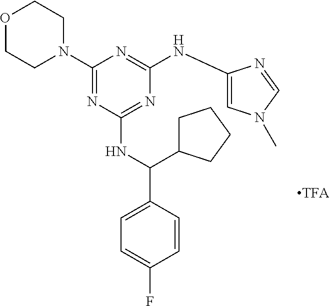

6-Chloro-N-[cyclopentyl(4-fluorophenyl)methyl]-N′-(1-methyl-1H-imidazol-4-yl)-1,3,5-triazine-2,4-diamine (Intermediate 44, 402 mg, 1.00 mmol) and morpholine (2 mL, 1.00 mmol), were reacted using a procedure similar to the one described for the synthesis of Example 1, providing the title product (130 mg) after purification using a Gilson® column (5%→85% MeCN/0.1% TFA in H₂O).

¹H NMR (300 MHz, MeOD) δ ppm 7.38 (m, 2H), 7.36 (br.s, 1H), 7.07 (m, 2H), 4.76 (d., 1H), 3.56-3.90 (m, 11H), 2.36 (m, 1H), 1.02-1.98 (m, 8H).

LCMS: 453 [M+H]⁺.

### Example 28

### 4-[(1S)-1-({4-[(1-methyl-1H-imidazol-4-yl)amino]-6-morpholin-4-yl-1,3,5-triazin-2-yl}amino)ethyl]benzonitrile, Trifluoroacetic Acid Salt

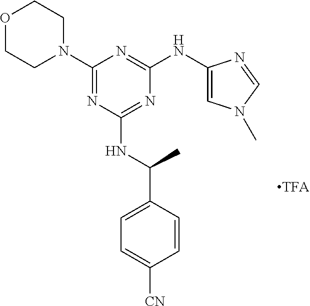

4-[(1S)-1-({4-Chloro-6-[(1-methyl-1H-imidazol-4-yl)amino]-1,3,5-triazin-2-yl}amino)ethyl]benzonitrile (Intermediate 45, 90 mg, 0.25 mmol) and morpholine (4 mL, 45.91 mmol) were reacted using a procedure similar to the one described for the synthesis of Example 1, providing the title product (111.7 mg) after purification using a Gilson® column (5%→85% MeCN/0.1% TFA in H₂O).

¹H NMR (300 MHz, MeOD) δ ppm 8.41 (brs. 1H), 7.71 (d., 2H), 7.59 (d, 2H), 7.26 (brs, 1H), 5.18 (q., 1H), 3.90 (s, 3H), 3.56-3.78 (m, 8H), 1.57 (d, 3H).

LCMS: 406 [M+H]⁺.

### Example 29

### N-[(1S)-1-(4-Chlorophenyl)ethyl]-N′-(1-methyl-1H-imidazol-4-yl)-6-morpholin-4-yl-1,3,5-triazine-2,4-diamine, Trifluoroacetic Acid Salt

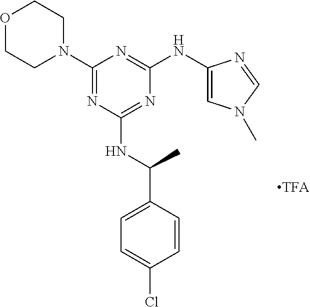

6-Chloro-N-[(1S)-1-(4-chlorophenyl)ethyl]-N′-(1-methyl-1H-imidazol-4-yl)-1,3,5-triazine-2,4-diamine (Intermediate 46, 743 mg, 2.04 mmol) and morpholine (5 mL, 57.39 mmol) were reacted using a procedure similar to the one described for the synthesis of Example 1, providing the title product (235.5 mg) after purification using a Gilson® column (5%→85% MeCN/0.1% TFA in H₂O).

¹H NMR (300 MHz, MeOD) δ ppm 8.39 (brs. 1H), 7.20-7.42 (m, 5H), 5.14 (q., 1H), 3.90 (s, 3H), 3.56-3.79 (m, 8H), 1.58 (d, 3H).

LCMS: 416 [M+H]⁺.

### Example 30

### N-[(1S)-1-(4-fluorophenyl)ethyl]-N′-(1-methyl-1H-imidazol-4-yl)-6-morpholin-4-yl-1,3,5-triazine-2,4-diamine, Trifluoroacetic Acid Salt

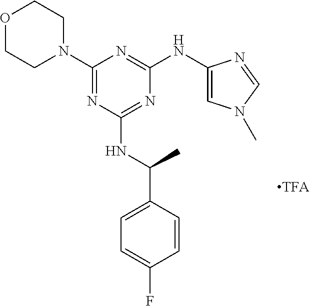

6-Chloro-N-[(1S)-1-(4-fluorophenyl)ethyl]-N′-(1-methyl-1H-imidazol-4-yl)-1,3,5-triazine-2,4-diamine (Intermediate 47, 709 mg, 2.04 mmol) and morpholine (5 mL, 57.39 mmol), were reacted using a procedure similar to the one described for the synthesis of Example 1, providing the title product (163.3 mg) after purification using a Gilson® column (5%→85% MeCN/0.1% TFA in H₂O).

¹H NMR (300 MHz, MeOD) δ ppm 8.39 (brs., 1H), 7.41 (t, 2H), 7.08 (t, 2H), 5.16 (q., 1H), 3.56-3.87 (m, 11H), 1.56 (d, 3H).

LCMS: 399 [M+H]⁺.

### Example 31

### N-[(1S)-1-(3,5-difluoropyridin-2-yl)ethyl]-N′-(1-ethyl-1H-imidazol-4-yl)-6-morpholin-4-yl-1,3,5-triazine-2,4-diamine hydrochloride

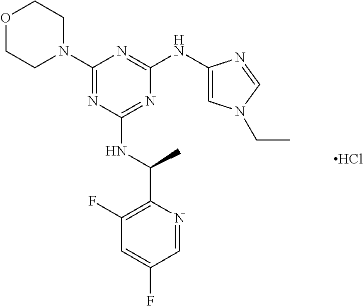

6-Chloro-N-[(1S)-1-(3,5-difluoropyridin-2-yl)ethyl]-N′-(1-ethyl-1H-imidazol-4-yl)-1,3,5-triazine-2,4-diamine (Intermediate 50, 0.42 mmol) and morpholine (2 mL, 22.96 mmol), were reacted using a procedure similar to the one described for the synthesis of Example 1, providing the product after purification using a Gilson® column (5%→60% MeCN/0.1% TFA in H₂O) and subsequent treatment of the evaporated fractions with 4N HCl in dioxane. Evaporation of the volatiles under reduced pressure afforded the title product. (139.4 mg).

¹H NMR (300 MHz, MeOD) δ ppm 8.87 (brs., 1H), 8.39 (d, 1H), 7.64 (ddd, 1H), 7.50 (brs, 1H), 5.54 (q., 1H), 4.26 (q, 2H), 3.64-3.91 (m, 8H), 1.55-1.59 (m, 6H).

LCMS: 432 [M+H]⁺.

### Example 32

### N-(1-Cyclopropyl-1H-imidazol-4-yl)-N′-[(1S)-1-(5-fluoropyrimidin-2-yl)ethyl]-6-morpholin-4-yl-1,3,5-triazine-2,4-diamine

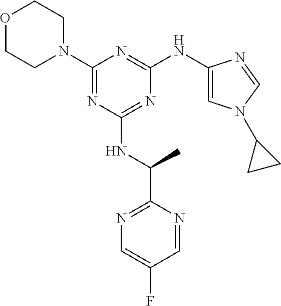

6-Chloro-N-(1-cyclopropyl-1H-imidazol-4-yl)-N′-[(1S)-1-(5-fluoropyrimidin-2-yl)ethyl]-1,3,5-triazine-2,4-diamine (Intermediate 53, 396 mg, 1.05 mmol) and morpholine (5 mL, 57.39 mmol) were reacted using a procedure similar to the one described for the synthesis of Example 1, providing the product (55 mg) after purification by column chromatography (ISCO, 0→100% ethyl acetate in hexanes).

¹H NMR (300 MHz, MeOD) δ ppm 9.00 (s. 1H), 8.77 (d, 2H), 7.64 (d, 1H), 5.34 (q., 1 H), 3.60-3.93 (m, 9H), 1.65 (d, 3H), 1.27 (d, 4H).

LCMS: 427 [M+H]⁺.

### Example 33

### N-[(1S)-1-(5-Fluoropyrimidin-2-yl)ethyl]-6-morpholin-4-yl-N′-{1-[2-(3 thienyl)ethyl]-1H-imidazol-4-yl}-1,3,5-triazine-2,4-diamine

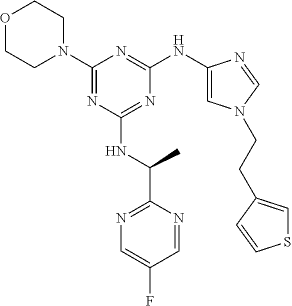

6-Chloro-N-[(1S)-1-(5-fluoropyrimidin-2-yl)ethyl]-N′-[1-(2-thien-3-ylethyl)-1H-imidazol-4-yl]-1,3,5-triazine-2,4-diamine (Intermediate 58, 0.130 g, 0.29 μmol) and morpholine (4 ml, 45.91 mmol) were reacted using a procedure similar to the one described for the synthesis of Example 1, providing the product (41.3 mg) after purification by column chromatography (ISCO, 0→100% ethyl acetate in hexanes).

¹H NMR (300 MHz, MeOD) δ ppm 8.70 (s, 2H), 7.40 (m, 1H), 7.20 (brs, 1H), 7.05 (brs, 1H), 6.92 (d, 1H), 5.25 (q, 1H), 4.23 (t, 2H), 3.56-3.76 (m, 8H), 3.16 (m, 2H), 1.58 (d, 3H).

LCMS: 497 [M+H]⁺.

### Example 34

### N-[(1S)-1-(5-fluoropyrimidin-2-yl)ethyl]-6-morpholin-4-yl-N′-[1-(2,2,2-trifluoroethyl)-1H-imidazol-4-yl]-1,3,5-triazine-2,4-diamine

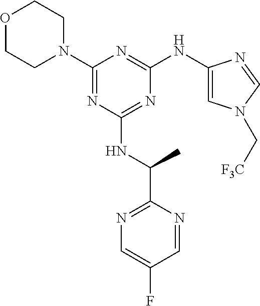

6-Chloro-N-[(1S)-1-(5-fluoropyrimidin-2-yl)ethyl]-N′-[1-(2,2,2-trifluoroethyl)-1H-imidazol-4-yl]-1,3,5-triazine-2,4-diamine (Intermediate 62, 668 mg, 1.6 mmol) and morpholine (4 mL, 45.91 mmol), were reacted using a procedure similar to the one described for the synthesis of Example 1, providing the product (220.8 mg) after purification by column chromatography (ISCO, 0→100% ethyl acetate in hexanes).

¹H NMR (300 MHz, MeOD) δ ppm 8.71 (s, 2H), 7.53 (s, 1H), 7.37 (brs, 1H), 5.26 (q, 1 H), 4.85 (m, 2H), 3.56-3.76 (m, 8H), 1.56 (d, 3H).

LCMS: 469 [M+H]⁺.

### Example 35

### N-(1-Ethyl-1H-imidazol-4-yl)-N′-[(1S)-1-(5-fluoropyrimidin-2-yl)ethyl]-6-morpholin-4-yl-1,3,5-triazine-2,4-diamine

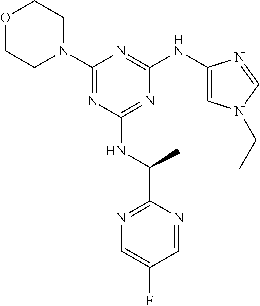

6-Chloro-N-(1-ethyl-1H-imidazol-4-yl)-N′-[(1S)-1-(5-fluoropyrimidin-2-yl)ethyl]-1,3,5-triazine-2,4-diamine (Intermediate 63, 702 mg, 1.93 mmol) and morpholine (5 mL, 57.39 mmol) were reacted using a procedure similar to the one described for the synthesis of Example 1, providing the product (344.2 mg) after purification by column chromatography (ISCO, 0→100% ethyl acetate in hexanes).

¹H NMR (300 MHz, MeOD) δ ppm 8.71 (s, 2H), 7.41 (s, 1H), 7.22 (brs, 1H), 5.29 (q, 1 H), 4.04 (q, 2H), 3.53-3.81 (m, 8H), 1.56 (d, 3H), 1.47 (t, 3H).

LCMS: 415 [M+H]⁺.

1. A compound of Formula (I) 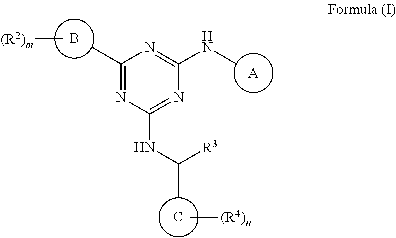
or a pharmaceutically acceptable salt thereof, wherein:
Ring A is selected from: 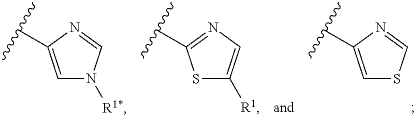
Ring B is 4 to 8-membered saturated heterocyclyl;
Ring C is selected from phenyl and 6-membered heteroaryl;
R¹ is selected from H, halo, —CN, C₁₋₆alkyl, C₂₋₆alkenyl, C₂₋₆alkynyl, carbocyclyl, heterocyclyl, —OR^{1a}, —SR^{1a}, —N(R^{1a})₂, —N(R^{1a})C(O)R^{1b}, —N(R^{1a})N(R^{1a})₂, —NO₂, —N(R^{1a})OR^{1a}, —ON(R^{1a})₂, —C(O)H, —C(O)R^{1b}, —C(O)₂R^{1a}, —C(O)N(R^{1a})₂, —C(O)N(R^{1a})(OR^{1a}), —OC(O)N(R^{1a})₂, —N(R^{1a})C(O)₂R^{1a}, —N(R^{1a})C(O)N(R^{1a})₂, —OC(O)R^{1b}, —S(O)R^{1b}, —S(O)₂R^{1b}, —S(O)₂N(R^{1a})₂, —N(R^{1a})S(O)₂R^{1b}, —C(R^{1a})═N(R^{1a}), and —C(R¹a)═N(OR¹a), wherein said C₁₋₆alkyl, C₂₋₆alkenyl, C₂₋₆alkynyl, carbocyclyl, and heterocyclyl are optionally substituted on carbon with one or more R¹⁰, and wherein any —NH— moiety of said heterocyclyl is optionally substituted with R¹⁰*;
R^{1a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
R^{1b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R¹⁰, and wherein any —NH— moiety of said heterocyclyl is optionally substituted with R¹⁰*;
R^{1c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
R¹* is selected from H, —CNC₁₋₆alkyl, carbocyclyl, heterocyclyl, —OR^{1a}, —C(O)H, —C(O)R^{1b}, —C(O)₂R^{1c}, —C(O)N(R^{1a})₂, —S(O)R^{1b}, —S(O)₂R^{1b}, —S(O)₂N(R^{1a})₂, —C(R^{10a})═N(R^{1a}), and —C(R^{1a})═N(OR^{1a}), wherein said C₁₋₆alkyl, carbocyclyl, and heterocyclyl are optionally substituted on carbon with one or more R¹⁰, and wherein any —NH— moiety of said heterocyclyl is optionally substituted with R¹⁰*;
R² in each occurrence is independently selected from halo, —CN, C₁₋₆alkyl, C₂₋₆alkenyl, C₂₋₆alkynyl, carbocyclyl, heterocyclyl, —OR^{2a}, —SR^{2a}, —N(R^{2a})₂, —N(R^{2a})C(O)R^{2b}, —N(R^{2a})N(R^{2a})₂, —NO₂, —N(R^{2a})OR^{2a}, —ON(R^{2a})₂, —C(O)H, —C(O)R^{2b}, —C(O)₂R^{2a}, —C(O)N(R^{2a})₂, —C(O)N(R^{2a})(OR^{2a})—OC(O)N(R^{2a})₂, —N(R^{2a})C(O)₂R^{2a}, —N(R^{2a})C(O)N(R^{2a})₂, —OC(O)R^{2b}, —S(O)R^{2b}, —S(O)₂R^{2b}, —S(O)₂N(R^{2a})₂, —N(R^{2a})S(O)₂R^{2b}, —C(R^{2a})═N(R^{2a}), and —C(R^{2a})═N(OR^{2a}), wherein said C₁₋₆alkyl, C₂₋₆alkenyl, C₂₋₆alkynyl, carbocyclyl, and heterocyclyl are optionally substituted on carbon with one or more R²⁰, and wherein any —NH— moiety of said heterocyclyl is optionally substituted with R²⁰*;
R^{2a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R²⁰, and wherein any —NH— moiety of said heterocyclyl is optionally substituted with R²⁰*;
R^{2b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R²⁰, and wherein any —NH— moiety of said heterocyclyl is optionally substituted with R²⁰*;
R³ is selected from H, halo, —CN, C₁₋₆alkyl, C₂₋₆alkenyl, C₂₋₆alkynyl, carbocyclyl, heterocyclyl, —OR^{3a}, —SR^{3a}, —N(R^{3a})₂, —N(R^{3a})C(O)R^{3b}, —N(R^{3a})N(R^{3a})₂, —NO₂, —N(R^{3a})—OR^{3a}, —O—N(R^{3a})₂, —C(O)H, —C(O)R^{3b}, —C(O)₂R^{3a}, —C(O)N(R^{3a})₂, —C(O)N(R^{3a})(OR^{3a}), —OC(O)N(R^{3a})₂, —N(R^{3a})C(O)₂R³, —N(R^{3a})C(O)N(R^{3a})₂, —OC(O)R^{3b}, —S(O)R^{3b}, —S(O)₂R^{3b}, —S(O)₂N(R^{3a})₂, —N(R^{3a})S(O)₂R^{3b}, —C(R^{3a})═N(R^{3a}), and —C(R^{3a})═N(OR^{3a}), wherein said C₁₋₆alkyl, C₂₋₆alkenyl, C₂₋₆alkynyl, carbocyclyl, and heterocyclyl are optionally substituted on carbon with one or more R³⁰, and wherein any —NH— moiety of said heterocyclyl is optionally substituted with R³⁰*;
R^{3a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R³⁰, and wherein any —NH— moiety of said heterocyclyl is optionally substituted with R³⁰*;
R^{3b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R³⁰, and wherein any —NH— moiety of said heterocyclyl is optionally substituted with R³⁰*;
R⁴ in each occurrence is independently selected from halo, —CN, C₁₋₆alkyl, C₂₋₆alkenyl, C₂₋₆alkynyl, carbocyclyl, heterocyclyl, —OR^{4a}, —SR^{4a}, —N(R^{4a})₂, —N(R^{4a})C(O)R^{4b}, —N(R^{4a})N(R^{4a})₂, —NO₂, —N(R^{4a})—OR^{4a}, —O—N(R^{4a})₂, —C(O)H, —C(O)R^{4b}, —C(O)₂R^{4a}, —C(O)N(R^{4a})₂, —C(O)N(R^{4a})(OR^{4a})—OC(O)N(R^{4a})₂, —N(R^{4a})C(O)₂R^{4a}, —N(R^{4a})C(O)N(R^{4a})₂, —OC(O)R^{4b}, —S(O)R^{4b}, —S(O)₂R^{4b}, —S(O)₂N(R^{4a})₂, —N(R^{4a})S(O)₂R^{4b}, —C(R^{4a})═N(R^{4a}), and —C(R^{4a})═N(OR^{4a}), wherein said C₁₋₆alkyl, C₂₋₆alkenyl, C₂₋₆alkynyl, carbocyclyl, and heterocyclyl are optionally substituted on carbon with one or more R⁴⁰, and wherein any —NH— moiety of said heterocyclyl is optionally substituted with R⁴⁰*;
R^{4a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁴⁰, and wherein any —NH— moiety of said heterocyclyl is optionally substituted with R⁴⁰*;
R^{4b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁴⁰, and wherein any —NH— moiety of said heterocyclyl is optionally substituted with R⁴⁰*;
R¹⁰ in each occurrence is independently selected from halo, —CN, C₁₋₆alkyl, C₂₋₆alkenyl, C₂₋₆alkynyl, carbocyclyl, heterocyclyl, —OR^{10a}, —SR^{10a}, —N(R^{10a})₂, —N(R^{10a})C(O)R^{10b}, —N(R^{10a})N(R^{10a})₂, —NO₂, —N(R^{10a})—OR^{10a}, —O—N(R^{10a})₂, —C(O)H, —C(O)R^{10b}, —C(O)₂R^{10a}, —C(O)N(R^{10a})₂, —C(O)N(R^{10a})(OR^{10a}), —OC(O)N(R^{10a})₂, —N(R^{10a})C(O)₂R^{10a}, —N(R^{10a})C(O)N(R^{10a})₂, —OC(O)R^{10b}, —S(O)R^{10b}, —S(O)₂R^{10b}, —S(O)₂N(R^{10a})₂, —N(R^{10a})S(O)₂R^{10b}, —C(R^{10a})═N(R^{10a}), and —C(R^{10a})═N(OR^{10a}), wherein said C₁₋₆alkyl, C₂₋₆alkenyl, C₂₋₆alkynyl, carbocyclyl, and heterocyclyl in each occurrence are optionally and independently substituted on carbon with one or more R^{a}, and wherein any —NH— moiety of said heterocyclyl is optionally substituted with R^{a}*;
R¹⁰* in each occurrence is independently selected from C₁₋₆alkyl, carbocyclyl, heterocyclyl, —C(O)H, —C(O)R^{10b}, —C(O)₂R^{10c}, —C(O)N(R^{10a})₂, —S(O)R^{10b}, —S(O)₂R^{10b}, —S(O)₂N(R^{10a})₂, —C(R^{10a})═N(R^{10a}), and —C(R^{10a})═N(OR^{10a}), wherein said C₁₋₆alkyl, carbocyclyl, and heterocyclyl in each occurrence are optionally and independently substituted on carbon with one or more R^{a}, and wherein any —NH— moiety of said heterocyclyl is optionally substituted with R^{a}*;
R^{10a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{a}, and wherein any —NH— moiety of said heterocyclyl is optionally substituted with R^{a}*;
R^{10b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{a}, and wherein any —NH— moiety of said heterocyclyl is optionally substituted with R^{a}*;
R^{10c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{a}, and wherein any —NH— moiety of said heterocyclyl is optionally substituted with R^{a}*;
R²⁰ in each occurrence is independently selected from halo, —CN, C₁₋₆alkyl, C₂₋₆alkenyl, C₂₋₆alkynyl, carbocyclyl, heterocyclyl, —OR^{20a}, —SR^{20a}, —N(R^{20a})₂, N(R^{20a})C(O)R^{20b}, —N(R^{20a})N(R^{20a})₂, —NO₂, —N(R^{20a})—OR^{20a}, —O—N(R^{20a})₂, —C(O)H, —C(O)R^{20b}, —C(O)₂R^{20a}, —C(O)N(R^{20a})₂, —C(O)N(R^{20a})(OR^{20a}), —OC(O)N(R^{20a})₂, —N(R^{20a})C(O)₂R^{20a}, —N(R^{20a})C(O)N(R^{20a})₂, —OC(O)R^{20b}, —S(O)R^{20b}, —S(O)₂R^{20b}, —S(O)₂N(R^{20a})₂, —N(R^{20a})S(O)₂R^{20b}, —C(R^{20a})═N(R^{20a}), and —C(R^{20a})═N(OR^{20a}), wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein any —NH— moiety of said heterocyclyl is optionally substituted with R^{b}*;
R²⁰* in each occurrence is independently selected from C₁₋₆alkyl, carbocyclyl, heterocyclyl, —C(O)H, —C(O)R^{20b}, —C(O)₂R^{20c}, —C(O)N(R^{20a})₂, —S(O)R^{20b}, —S(O)₂R^{20b}, —S(O)₂N(R^{20a})₂, —C(R^{20a})═N(R^{20a}), and —C(R^{20a})═N(OR^{20a}), wherein said C₁₋₆alkyl, carbocyclyl, and heterocyclyl in each occurrence are optionally and independently substituted on carbon with one or more R^{b}, and wherein any —NH— moiety of said heterocyclyl is optionally substituted with R^{b}*;
R^{20a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{b}, and wherein any —NH— moiety of said heterocyclyl is optionally substituted with R^{b}*;
R^{20b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein any —NH— moiety of said heterocyclyl is optionally substituted with R^{b}*;
R^{20c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{b}, and wherein any —NH— moiety of said heterocyclyl is optionally substituted with R^{b}*;
R³⁰ in each occurrence is independently selected from halo, —CN, C₁₋₆alkyl, C₂₋₆alkenyl, C₂₋₆alkynyl, carbocyclyl, heterocyclyl, —OR^{30a}, —SR^{30a}, —N(R^{30a})₂, —N(R^{30a})C(O)R^{30b}, —N(R^{30a})N(R^{30a})₂, —NO₂, —N(R^{30a})—OR^{30a}, —O—N(R^{30a})₂, —C(O)H, —C(O)R^{30b}, —C(O)₂R^{30a}, —C(O)N(R^{30a})₂, —C(O)N(R^{30a})(OR^{30a}), —OC(O)N(R^{30a})₂, —N(R^{30a})C(O)₂R^{30a}, —N(R^{30a})C(O)N(R^{30a})₂, —OC(O)R^{30b}, —S(O)R^{30b}, —S(O)₂R^{30b}, —S(O)₂N(R^{30a})₂, —N(R^{30a})S(O)₂R^{30b}, —C(R^{30a})═N(R^{30a}), and —C(R^{30a})═N(OR^{30a}), wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein any —NH— moiety of said heterocyclyl is optionally substituted with R^{c}*;
R³⁰* in each occurrence is independently selected from C₁₋₆alkyl, carbocyclyl, heterocyclyl, —C(O)H, —C(O)R^{30b}, —C(O)₂R^{30c}, —C(O)N(R^{30a})₂, —S(O)R^{30b}, —S(O)₂R^{30b}, —S(O)₂N(R^{30a})₂, —C(R^{30a})═N(R^{30a}), and —C(R^{30a})═N(OR^{30a}), wherein said C₁₋₆alkyl, carbocyclyl, and heterocyclyl in each occurrence are optionally and independently substituted on carbon with one or more R^{c}, and wherein any —NH— moiety of said heterocyclyl is optionally substituted with R^{c}*;
R^{30a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{c}, and wherein any —NH— moiety of said heterocyclyl is optionally substituted with R^{c}*;
R^{30b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein any —NH— moiety of said heterocyclyl is optionally substituted with R^{c}*;
R^{30c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{c}, and wherein any —NH— moiety of said heterocyclyl is optionally substituted with R^{c}*;
R⁴⁰ in each occurrence is independently selected from halo, —CN, C₁₋₆alkyl, C₂₋₆alkenyl, C₂₋₆alkynyl, carbocyclyl, heterocyclyl, —OR^{40a}, —SR^{40a}, —N(R^{40a})₂, —N(R^{40a})C(O)R^{40b}, —N(R^{40a})N(R^{40a})₂, —NO₂, —N(R^{40a})—OR^{40a}, —O—N(R^{40a})₂, —C(O)H, —C(O)R^{40b}, —C(O)₂R^{40a}, —C(O)N(R^{40a})₂, —C(O)N(R^{40a})(OR^{40a}), —OC(O)N(R^{40a})₂, —N(R^{40a})C(O)₂R^{40a}, —N(R^{40a})C(O)N(R^{40a})₂, —OC(O)R^{40b}, —S(O)R^{40b}, —S(O)₂R^{40b}, —S(O)₂N(R^{40a})₂, —N(R^{40a})S(O)₂R^{40b}, —C(R^{40a})═N(R^{40a}), and —C(R^{40a})═N(OR^{40a}), wherein said C₁₋₆alkyl, C₂₋₆alkenyl, C₂₋₆alkynyl, carbocyclyl, and heterocyclyl in each occurrence are optionally and independently substituted on carbon with one or more R^{d}, and wherein any —NH— moiety of said heterocyclyl is optionally substituted with R^{d}*;
R⁴⁰* in each occurrence is independently selected from C₁₋₆alkyl, carbocyclyl, heterocyclyl, —C(O)H, —C(O)R^{40b}, —C(O)₂R^{40c}, —C(O)N(R^{40a})₂, —S(O)R^{40b}, —S(O)₂R^{40b}, —S(O)₂N(R^{40a})₂, —C(R^{40a})═N(R^{40a}), and —C(R^{40a})═N(OR^{40a}), wherein said C₁₋₆alkyl, carbocyclyl, and heterocyclyl in each occurrence are optionally and independently substituted on carbon with one or more R^{d}, and wherein any —NH— moiety of said heterocyclyl is optionally substituted with R^{d}*;
R^{4a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{d}, and wherein any —NH— moiety of said heterocyclyl is optionally substituted with R^{d}*;
R^{40b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{d}, and wherein any —NH— moiety of said heterocyclyl is optionally substituted with R^{d}*;
R^{40c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{d}, and wherein any —NH— moiety of said heterocyclyl is optionally substituted with R^{d}*;
R^{a}, R^{b}, R^{c}, and R^{d} in each occurrence are independently selected from halo, —CN, C₁₋₆alkyl, C₂₋₆alkenyl, C₂₋₆alkynyl, carbocyclyl, heterocyclyl, —OR^{m}, —SR^{m}, —N(R^{m})₂₅—N(R^{m})C(O)Rⁿ, —N(R^{m})N(R^{m})₂, —NO₂, —N(R^{m})—OR^{m}, —O—N(R^{m})₂, —C(O)H, —C(O)Rⁿ, —C(O)₂R^{m}, —C(O)N(R^{m})₂, —C(O)N(R^{m})(OR^{m}), —OC(O)N(R^{m})₂, —N(R^{m})C(O)₂R^{m}, —N(R^{m})C(O)N(R^{m})₂, —OC(O)Rⁿ, —S(O)Rⁿ, —S(O)₂Rⁿ, —S(O)₂N(R^{m})₂, —N(R^{m})S(O)₂Rⁿ, —C(R^{m})═N(R^{m}), and —C(R^{m})═N(OR^{m});
R^{a}*, R^{b}*, R^{c}*, and R^{d}*in each occurrence are independently selected from C₁₋₆alkyl, carbocyclyl, heterocyclyl, —C(O)H, —C(O)Rⁿ, —C(O)₂R^{o}, —C(O)N(R^{m})₂, —S(O)Rⁿ, —S(O)₂Rⁿ, —S(O)₂N(R^{m})₂, —C(R^{m})═N(R^{m}), and —C(R^{m})═N(OR^{m});
R^{m} in each occurrence is independently selected from H, C₁₋₆alkyl, carbocyclyl, and heterocyclyl;
Rⁿ in each occurrence is independently selected from C₁₋₆alkyl, C₂₋₆alkenyl, C₂₋₆alkynyl, carbocyclyl, and heterocyclyl;
R^{o} in each occurrence is independently selected from C₁₋₆alkyl, carbocyclyl, and heterocyclyl; and
m is selected from 0, 1, 2, 3, 4, 5, and 6; and
n is selected from 1, 2, 3, and 4.

2. A compound of Formula (I), or a pharmaceutically acceptable salt thereof, as claimed in claim 1, wherein
Ring A is selected from 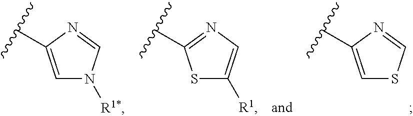
R¹ is selected from —CN and C₁₋₆alkyl;
R¹* is selected from 3- to 6-membered carbocyclyl and C₁₋₆alkyl, wherein said C₁₋₆alkyl is optionally substituted on carbon with one or more R¹⁰;
R¹⁰ in each occurrence is independently selected from halo, —CN, 3- to 6-membered carbocyclyl, 4- to 6-membered heterocyclyl, and —OR^{10a};
R^{10a} is C₁₋₆alkyl.

3. A compound of Formula (I), or a pharmaceutically acceptable salt thereof, as claimed in claim 1, wherein
Ring B is 4 to 6-membered saturated heterocyclyl;
R² in each occurrence is independently selected from halo, C₁₋₆alkyl, and —OR^{2a}, wherein said C₁₋₆alkyl in each occurrence is optionally and independently substituted with one or more R²⁰;
R^{2a} is C₁₋₆alkyl;
R²⁰ is —OH; and
m is selected from 0, 1, 2.

4. A compound of Formula (I), or a pharmaceutically acceptable salt thereof, as claimed in claim 1, wherein
Ring C is selected from phenyl and 6-membered heteroaryl;
R⁴ in each occurrence is independently selected from halo and —CN; and
n is selected from 1 and 2.

5. A compound of Formula (I), or a pharmaceutically acceptable salt thereof, as claimed in claim 1, wherein
R³ is selected from C₁₋₆alkyl, 3 to 6-membered carbocyclyl, and 4 to 6-membered heterocyclyl, wherein said C₁₋₆alkyl is optionally substituted with one or more R³⁰, and wherein any —NH— moiety of said 4 to 6-membered heterocyclyl is optionally substituted with R³⁰*;
R³⁰ in each occurrence is independently selected from —OR^{30a};
R³⁰* is C₁₋₆alkyl; and
R^{30a} is C₁₋₆alkyl.

6. A compound of Formula (I): 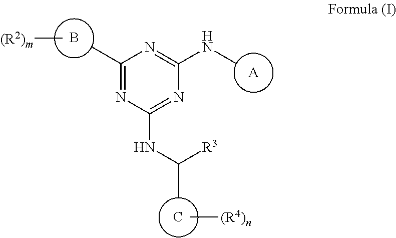
or a pharmaceutically acceptable salt thereof, wherein:
Ring A is selected from 1-(cyanomethyl)-1H-imidazol-4-yl, 5-cyano-1,3-thiazol-2-yl, 1-cyclopropyl-1H-imidazol-4-yl, 1-ethyl-1H-imidazol-4-yl, 1-isopropyl-1H-imidazol-4-yl, 1H-imidazol-4-yl, 1-(methoxymethyl)-1H-imidazol-4-yl, 1-methyl-1H-imidazol-4-yl, 5-methyl-1,3-thiazol-2-yl, 1-(2-phenylethyl)-1H-imidazol-4-yl, 1,3-thiazol-4-yl, 1-[2-(3-thienyl)ethyl]-1H-imidazol-4-yl, and 1-(2,2,2-trifluoroethyl)-1H-imidazol-4-yl;
Ring B, R², and m together form a group selected from 4,4-difluoropiperidin-1-yl, 2,2-dimethylmorpholin-4-yl, 2,6-dimethylmorpholin-4-yl, 2-methylmorpholin-4-yl, 3-fluoroazetidin-1-yl, 4-fluoropiperidin-1-yl, 3-(hydroxymethyl)morpholin-4-yl, 3-methoxyazetidin-1-yl, and morpholin-4-yl;
Ring C, R⁴, and n together form a group selected from 4-chlorophenyl, 4-cyanophenyl, 3,5-difluoropyridin-2-yl, 4-fluorophenyl, and 5-fluoropyrimidin-2-yl; and
R³ is selected from cyclopentyl, methoxymethyl, methyl, and 1-methyl-1H-imidazol-4-yl.

7. (canceled)

8. (canceled)

9. A method for treating cancer in a warm-blooded animal such as man, said method comprising administering to said animal an effective amount of a compound of Formula (I), or a pharmaceutically acceptable salt thereof, as claimed in claim 1.

10. (canceled)

11. A pharmaceutical composition comprising a compound of Formula (I), or a pharmaceutically acceptable salt thereof, as claimed in claim 1, and at least one pharmaceutically acceptable carrier, diluent, or excipient.

12. A process for preparing a compound of Formula (I), or a pharmaceutically acceptable salt thereof, as claimed in claim 1, wherein said process is selected from:
Process A—reacting a compound of Formula (A): 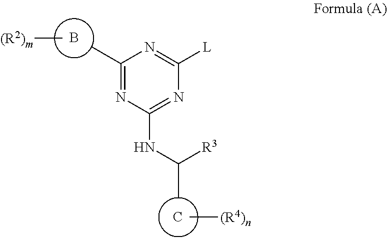
with a compound of Formula (B): 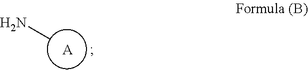
Process B—reacting a compound of Formula (C) 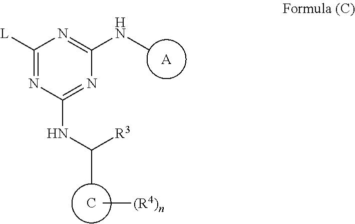
with a compound of Formula (D) 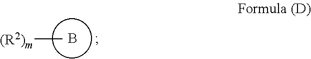
Process C—reacting a compound of Formula (E) 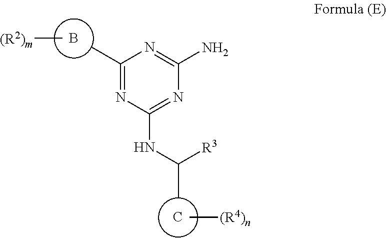
with a compound of Formula (F) 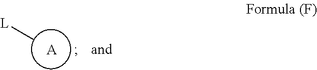
Process D—reacting a compound of Formula (G) 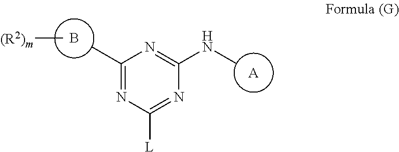
with a compound of Formula (H) 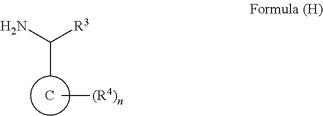
and thereafter if appropriate:
i) converting a compound of Formula (I) into another compound of Formula (I);
ii) removing any protecting groups; and/or
iii) forming a pharmaceutically acceptable salt,
wherein L in each occurrence may be the same or different, and is a leaving group.
